(19) United States
(12) Patent Application Publication
(10) Pub. No.: US 2025/0270619 A1
(43) Pub. Date: Aug. 28, 2025

(54) COMPOSITIONS, KITS AND METHODS FOR DIRECT AMPLIFICATION FROM CRUDE BIOLOGICAL SAMPLES

(71) Applicant: LIFE TECHNOLOGIES CORPORATION, Carlsbad, CA (US)

(72) Inventors: Mark SHANNON, San Francisco, CA (US); Noah ELDER, Lafayette, CA (US); Kalpith RAMAMOORTHI, San Jose, CA (US); Jason LA, San Bruno, CA (US); Daniel BLANCHARD, South San Francisco, CA (US)

(21) Appl. No.: 18/261,812

(22) PCT Filed: Jan. 18, 2022
(86) PCT No.: PCT/US2022/012755

§ 371 Date: Jul. 17, 2023

Related U.S. Application Data
(60) Provisional application No. 63/199,693, filed on Jan. 18, 2021.

(51) Int. Cl. C12Q 1/6806; C12Q 1/6851; C12Q 1/70

(52) U.S. Cl. CPC C12Q 1/6806; C12Q 1/6851; C12Q 1/701

(57) ABSTRACT

Disclosed are compositions, assays, methods, diagnostic methods, kits and diagnostic kits for the detection of target nucleic acids, including those from microbes and/or from infectious agents such as SARS-CoV-2 and other viruses. Embodiments described herein are designed to enable processing and analysis of the sample to detect target nucleic acids within the sample without requiring extraction and/or isolation of nucleic acid from the sample prior to subsequent processing steps. Samples analyzed can thus be “crude” biological samples that do not require pre-processing prior to placement in the workflow.

### CROSS-REFERENCE TO RELATED APPLICATIONS

This application claims priority to and the benefit of U.S. Provisional Patent Application No. 63/199,693 filed Jan. 18, 2021, the entirety of which is incorporated herein by this reference.

### SEQUENCE LISTING

This application includes a Sequence Listing submitted electronically in ASCII format. The ASCII copy of the Sequence Listing, created on Jan. 3, 2022, is named LT01570PCT-WN16100.47-SL.txt and is 269,200 bytes in size. The ASCII copy of the Sequence Listing is expressly incorporated herein by this reference.

### FIELD

The present teachings relate to compositions, methods, systems, and kits for amplification, detection, diagnosis, and/or differentiation of microbes and/or infectious agents (e.g., viruses) in a biological sample. In particular, the present disclosure is related to the detection of target nucleic acids sequences, such as from a virus, in a crude biological sample without requiring an initial step of extracting the nucleic acid from the sample.

### BACKGROUND

Infectious diseases are caused by pathogenic microbes or infectious agents (e.g., viruses). Early and accurate diagnosis of infectious disease is important for several reasons. For example, proper diagnosis can lead to earlier, more effective treatment which improves outcomes for the infected individual. On the other hand, individuals who are undiagnosed or misdiagnosed may unknowingly transmit diseases to others. Accurate diagnoses also help ensure proper treatments are applied, particularly with respect to certain disease categories with multiple pathogenic causes and similar symptom profiles, such as respiratory diseases.

One example of a problematic virus associated with infectious diseases are coronaviruses. Coronaviruses are a family of viruses having a single stranded positive sense RNA genome of about 30 kilobases in length. Human coronaviruses were first identified in the mid 1960's as being one of the many etiologic agents of the common cold. People around the world commonly get infected with human coronavirus strains 229E (an alpha coronavirus), NL63 (an alpha coronavirus), OC43 (a beta coronavirus), and HKU1 (a beta coronavirus). These infections present with mild clinical symptoms and are associated with an extremely low mortality rate.

Some coronaviruses infect non-human animals where they can evolve and undergo zoonosis, expanding their tropism to humans. Such crossover events have proven devastating in years past. For example, the Middle East Respiratory Syndrome (MERS) was caused by MERS-CoV, a beta coronavirus that crossed over from dromedary camels to humans. MERS-CoV was associated with a high mortality rate of approximately 35%, but its low transmissibility rate helped to limit its spread and potential for devastation. As another example, Severe Acute Respiratory Syndrome (SARS), which was caused by SARS-CoV, another beta coronavirus, was believed to have been transmitted from bats to civet cats who then transmitted the virus to humans. Although not as deadly as MERS-CoV, SARS-CoV was nevertheless associated with a moderately high mortality rate of approximately 9.6%. Likely due, at least in part, to the lifecycle of SARS-CoV within humans, the spread of this virus was limited mostly to Southeast Asian countries. Human infected with SARS-CoV often became symptomatic prior to shedding infectious virions, making quarantining a particularly useful tool for limiting exposure and spread of the infection.

More recently, a new variant beta coronavirus, SARS-CoV-2 (also known as 2019-nCoV), has emerged, potentially from a crossover event between pangolins or bats and humans in Wuhan, China. While the epidemiological data are incomplete, reports at the time of this writing indicate that over 293 million people worldwide are believed to have already been infected by SARS-CoV-2. However, unlike MERS-CoV and SARS-CoV before it, SARS-CoV-2 appears to be significantly less lethal on average. Due to its increased transmissibility, the seemingly small percentage of deaths associated with SARS-CoV-2 belies its worldwide impact, having caused an estimated 5.45 million deaths in the worldwide pandemic at the time of this writing, and currently continuing to grow. The raw number of humans impacted by SARS-CoV-2 dwarfs the total number of deaths caused by MERS-CoV and SARS-CoV combined-reportedly around 1,600.

Given the present and continuing emergence of new coronavirus strains, there is an urgent need to develop methods for the rapid detection and characterization of existing and novel coronavirus strains so that appropriate treatment and infection control measures can be properly instituted in a timely manner. Problematically, many of the SARS-CoV-2 detection assays require multiple steps and a lengthy workflow. Common problems include long processing times (and thus long wait times for patient results), the requirement for lots of hands-on time by practitioners or technicians (thereby using up personnel and infrastructure resources), and rigid sample treatment protocols that lack flexibility in treatment time, treatment temperature, or other processing parameters. These limitations raise the risk of mistakes, misdiagnoses, and/or the requirement for repeated tests. Each misidentified, misdiagnosed, or late/untimely diagnosed instance of SARS-CoV-2 infection further convolutes the epidemiological data and prevents the implementation of appropriate, informed solutions.

Moreover, safe transport and handling of pathogenic enveloped viruses such as betacoronaviruses necessitates processes that inactivates the virus in a sample prior to transport and subsequent processing. Methods of inactivating enveloped viruses include the application of heat, ionizing radiation, or non-ionizing radiation to a sample. Chemical treatments include exposure of a sample to a solution at low pH (<4) or high pH (>10), chaotropic salts such as guanidinium isothiocyanate, cross-linking agents such as formaldehyde or glutaraldehyde, alcohols such as ethanol or methanol, and exposure to detergents.

One approach that seeks to simplify detection workflows is “direct” detection of nucleic acids from microbes or infectious agents, such as a virus, in a crude, unpurified sample. While this approach can offer several benefits, it can be difficult for several reasons. First, the various methods used to inactivate the microbe or infections agents, such as in the case of enveloped viruses, can lead to damage or destruction of the nucleic acids in the sample. Second, chemicals and biological agents used to inactivate microbes and/or infectious agents such as viruses can interfere with or inhibit subsequent detection of the target nucleic acids, such as during PCR. Third, components of a crude, unpurified sample can interfere with or inhibit nucleic acid detection systems. It is thus very challenging to meet the requirements of providing proper inactivation of microbes and/or infectious agents such as virions within a crude sample, sufficient release of target nucleic acids, and adequate stabilization of released nucleic acids, all without overly interfering with subsequent detection processes.

Accordingly, there are a number of disadvantages with current methods, systems, compositions, and kits for detecting target nucleic acids in a crude biological sample that can be addressed.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope.

The disclosure will be described and explained with additional specificity and detail through the use of the accompanying drawings in which:

FIGS. 1A-1C illustrate C_{q} values of an RT-qPCR procedure screening multiple surfactants to test capability of disrupting SARS-Cov-2 virus particles, as determined by detection of released target nucleic acids;

FIG. 2A-2C illustrate C_{q} values of an RT-qPCR procedure to test the effect of surfactant concentration on virus disruption activity;

FIG. 3 illustrates C_{q} values of an RT-qPCR procedure to test the effect of surfactant treatment solution incubation time on virus disruption activity;

FIG. 4 illustrates C_{q} values of an RT-qPCR procedure to test the effect of including a protease in the surfactant treatment solution;

FIG. 5 illustrates results of a test determining SARS-CoV-2 virus titer reduction in Vero E6 cells using treatment solutions of the present disclosure;

FIG. 6 illustrates C_{q} values of an RT-qPCR procedure detecting SARS-CoV-2 RNA in raw saliva samples that contain virus particles without the need for a nucleic acid purification step;

FIG. 7 illustrates C_{q} values of an RT-qPCR procedure detecting SARS-CoV-2 RNA in raw saliva samples, comparing the use of proteinase K and pronase as protease components;

FIGS. 8A through 8H illustrate amplification plots of an RT-qPCR procedure detecting SARS-CoV-2 RNA in raw saliva samples that contain virus particles without the need for a nucleic acid purification step;

FIGS. 9A-9C illustrates the results of stability testing, showing that SARS-CoV-2 RNA remains stable in mixtures of saliva and a treatment solution containing surfactant and protease;

FIGS. 10A through 10D illustrate amplification plots of an RT-qPCR procedure detecting SARS-CoV-2 RNA from dry nasal and oropharyngeal swabs resuspended in treatment solution;

FIG. 11 compares C_{q} values of an RT-qPCR procedure using a treatment solution according to the present disclosure to procedures using conventional approaches, showing that the disclosed treatment solution and process provides results with greater uniformity and greater sensitivity as compared to the conventional approaches;

FIG. 12 illustrates results of testing three saliva samples confirmed as positive for SARS-CoV-2 using the disclosed treatment solution and method compared to a conventional method, showing that the disclosed treatment solution and method provide more effective results; and

FIGS. 13A and 13B illustrate amplification plots of an RT-qPCR procedure using a treatment solution according to the present disclosure and a single vessel protocol, showing effective results.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

All publications and patent applications cited herein, as well as the Appendices attached hereto, are incorporated by reference in their entirety for all purposes to the same extent as if each were specifically and individually indicated to be so incorporated by reference. Although the present invention has been described in some detail by way of illustration and example for purposes of clarity and understanding, it will be apparent that certain changes and modifications may be practiced within the scope of the spirit and substance of this disclosure and of the appended claims.

Any headings and subheadings used herein are for organizational purposes only and are not meant to be used to limit the scope of the description or the claims.

### Overview of Compositions, Systems, and Kits for Detection of Target Sequences

As discussed above, current detection assays used to identify target nucleic acids from microbes or viruses (such as SARS-CoV-2) include several limitations, including often requiring complex workflows, long processing times, multiple upfront sample collection and processing steps, and rigid sample treatment protocols that lack flexibility in treatment time, treatment temperature, or other processing parameters. These limitations raise the risk of mistakes, misdiagnoses, and/or the requirement for repeated tests. Moreover, even when performed accurately and within the rigid protocol requirements, the overall complexity of the associated workflows represent a significant cost to personnel and infrastructure resources.

The lack of a flexible, reliable assay for accurately identifying target microbes and/or viruses in a sample (e.g., a clinical sample obtained from nasopharyngeal swab, nasopharyngeal aspirate, bronchoalveolar lavage, buccal swab, saliva, or urine) and/or differentiating one target from another (e.g., one virus from other viruses) also prevents healthcare professionals from properly treating and advising patients. Given the present and continuing emergence of new microbes and infectious agents, including new coronavirus strains as well as other existing and emergent viruses such as influenza and respiratory syncytial virus (RSV), there is an urgent need to develop more flexible and less complex methods for the rapid detection and characterization of existing and novel targets so that appropriate treatment and infection control measures can be properly instituted in a timely manner.

Disclosed herein are compositions, kits, and methods for “directly” detecting target nucleic acid sequences. In some embodiments the target nucleic acid is from a microbe or infectious agent such as a virus, a bacterium, or a fungus (or multiples thereof and/or some combination thereof). In some embodiments the target nucleic acid is from a virus, particularly an enveloped virus such as a coronavirus (e.g., SARS-CoV-2). Although several of the specific examples relate to detection of nucleic acid sequences associated with SARS-CoV-2, the skilled person will understand that features taught and described in relation thereto are also applicable to implementations where target nucleic acids are associated with other targets, in addition to or alternative to SARS-CoV-2 targets. The other targets may include nucleic acids from bacteria, fungi, viruses, or samples with multiple types of target microbes and/or viruses. Examples of targets include, but are not limited to, Bordetella (PAN), Bordetella holmesii, Bordetella pertussis, Chlamydophila pneumoniae, Mycoplasma pneumoniae, Streptococcus pneumoniae, Coxiella burnetiid, Staphylococcus aureus, Klebsiella pneumoniae, Legionella pneumophila, Moraxella catarrhalis, Haemophilus influenzae, Pneumocystis jirovecii, adenovirus, coronavirus HKU1, coronavirus NL63, coronavirus 229E, coronavirus OC43, human metapneumovirus, rhinovirus, enterovirus, enterovirus D68, influenza A (Pan), influenza A/H1-2009, influenza A/H3, influenza B, parainfluenza 1, parainfluenza 2, parainfluenza 3, parainfluenza 4, RSA A, RSA B, bocavirus, Epstein-Barr virus (EBV), SARS-CoV, MERS-CoV, mumps, measles, cytomegalovirus, human herpesvirus 6 (HHV-6), varicella zoster virus (VZV), and parechovirus.

Embodiments described herein are designed to enable processing and analysis of the sample to detect targeted nucleic acids within the sample without requiring extraction and/or isolation of nucleic acid from the sample prior to subsequent processing steps. Samples analyzed can thus be “crude” biological samples that do not require pre-processing prior to placement in the workflow. Such “crude” samples may include, for example, a saliva sample, buccal sample, nasal sample, nasal pharyngeal sample, blood sample, urine sample, fecal sample, or semen sample.

The embodiments described herein beneficially reduce the overall number of processing steps, reduce the complexity of the workflow, allow faster time to results as compared to conventional protocols requiring nucleic acid extraction, reduce the amount of hands-on time required of personnel, require fewer consumables as compared to standard extraction-based protocols, and provide relative flexibility in sample treatment time, sample treatment temperature, and/or other processing parameters.

Other exemplary features include one or more of: minimal sample dilution requirements (and thus higher sample concentration inputs); flexible reaction size; compatibility across multiple types of samples; minimal or no loss of specificity as compared to standard extraction-based protocols; and compatibility with standard sample storage and shipping conditions. Treatment solutions used in the disclosed embodiments are beneficially formulated to inactivate target microbes and/or viruses (such as SARS-CoV-2) with minimal inhibition. The foregoing features ultimately lead to benefits such as lower demand on personnel and infrastructure resources, faster delivery of results to patients and practitioners, and reduced risk of mishandled and/or misdiagnosed samples.

### Treatment Solution Formulations

Treatment solutions (also referred to herein as “treatment buffers” or “treatment compositions”) are formulated for mixing with a crude biological sample to enable subsequent analysis of a target nucleic acid within (or suspected of being within) the sample. In one embodiment, the treatment buffer includes a surfactant, a protease component, a chelating agent, and a buffering salt. The treatment buffer may also optionally include a saccharide, preferably a disaccharide such as sucrose, trehalose, or combination thereof. When a saccharide is included, it is typically most effective when included at a concentration of about 200 mM to about 600 mM.

The surfactant may include a nonionic detergent, a cationic detergent, a zwitterionic detergent, an anionic detergent, or any combination thereof (though anionic detergents are typically less preferred due to their tendency to interfere with downstream PCR). Non-limiting examples of suitable nonionic detergents include nonyl phenoxypolyethoxylethanol (NP-40), secondary alcohol ethoxylates such as TERGITOL 15-S-9 or TERGITOL 15-S-40 (TERGITOL 15-S-9 being more preferred), Triton X-100 (i.e., 2-[4-(2,4,4-trimethylpentan-2-yl) phenoxy]ethanol), and TWEEN 20 (generically named polysorbate 20). Non-limiting examples of suitable cationic detergents include benzalkonium chloride (BZK) and didodecyldimethylammonium bromide (DDAB). Non-limiting examples of suitable zwitterionic detergents include lauryldimethylamine oxide (i.e., LDAO, DDAO), N-(Alkyl C₁₀-C₁₆)-N,N-dimethylglycine betaine (sold under the trade name EMPIGEN BB), n-Tetradecyl-N,N-dimethyl-3-ammonio-1-propanesulfonate (sold under the trade name ZWITTERGENT 3-14), CHAPS (i.e., 3-[(3-cholamidopropyl)dimethylammonio]-1-propanesulfonate), or CHAPSO (i.e., 3-[(3-cholamidopropyl)dimethylammonio]-2-hydroxy-1-propanesulfonate). More preferred zwitterionic detergents include LDAO, EMPIGEN BB, and ZWITTERGENT 3-14. Combinations of any of the foregoing surfactants may also be utilized. As shown in detail in the Examples below, surfactants which have proven to be particularly effective in subsequent detection of target nucleic acid (e.g., from SARS-CoV-2), include LDAO and BZK.

The surfactant may be included at a concentration of about 0.01% to about 0.10% w/v, or more preferably about 0.02% to about 0.08% w/v. Another way to determine an appropriate surfactant concentration is to include the surfactant at a concentration within 0.5× and 15× of the surfactant's critical micelle concentration (CMC). One of skill in the art will understand how to conduct a CMC test and/or consult appropriate literature to find such values for a selected surfactant.

The protease component may include a serine protease such as proteinase K. In some embodiments, the protease component includes a mixture of two or more proteases. The protease mixture may comprise a mixture of proteases isolated from a bacterial culture. One example of such a mixture is pronase, which is a mixture of proteases isolated from extracellular fluid of the actinobacteria Streptomyces griseus. As shown in detail in the Examples below, pronase has proven to be particularly effective in increasing the accessibility to viral nucleic acids in crude samples. Other proteases, including other proteases from other types of microbial cultures, may additionally or alternatively be utilized in the protease component. The protease component may be included at a concentration of about 20 U/ml to about 100 U/ml, or more preferably about 35 U/ml to about 85 U/ml, or even more preferably about 50 U/ml to about 70 U/ml.

The surfactant, protease component, or both function to inactivate virions (and/or other infectious agents or microorganisms) within the sample. The inactivation effects of the surfactant and protease component were surprisingly found to be enhanced when utilized in combination as compared to when each component was used independently, other conditions being equal (see, e.g., Example 5 below). The treatment solution also functions to disrupt viral envelopes, cell membranes, or proteins within the crude biological sample. The treatment solution beneficially provides increased access to the target nucleic acid when mixed with the crude biological sample as compared to a mixture of the crude biological sample omitting one or more components of the treatment solution (e.g., as compared to a mixture of the sample with water and/or buffer only).

The buffering salt may include any salt or salt mixture that provides sufficient buffering functionality. Suitable salts include sodium salts (e.g., sodium citrate) and/or chloride salts (e.g., Tris-HCl). The salt concentration is preferably less than about 50 mM, such as within a range with a lower endpoint of about 2 mM and an upper endpoint of about 40 mM, 30 mM, 20 mM, or 15 mM. The chelating agent may include ethylenediaminetetraacetic acid (EDTA) or a conjugate base or salt thereof, for example. The chelating agent may be included at a concentration of about 0.3 mM to about 1.2 mM, or more preferably about 0.5 mM to about 1.0 mM.

The treatment solution may additionally include an antifoam agent, which is particularly beneficial for crude samples such as saliva that tend to foam. The antifoaming agent is preferably included in an amount of about 0.001% to about 0.008% w/v, or more preferably about 0.0015% to about 0.004% w/v. The antifoaming agent may be formulated with silicon and nonionic emulsifiers, such as the antifoam agent SE-15.

As mentioned above, the treatment solution is formulated for mixing directly with a crude biological sample. The treatment composition can be formulated for mixing with the crude biological sample at a treatment composition to sample ratio of about 0.5:1 to about 4:1, or at a ratio of about 1:1 to about 2:1, with component amounts of the treatment composition being scaled accordingly for other mixture ratios. In other words, the concentrations of the components of the treatment composition described herein assume a mixing ratio within the foregoing ranges, but where other mixing ratios are utilized, the concentrations may be scaled accordingly.

The mixing ratio may also depend on the collection method of the sample. For example, where the treatment solution is mixed directly with a liquid sample (e.g., saliva, blood, urine, etc.), it will typically be mixed at a ratio closer to about 1:1 (e.g., 0.5:1 to 2:1), whereas when the treatment solution is mixed with a swab (or similar collection device) to resuspend material collected on the swab, the relative amount of treatment solution may be increased and the ratio will typically be higher, such as about 2:1 (e.g., 1.5:1 to 4:1).

The treatment solution is preferably formulated such that the pH is about 7 or greater, such as about 7.2 to about 8.

After mixing the treatment solution with the sample, the mixture may be utilized for subsequent analysis and/or detection of nucleic acids within the sample. Often, the analysis will include polymerase chain reaction (PCR). Where the target nucleic acid is RNA, such as in the case of RNA viruses including SARS-CoV-2, the subsequent analysis may involve reverse transcription PCR (RT-PCR).

The treatment solution is formulated to promote stability of the solution-sample mixture. For example, mixtures can remain stable at room temperature for at least about 96 hours. The term “stable”, as used in this context, means that the solution-sample mixture may be subsequently processed with zero or negligible (e.g., less than 10%) loss of sensitivity to nucleic acid detection as compared to otherwise similar solution-sample mixtures that are processed without such a waiting period.

The treatment solutions described herein beneficially provide one or more of: (i) stabilization of the crude biological sample when mixed; (ii) inactivation of at least one virus and/or microorganism within the crude biological sample; (iii) lysis of animal cells and/or the at least one virus and/or microorganism within the crude biological sample; (iv) reduction in viscosity of the crude biological sample; (v) improving accessibility to viral and/or other microorganism nucleic acids within the crude biological sample; and (vi) preserving integrity of nucleic acids within the crude biological sample without extraction or purification of the nucleic acids. Treatment solution formulations can more beneficially provide two or more, or three or more, or even all of the foregoing functions.

### Sample Processing Methods for Detecting a Target Nucleic Acid in a Sample

Treatment solutions such as those described above can be used to process a crude biological sample containing or suspected of containing a target nucleic acid. In one embodiment, a method includes the steps of: (a) contacting the biological sample with a treatment solution comprising a protease component to form a mixture; (b) inactivating the protease component in the mixture of (a); and (c) performing an analysis of the target nucleic acid.

The mixing of step (a) may include any of the treatment solutions described in the above section. As mentioned above, the treatment solution may be mixed with the crude biological sample at a treatment solution to sample ratio of about 0.5:1 to about 4:1, or at a ratio of about 1:1 to about 2:1, with component amounts of the treatment composition being scaled accordingly for other mixture ratios. The biological sample thus typically makes up about 10% to 60% of the volume of the mixture of step (a). Following formation of the mixture of step (a), but prior to step (b), the mixture may be stored and/or shipped for a period of time. This period may have a duration of up to about 96 hours (assuming room temperature conditions or similar), and the mixture beneficially remains stable throughout this period. The period may be longer than about 96 hours in certain situations, such as where refrigeration and/or other storage procedures are implemented.

The biological sample may include one or more of a saliva sample, buccal sample, nasal sample, nasal pharyngeal sample, blood sample, urine sample, fecal sample, or semen sample. The biological sample may be mixed directly with the treatment solution, or may be a resuspension of sample previously obtained using a swab or other sample collection device. The volume of such a resuspension may depend on the type of sample and particular application protocols, but is preferably a small resuspension volume of about 0.1 ml to about 1 ml.

Inactivation of the protease component in step (b) may involve temperature treatment, the addition of a protease inhibitor component, or both. The temperature treatment may include sequentially treating the mixture at a first temperature and then a second, different temperature. The first and second temperatures preferably differ by at least about 15° C. The first temperature may include a temperature between 20° to 70° C. and the second temperature may include a higher temperature. The first temperature may be room temperature, or about 25° C., for example. The second temperature may be varied according to the first. For example, the second temperature may typically lie between about 85° to 100° C., but may be set at a lower temperature when the first temperature is high enough to compensate.

The duration of incubation at the first temperature may depend on the first temperature, with the duration being longer for relatively lower temperatures and shorter for relatively higher temperatures. In other words, the higher the first temperature is, the less time needed before moving to the second temperature. The duration of incubation at the first and second temperatures may be about 2 minutes each. Incubation at the second temperature is preferably no longer than about 15 minutes.

The temperature treatment may further include incubating the mixture at a third temperature for a third time interval prior to performing step (c). The third temperature may be between 2° to 8° C. The third temperature may be for at least one minute, though the mixture may be stored for up to 24 hours at the third temperature prior to performing step (c).

The temperature treatment may further include incubating the mixture at a fourth temperature prior to step (c). The first and fourth temperatures may be substantially the same. For example, the fourth temperature may be about 25° C. or room temperature. The mixture is beneficially stable at the fourth temperature for at least 96 hours.

In embodiments where a protease inhibitor is added to the mixture in step (b), the protease inhibitor may include a mixture with a plurality of protease inhibitors, also referred to herein as a “protease inhibitor cocktail”. A preferred protease inhibitor cocktail is sold under the name HALT, and includes six different inhibitors: AEBSF (1 mM), aprotinin (800 nM), bestatin (50 μM), E64 (15 μM), leupeptin (20 μM), and pepstatin A (10 μM). One or more of such inhibitors may be utilized independently or in any combination, with preferred embodiments using multiple protease inhibitors.

The analysis of step (c) may include amplifying one or more target nucleic acids within the biological sample. The described methods beneficially enable more efficient amplification of the target nucleic acid, resulting in a lower Ct value, as compared to otherwise similar samples in water and/or TE buffer. In some embodiments, multiple different nucleic acids are amplified, such as in a multiplex reaction. For example, a first target nucleic acid may be from a target virus or microbe, while a second target nucleic acid is from the organism from which the biological sample is obtained (e.g., from a patient). The second target nucleic acid may be an RNase P nucleic acid, for example. In other embodiments, a first target nucleic acid may be from a target virus or microbe, while a second target nucleic acid is an external positive control nucleic acid, such as bacteriophage MS2 control nucleic acid.

The amplification of the target nucleic acid(s) may be via PCR. Those skilled in the art will understand that various different types of PCR processes may be utilized, including RT-PCR where appropriate, such as where a target is a SARS-CoV-2 nucleic acid. The PCR may be a quantitative PCR (qPCR) or endpoint PCR, enabling quantification the amount of the target nucleic acid present in the crude biological sample.

Steps (a) through (c) may be performed in a single reaction vessel or multiple reaction vessels. For example, steps (a) and (b) may be performed in a first reaction vessel or tube while step (c) is performed in a second reaction vessel or tube. In some embodiments, an aliquot of the mixture from (b) is transferred to the second reaction vessel and further diluted prior to performing step (c). The aliquot of the mixture from (b) may be mixed with one or more PCR reagents in the second reaction vessel.

The processing method may further include the step of diagnosing an infection in an organism from which the biological sample was obtained based on the analysis/detection of step (c). The organism may be a mammal, including a human. The infection may be associated with a virus, including an enveloped virus such as a coronavirus. The methods described herein are particularly beneficial for detecting SARS-CoV-2 within a crude sample.

In some embodiments, a plurality of separate samples from different individuals are pooled together to form a multi-individual biological sample, and the multi-individual biological sample is then utilized in steps (a) through (c) (and optionally any of the other additional steps described herein). Pooling is beneficial in certain situations where the target nucleic acid is prevalent at a level where mixing of samples can allow faster and/or more efficient screening of multiple samples. In some embodiments, 2, 3, 4, or 5 individual samples are pooled together prior to treatment, such as in step (a) or prior to step (a).

Where the target is detected in a particular multi-individual biological sample, practitioners can then work backwards to individually test the plurality of samples mixed together to make the particular multi-individual biological sample. Typically, enough sample is collected from each of the donating individuals at the time of collection so as to have enough set aside for individual sample testing if the target is detected within the corresponding pooled sample. Pooling thus increases overall testing efficiency where the efforts required to backtrack in the case of detecting the target in a pooled sample are offset by the efficiency gains made from pooled samples with negative results (which indicates that none of the samples that make up the pooled sample included the target).

In some embodiments, the entire method may be performed at a point of service. For example, all of steps (a) through (c) may be performed at the same general location without the need for shipping or transport of the sample to separate locations. That is, while some movement of the sample may be necessary (e.g., from room to room within a building), the sample need not be packaged and shipped to a distant location. Preferably, sample collection is also performed at the same location. For example, the sample can be collected from a subject and then immediately or relatively soon thereafter processed in steps (a) through (c). The point of service location may be a testing site, medical facility (e.g., hospital, clinic), school, event center, or transportation center such as an airport, stadium, or arena.

In some embodiments, a method for processing a biological sample containing or suspected of containing a target nucleic acid is carried out according to one or more steps of the following exemplary protocol. Raw saliva samples are optionally heated at 95° C. for about 5 minutes, then allowed to cool to room temperature. For the required number of samples, 20 μL of treatment solution is added to each well of a well plate (e.g., 96 well plate). Each saliva sample is vortexed for at least 10 seconds, or until the sample appears homogenous. 20 μL of the saliva sample(s) are then added to the wells containing the treatment solution, followed by pipetting up and down to mix. The plate is then sealed with clear adhesive film, then vortexed on all sides along the skirt of the plate for about 5 seconds per side. The plate is then centrifuged for about 30 seconds at 200×g to collect the samples at the bottom of the wells. The plate is then heated in a thermal cycler with the following conditions: 62° C. for 5 minutes, 92° C. for 5 minutes, and then hold at 4° C. until ready for further analysis. The sealed plate may be stored (e.g., for up to about 24 hours) until used for PCR or other analysis. Preferably, about 7 μL of prepared sample is used in a 10 μL PCR, such as a real-time RT-PCR.

### Kits for Detecting a Target Nucleic Acid in a Sample

Although the treatment solutions described herein may be provided as stand-alone compositions (e.g., provided in a suitable container such as a tube, vial, or the like), some embodiments relate to kits that include a treatment solution and one or more additional components. When provided as a stand-alone composition, a treatment solution may be utilized as desired by a user with one or more additional components as desired for enabling processing of the sample and detecting target nucleic acids within the sample. A kit for detecting a target nucleic acid in a sample can include a treatment solution according to the formulations described above, along with one or more additional components that enable processing of the sample for detecting the target nucleic acid within the sample. In one embodiment a kit includes the treatment solution and one or both of a PCR reagent mixture and a sample collection device.

The PCR reagent mixture may include at least one primer and/or at least one probe. The PCR reagent mixture may include primers corresponding to a plurality of target nucleic acids.

Exemplary primers are described in more detail below. The PCR reagent mixture may also comprise any other components necessary for carrying out PCR reactions, such as RT-qPCR reactions, including TaqMan Fast Virus 1-Step Master Mix (sold by Thermo Fisher Scientific under Catalog No. 44444432) or TaqPath 1-Step RT-qPCR Master Mix (sold by Thermo Fisher Scientific under Catalog No. A15299). In some embodiments, the kit includes primers, probes, and master mix sufficient to constitute a reaction mixture supporting singleplex or multiplex amplification of one or more SARS-CoV-2 regions encoding the N protein, the S protein and/or ORF1ab protein. In some embodiments, at least one of the components of the kit is dried or freeze dried (e.g., lyophilized).

Where a sample collection device is included, it may be in the form of a dry swab collection device, such as a spun polyester swab collection device, a flocked swab non-breakable collection device, or a flocked swab, breakable collection device. The collection device may be in the form of a saliva collection container, such as a tube or vial. The kit may be configured for enabling a user to self-collect the biological sample.

### Additional Nucleic Acid Amplification Details

Certain embodiments described herein involve the detection and identification of one or more target nucleic acids in the sample, which may be single stranded or double stranded and may be of any size. Much of the following relates to detection of targets with RNA-based genomes, and in particular to detection of SARS-CoV-2. The skilled person will understand, however, in light of this disclosure, that amplification procedures may be modified as appropriate for other types of microbes and/or viruses using appropriate components (primers, probes, etc.) and processes.

As described herein, the target sequence may be associated with the N protein, the ORFlab protein, and/or the S protein of the SARS-CoV-2 genome. In some embodiments, the primer and/or probe sequences described by the United States Centers for Disease Control and Prevention (CDC) may be utilized (https://www.cdc.gov/coronavirus/2019-ncov/lab/rt-pcr-panel-primer-probes.html). For example, assays described herein may include one or more primers and/or probes shown in Table 1A.

TABLE 1A
CDC 2019-Novel Coronavirus (2019-nCoV) Real-time RT-PCR Primers and Probes

| Name/Description | SEQ ID NO | Labels^{1, 2} | Final Conc.* |
|---|---|---|---|
| 2019-nCoV_N1 Forward Primer | SEQ ID NO: 1311 | None | 500 nM |
| 2019-nCoV_N1 Reverse Primer | SEQ ID NO: 1312 | None | 500 nM |
| 2019-nCoV_N1 Probe | SEQ ID NO: 1313 | FAM, BHQ-1 | 125 nM |
| 2019-nCoV_N1 Probe | SEQ ID NO: 1313 | FAM, ZEN, 3IABkFQ | 125 nM |
| 2019-nCoV_N2 Forward Primer | SEQ ID NO: 1314 | None | 500 nM |
| 2019-nCoV_N2 Reverse Primer | SEQ ID NO: 1315 | None | 500 nM |
| 2019-nCoV_N2 Probe | SEQ ID NO: 1316 | FAM, BHQ-1 | 125 nM |
| 2019-nCoV_N2 Probe | SEQ ID NO: 1316 | FAM, ZEN, 3IABkFQ | 125 nM |
| RNase P Forward Primer | SEQ ID NO: 1317 | None | 500 nM |
| RNase P Reverse Primer | SEQ ID NO: 1318 | None | 500 nM |
| RNase P Probe | SEQ ID NO: 1319 | FAM, BHQ-1 | 125 nM |
| RNase P Probe | SEQ ID NO: 1319 | FAM, BHQ-1 | 125 nM |
| RNase P Probe | SEQ ID NO: 1319 | FAM, ZEN, 3IABkFQ | 125 nM |

¹The labels shown are exemplary to some embodiments of the compositions, reactions mixtures, kits, or methods described herein and do not limit other possible labels, including various fluorophores and quenchers, contemplated for use in the primers and probes described herein.
²TaqMan ® probes are labeled at the 5′-end with the reporter molecule 6-carboxyfluorescein (FAM) and with the quencher, Black Hole Quencher 1 (BHQ-1) (Biosearch Technologies, Inc., Novato, CA) at the 3′-end. TaqMan ® probes can also be labeled at the 5′-end with the reporter molecule 6-carboxyfluorescein (FAM) and with a double quencher, ZEN ™ Internal Quencher positioned between the ninth (9th) and tenth (10th) nucleotide base in the oligonucleotide sequence and Iowa Black ® FQ (3IABkFQ) located at the 3′-end (Integrated DNA Technologies, Coralville, IA).
*The final concentrations shown are exemplary to some embodiments of the compositions, reactions mixtures, kits, or methods described herein and are in no way meant to limit other possible concentrations or concentration ranges for use in the compositions, reactions mixtures, kits, or methods contemplated herein.

In some other embodiments, the primers and probes provided in SEQ ID NO:4-SEQ ID NO: 1300 can be used to amplify and/or analyze one or more specific target sequences present in one or more target genome(s), as described herein. The primer and probe sequences provided in SEQ ID NO:4-SEQ ID NO:1300 are targeted to regions of the SARS-CoV-2 genome, human influenza type A or type B genomes, regions of the RSV type A or type B genomes, or control sequences such as MS2 Phage and RNase P. The primer and probe sequences described herein need not have 100% homology to their targets to be effective, though in some embodiments, homology is substantially 100%. In some embodiments, one or more of the disclosed primer and/or probe sequences have a homology to their respective target of at least about 50%, at least about 60%, at least about 70%, at least about 80%, at least about 85%, at least about 90%, at least about 95%, at least about 97%, at least about 98%, at least about 99%, or up to substantially 100%. Some combinations of primers and/or probes may include primers and/or probes each with different homologies to their respective targets, and the homologies may be, for example, within a range with endpoints defined by any two of the foregoing values.

In particular, kits may contain one or more of the forward primers, reverse primers, and/or probes for detecting target nucleic acid sequences in one or more of the SARS-CoV-2, influenza A, influenza B, RSV A, or RSV B genomes, as well as control sequences, such as disclosed in SEQ ID NO:4-SEQ ID NO:257 (forward primers), SEQ ID NO:267-SEQ ID NO: 510 (reverse primers), and SEQ ID NO:520-SEQ ID NO:1300 (probes). The amplified products (“amplicons”) can be detected and/or analyzed using any suitable method and on any suitable platform readily known to those having skill in the art.

Polymerase chain reaction (PCR) and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as, e.g., loop-mediated isothermal amplification (“LAMP”), and other isothermal methods, such as those listed in Table 1B, may require less or less extensive thermal cycling than does PCR, or require no thermal cycling. Such isothermal amplification methods are also contemplated for use with the assay compositions, reaction mixtures, kits described herein.

TABLE 1B
Summary of optional isothermal amplification methods.

| | |
|---|---|
| NASBA | Nucleic acid sequence-based amplification (NASBA) is a |
| | method used to amplify RNA. |
| LAMP | Loop-mediated isothermal amplification (LAMP) is a single |
| | tube technique for the amplification of DNA. It typically |
| | uses 4-6 primers, which form loop structures to facilitate |
| | subsequent rounds of amplification. |
| HDA | Helicase-dependent amplification (HDA) uses the double- |
| | stranded DNA unwinding activity of a helicase to separate |
| | strands for in vitro DNA amplification at constant temperature. |
| RCA | Rolling circle amplification (RCA) starts from a circular |
| | DNA template and a short DNA or RNA primer to form a long |
| | single stranded molecule. |
| MDA | Multiple displacement amplification (MDA) is a technique |
| | that initiates when multiple random primers anneal to the |
| | DNA template and the polymerase amplifies DNA at constant |
| | temperature. |
| WGA | When MDA is used to amplify DNA from a whole genome of a |
| | cell it is called whole genome amplification (WGA). (Other |
| | methods of WGA include MALBAC, LIANTI, DOP-PCR.) |
| RPA | Recombinase polymerase amplification (RPA) is a low |
| | temperature DNA and RNA amplification technique. |

Methods of performing amplification, including those in Table 1B are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

SARS-CoV-2 has a single-stranded positive-sense RNA genome. Other viruses, such as Influenza A, Influenza B, RSV A, and RSV B also have RNA-based genomes. In some embodiments, therefore, the amplification reaction (e.g., LAMP or PCR) can be combined with a reverse transcription (RT) reaction, such as in RT-LAMP or RT-PCR to convert the RNA genome to a cDNA template. The cDNA template is then used to create amplicons of the target sequences in the subsequent amplification reactions.

In some embodiments, the amplifying step can include performing qPCR, as that term is defined herein. qPCR is a sensitive and specific method for detecting and optionally quantifying amounts of starting nucleic acid template (e.g., coronaviral nucleic acid) in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Suitable labels are described herein. In some embodiments, the dye-quencher combinations are used, such as those described in the Examples. It should be appreciated that qPCR and RT-qPCR methods are known to those having skill in the art. Nevertheless, particular embodiments are provided in the Examples and provide further details regarding qPCR as well as related compositions and methods of use thereof.

The reaction vessel or volume can optionally include a tube, channel, well, cavity, site or feature on a surface, or alternatively a droplet (e.g., a microdroplet or nanodroplet) that may be deposited onto a surface or into a surface well or cavity, or suspended within (or partially bounded by) a fluid stream. In some embodiments, the reaction volume includes one or more droplets arrayed on a surface or present in an emulsion. The reaction volumes can optionally be formed by fusion of multiple pre-reaction volumes containing different components of an amplification reaction. For example, pre-reaction volumes containing one or more primers can be fused with pre-reaction volumes containing human nucleic acid samples and/or polymerase enzymes, nucleotides, and buffer. In some embodiments involving performing qPCR reactions in array format, a surface contains multiple grooves, channels, wells, cavities, sites, or features defining a reaction volume containing one or more amplification reagents (e.g., primers, probes, buffer, polymerase, nucleotides, and the like). In some array-formatted singleplex embodiments, the reaction volume within the selected tubes, grooves, channels, wells, cavities, sites, or features contains only a single forward primer sequence and a single reverse primer sequence. Optionally, a probe sequence is also included in the singleplex reaction volume.

In some array-formatted multiplex embodiments, the reaction volume within the selected tubes, grooves, channels, wells, cavities, sites, or features contains multiple (e.g., 2, 3, 4, 5, 6, etc.) forward primer sequences and multiple reverse primer sequences. Optionally, one or more probe sequences is also included in the multiplex reaction volume. For instance, exemplary methods for polymerizing and/or amplifying and detecting nucleic acids suitable for use as described herein are commercially available as TaqMan assays (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety).

TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. The oligonucleotide probe typically includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

For example, a singleplex or multiplex qPCR can include a single TaqMan dye associated with a locus-specific primer or multiple TaqMan dyes respectively associated with a plurality of loci in a multiplex format. As a non-limiting example, a 4-plex reaction can include FAM (emission peak ˜517 nm), VIC (emission peak ˜551 nm), ABY (emission peak ˜580 nm), and JUN (emission peak ˜617 nm) dyes, each dye being associated with a different target sequence and each dye being quenched by QSY, can allow up to 4 targets to be amplified and tracked real-time within a single reaction vessel. These aforementioned reporter dyes are optimized to work together with minimal spectral overlap for improved performance. These dyes can additionally be combined with Mustang Purple (emission peak ˜654 nm) for use monitoring fluorescence of a control or for use in a non-emission-spectrum-overlapping 5-plex assay. In addition, the QSY quencher is fully compatible with probes that have minor-groove binder quenchers.

Detector probes may be associated with alternative quenchers, including without limitation, dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy5.

It should be appreciated that when using more than one detectable label, particularly in a multiplex format, each detectable label should differ in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′,7′-dimethoxy fluorescein (JOE)); 6-carboxy-1,4-dichloro-2′,7′-dichloro fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalama1), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl-rhodamine), Tetramethylrhodamine (TRITC), WT), Texas Red, Texas Red-X, among others as would be known to those of skill in the art.

Other detectable labels may also be used. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30 (9): e37), Hayashi et al. (Nucleic Acids Res. 1989 May 11; 17 (9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

The genetic sequence of SARS-CoV-2 is available as GenBank: MN908947.3 and describes a positive-sense, single-stranded RNA genome of 29,844 base pairs. Initial genetic characterizations of SARS-CoV-2 identified three coronaviruses having close homology to SARS-CoV-2, namely Bat-SL-CoVZC45, Bat-SL-CoVZXC21, and SARS-CoVGZ02. There are at least three genetic regions with significant variability between SARS-CoV-2 and the other related viruses, specifically within the viral genes encoding the ORFlab protein (SEQ ID NO:1 bp 1 corresponds to bp 1000 of MN908947), the S protein (SEQ ID NO:2; bp 1 corresponds to bp 21564 of MN908947), and the N protein (SEQ ID NO:3; bp 1 corresponds to bp 28275 of MN908947). The region comprising the coding sequence for the ORFlab protein is between base pairs 1000-3000 of the SARS-CoV-2 genome; this sequence corresponds to SEQ ID NO:1. The 2,000 base pair region of the SARS-CoV-2 genome that includes the coding sequence for the S protein is between base pairs 21,564-23,564; this sequence corresponds to SEQ ID NO:2. Finally, the 1,283 base pair region of the SARS-CoV-2 genome that includes the coding sequence for the N protein is between base pairs 28,275-29,558; this sequence corresponds to SEQ ID NO:3.

In some embodiments, emerging variants of SARS-CoV-2 are detectable even if such variants include mutations in one or more of the target regions described above (i.e., ORF1ab protein, S protein, or N protein regions). By looking at multiple target regions within the SARS-CoV-2 genome, accurate detection is achievable even in situations where mutations are significant enough to lead to a negative test result in one (or even two) of the target regions. For example, newly emerging variant B.1.1.7 (often referred to as “the UK variant” or “the alpha variant”) has an unusual number of mutations associated with the S protein region. These mutations are substantial enough that some test components and protocols designed for earlier SARS-CoV-2 variants will show a negative result for the S protein region. However, the built-in redundancy of looking at multiple regions ensures that the overall test is still capable of detecting the SARS-CoV-2 variant B.1.1.7 (based on positive ORF1ab and/or N protein region detection) without significant effects on the overall accuracy of the test. In another example, the 501Y.V2 variant (discovered in South Africa) has not been found to affect detection of the S protein region or any of the other tested regions described herein. Nevertheless, the robustness and redundancy of embodiments that target multiple regions of the SARS-CoV-2 genome limit the risk that these variants, or others that emerge in the future, will significantly impact the overall accuracy of SARS-CoV-2 detection.

In some embodiments, amplification of RNA viral genomes is achieved by performing reverse transcription followed by amplification of at least a portion of the resultant cDNA. Suitable methods are known in the art and include, for example, RT-PCR or RT-LAMP methods where the target sequence (e.g., viral RNA genome) is reverse transcribed to form a first cDNA strand, which is then copied in a template-dependent fashion to form a double stranded DNA sequence. The target sequence is then amplified from this double-stranded cDNA.

In some embodiments, the RT-PCR may be a one-step procedure using one or more primers and one or more probes as described herein. In some embodiments, the RT-PCR may be carried out in a single reaction tube, reaction vessel (e.g., “single-tube” or “1-tube” or “single-vessel” reaction). In some embodiments, the RT-PCR may be carried out in a multi-site reaction vessel, such as a multi-well plate or array. In some embodiments, RT and PCR are performed in the same reaction vessel or reaction site, such as in 1-step or 1-tube RT-qPCR. Suitable exemplary RTs can include, for instance, a Moloney Murine Leukemia Virus (M-MLV) Reverse transcriptase, SuperScript Reverse Transcriptases (Thermo Fisher Scientific), SuperScript IV Reverse Transcriptases (Thermo Fisher Scientific), or Maxima Reverse Transcriptases (Thermo Fisher Scientific), or modified forms of any such RTs, including hybrid or fusion RTs.

In some embodiments, only a single RT-qPCR assay (consisting of a given forward primer and a given reverse primer sequence) is included within a reaction vessel or volume, a reaction mode referred to as “singleplex” herein. Optionally, the singleplex qPCR assay can also include a single probe sequence in addition to the forward primer sequence and the reverse primer sequence. The probe sequence can be a hydrolysis probe sequence. In other embodiments, the probe sequence can be a molecular beacon probe sequence. Optionally, the probe includes an MGB (minor groove binding protein).

In some embodiments, the disclosed compositions and methods can be used in multiplex format, wherein two or more qPCR assays, each capable of amplifying or detecting a different target sequence, are present in a single reaction volume. In some embodiments, different assays in the same reaction volume will cause a corresponding different amplification product to be generated when the reaction volume is subjected to appropriate amplification conditions and multiple amplicons may be formed in the same reaction volume. The different amplification products can be produced simultaneously when the reaction volume is subjected to amplification conditions; alternatively, different amplification products may be produced serially or consecutively. For example, some assay reaction products may take longer to appear than others due to initial starting concentration of template or may benefit from different reaction conditions for optimal production.

In some embodiments, different assay products can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety. In some embodiments, specific combinations of labels are used to differentiate between different pathogens, strains, and/or types of pathogens. For example, different respiratory pathogens or viruses may be differentiated from one another using different labels specific to each pathogen or virus such that the label is detectable only in the presence—and amplification—of the pathogen- or viral-specific nucleic acid sequence.

In some array-based embodiments, two or more different qPCR assays (each containing a forward primer, a reverse primer and optionally a probe) are present in a single well, cavity, site or feature of the array and products of each assay can be independently detected. For example, different assay products may be discriminated optically (e.g., using different labels present as components of each assay) or using some other suitable method, including as described in U.S. Patent Publication No. 2019/0002963. In some embodiments, at least one primer of each assay contains an optically detectable label that can be discriminated from the optical label of at least one other assay. For the purposes of this disclosure, a PCR assay, which for the sake of clarity is inclusive of any polymerase-driven amplification reaction disclosed herein (e.g., qPCR and RT-qPCR), is considered different from another PCR assay if the respective amplicons differ in nucleic acid sequence by at least one nucleotide.

In some embodiments, the reverse transcription and/or nucleic acid amplification assays as described herein are performed using a real-time quantitative PCR (qPCR) instrument, including for example a QuantStudio Real-Time PCR system, such as the QuantStudio 5 RealTime PCR System (QS5) and QuantStudio 12K Flex System (QS12K), or a 7500 Real-Time PCR system, such as the 7500 Fast Dx system, from Thermo Fisher Scientific.

In some embodiments, the primers and/or probes associated with SEQ ID NO:4-SEQ ID NO: 1300 may further comprise a fluorescent or other detectable label and/or a quencher or minor groove binder, such as those described above. As a non-limiting example, said primers and/or probes can be associated with FAM, ABY, VIC, or JUN as detectable labels and QSY as a quencher. In some embodiments of multiplex assay formats described herein, various SARS-CoV-2 genomic regions are detected, including assays for detecting the coding regions of ORF1ab (e.g., FAM-labeled), N Protein (e.g., VIC-labeled), and S Protein (e.g., ABY-labeled). As described above, one or more labelled primers may be used, in addition to or as an alternative to labelled probes, for detecting one or more target nucleic acids. Thus, in some embodiments, no probes are utilized.

Optionally, in some embodiments, a control (e.g., JUN-labeled), such as bacteriophage MS2 or RNase P control, is included in the kit, array, reaction mixture, etc. comprising the multiplex assay. If the positive control sequence is an endogenously-derived control, such as RNase P, the presence of patient-derived nucleic acid (e.g., genomic DNA coding for RNase P, RNase P RNA, and/or reverse transcribed RNase P transcript), can be used as the template for an RNase P qPCR assay. Exemplary primers and probes for such an RNase P qPCR positive control can include SEQ ID NO: 1317-SEQ ID NO:1319, although those having skill in the art should appreciate that other RNase-P-specific primers and/or probes could be used. If the positive control sequence is an exogenously-derived control, such as a component of the MS2 bacteriophage, a known or predetermined concentration of template nucleic acid is added to the reaction volume to serve as the requisite template for an MS2 qPCR assay.

It should be appreciated that although particular examples are provided above indicating a given fluorophore associated with detection of a given viral sequence, the probes can be modified to include a functionally similar fluorophore described herein or as otherwise known in the art. Further, quenchers, such as QSY, can be included in any of the foregoing examples, and the detectable label and/or quencher can be selected based on the singleplex or multiplex requirements of the given qPCR assay in accordance with the constraints and considerations discussed above or otherwise understood by those having skill in the art.

### Abbreviated List of Defined Terms

To assist in understanding the scope and content of the foregoing and forthcoming written description and appended claims, a select few terms are defined directly below.

The SARS-CoV-2 virus, also known as 2019-nCoV, is associated with the human respiratory disease COVID-19. The virus isolated from early cases of COVID-19 was provisionally named 2019-nCoV, and the Coronavirus Study Group of the International Committee on Taxonomy of Viruses subsequently designated 2019-nCoV as SARS-CoV-2. For the purposes of this disclosure, the term “SARS-CoV-2” and “2019-nCoV” are considered to refer to the same virus and may be used interchangeably to refer to the etiologic agent for COVID-19. As used herein, these terms are also inclusive of separate variants of SARS-CoV-2, including variant B.1.1.7, variant 501Y.V2, variant B.1.617.2 (“the Delta variant”), variant B.1.1.529 (“the Omicron variant”), and other variants that may emerge in the future.

As used herein the term “crude biological sample” refers to a biological sample that has not been subjected to pre-processing steps intended to extract, isolate, or purify particular subcomponents (such as nucleic acids) of the sample from the remainder of the sample. Crude biological samples can include a saliva sample, buccal sample, nasal sample, nasal pharyngeal sample, blood sample, urine sample, or semen sample, for example. In some embodiments, crude biological samples can be diluted by addition of a preservative solution (such as storage buffer), water, and the like.

As used herein the terms “Ct” and “cycle threshold” (also sometimes referred to as a C_{q} value) refer to the time at which fluorescence intensity is greater than background fluorescence. They are characterized by the point in time (or PCR cycle) where the target amplification is first detected. Consequently, the greater the quantity of target DNA in the starting material, the faster a significant increase in fluorescent signal will appear, yielding a lower Ct.

As used herein, the term “kit” refers to any delivery system for delivering materials. In the context of reaction assays, such delivery systems include systems that allow for the storage, transport, or delivery of reaction reagents (e.g., oligonucleotides, enzymes, primer set(s), etc. in the appropriate containers) and/or supporting materials (e.g., buffers, written instructions for performing the assay etc.) from one location to another. For example, kits can include one or more enclosures (e.g., boxes) containing the relevant reaction reagents and/or supporting materials. As used herein, the term “fragmented kit” refers to a delivery system comprising two or more separate containers that each contain a sub-portion of the total kit components. The containers may be delivered to the intended recipient together or separately. For example, a first container may contain an enzyme for use in an assay, while a second container contains oligonucleotides. Indeed, any delivery system comprising two or more separate containers that each contains a sub-portion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits. Components described herein may be combined with one another and provided as such a combined kit or fragmented kit, or alternatively each component may be provided separately and utilized as desired by a user. For example, a treatment solution as described herein may be included in a kit or may be provided as a “stand-alone” item (in an appropriate container) for use as desired by the user.

The term “primer,” as used herein, is intended to encompass those sequence-specific oligonucleotides used in amplification reactions (e.g., PCR, qPCR, RT-qPCR, or similar), where the primer binds to a complementary template sequence and provides a free 3′-end for a nucleic acid polymerase to synthesize a new strand from the bound template. Primers may optionally be labelled (e.g., with one or more detectable labels, including a fluorescent label) to enable the detection and/or concentration of associated amplicons.

As used herein, the term “probe” includes those sequence-specific oligonucleotides (whether DNA or RNA) that function to detect the presence or absence of target nucleic acid present in a sample or reaction. A probe, therefore, may include an oligonucleotide portion and a detectable label associated therewith.

As used herein, the terms “real-time PCR” or “quantitative real-time PCR” or “qPCR” refer to the measurable amplification of nucleic acids via PCR in real time, typically by monitoring detectable probes (typically fluorescent probes) in the reaction volume and enabling the optional quantitation of the PCR product. The terms “real-time” and “real-time continuous” are interchangeable and refer to a method where data collection occurs through periodic monitoring during the course of the amplification reaction. Thus, real-time methods combine amplification and detection into a single step. It should be appreciated that the data collection may occur through periodic monitoring during the course of PCR while the analysis of such data may occur later in time.

The terms “reverse transcription PCR” or simply “RT-PCR” are intended to include those PCR methods that first transcribe an RNA template (such as a viral RNA genomic template) into complementary DNA (cDNA) using an RNA-dependent DNA polymerase generally referred to as a reverse transcriptase. The cDNA is then used by any of the DNA-dependent DNA polymerases commonly used in PCR methods as a template for PCR amplification of the target nucleic acid sequence. For ease of use within the specification, the terms “RT-PCR” and “RT-qPCR” may be used interchangeably, as it is understood by those having skill in the art that methods and reagents for monitoring amplicon production at the endpoint, such as is done in traditional PCR methods, can be adjusted such that amplicon production can be monitored during and/or between thermal cycles of PCR, such as is done in traditional qPCR methods.

Further, it should be appreciated that when the term “qPCR” is used herein, it does not necessarily exclude methods and/or kits that include an initial reverse transcription step. As such, any indication within the specification of a “qPCR” method, kit, array, and/or assay for performing qPCR is understood to include the same or similar method, kit, array, and/or assay having an initial reverse transcription step with any attendant reagents (e.g., reverse transcriptase, buffers, dNTPs, salts, etc.).

Unless defined otherwise, all technical and scientific terms used herein have the same meaning as commonly understood by one of ordinary skill in the art to which the present disclosure pertains.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively.

It will be noted that, as used in this specification and the appended claims, the singular forms “a,” “an” and “the” include plural referents unless the context clearly dictates otherwise. Thus, for example, reference to a singular referent (e.g., “widget”) includes one, two, or more referents. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

To facilitate understanding, like reference numerals (i.e., like numbering of components and/or elements) have been used, where possible, to designate like elements common to the figures. Specifically, in the exemplary embodiments illustrated in the figures, like structures, or structures with like functions, will be provided with similar reference designations, where possible. Specific language will be used herein to describe the exemplary embodiments. Nevertheless, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

Any headings used herein are for organizational purposes only and are not meant to be used to limit the scope of the description or the claims.

Various aspects of the present disclosure can be illustrated by describing components that are bound, coupled, attached, connected, and/or joined together. As used herein, the terms “bound,” “coupled”, “attached”, “connected,” and/or “joined” are used to indicate either a direct association between two components or, where appropriate, an indirect association with one another through intervening or intermediate components. In contrast, when a component is referred to as being “directly bound,” “directly coupled”, “directly attached”, “directly connected,” and/or “directly joined” to another component, no intervening elements are present or contemplated. Furthermore, binding, coupling, attaching, connecting, and/or joining can comprise mechanical and/or chemical association.

### EXAMPLES

### Example 1: Effect of Surfactant Solutions on Virus Genomic RNA Reverse Transcription and Amplification

A screening method was devised for discovering surfactants that are capable of disrupting the SARS-CoV-2 virus and making the viral RNA genome readily accessible for enzymatic reactions such as reverse transcription at temperatures below those known to result in virus inactivation. Heat inactivation conditions that are known in the art for enveloped viruses include incubating samples at 56° C. for 30 minutes or 65° C. for 15 minutes. In this example, a sample of chemically-inactivated SARS-Cov-2 virus particles (Zeptometrix, Part No. NATSARS (COV2)-ST) was first diluted to 27 particles per microliter in Tris-EDTA buffer (10 mM Tris, pH 7.5 and 0.75 mM EDTA, pH 8.0). 15 μL of diluted sample was then combined with 15 μL of a 2× surfactant treatment solution as described in Table 2 and mixed thoroughly by touch vortexing for 10 seconds.

TABLE 2
Surfactant Treatment Solutions
Surfactant Treatment Solution

| | | | | Critical Micelle |
|---|---|---|---|---|
| | | | Concentration | Concentration |
| Surfactant | Vendor | Part No. | (2X) | (CMC), w/v % |
| Triton X-100 | Sigma | 93443 | 0.20% | 0.056% |
| Tween 20 | Thermo | 28320 | 0.04% | 0.007% |
| | Fisher | | | |
| | Scientific | | | |
| N,N Dimethydodecylamine- | Sigma | 40236 | 0.01% | 0.034% |
| N-oxide (LDAO) | | | 0.02% | |
| | | | 0.04% | |
| | | | 0.08% | |
| Benzalkonium Chloride | Alfa Aesar | B20760 | 0.01% | ~0.01% |
| (BZK) | | | 0.02% | |
| | | | 0.04% | |
| | | | 0.08% | |
| LDAO + BZK | not | not | 0.02% + | not |
| | applicable | applicable | 0.02% | applicable |
| Tween 80 | Thermo | 28329 | 0.02% | 0.002% |
| | Fisher | | | |
| | Scientific | | | |
| Tergitol 15-S-9 | EMD | 15S9 | 0.01% | 0.012% |
| | Millipore | | 0.02% | |
| | | | 0.04% | |
| | | | 0.08% | |
| Empigen BB | EMD | 324690 | 0.04% | 0.044% |
| | Millipore | | | |
| Dodecylethyldimethylammonium | Alrich | 44165 | 0.04% | 0.048% |
| bromide (EDMAB) | | | | |
| Didecyldimethylammonium | TCI | D2363 | 0.02% | 0.003% |
| bromide (DDAB) | | | | |
| Zwittergent 3-14 | EMD | 693017 | 0.01% | 0.006% |
| | Millipore | | 0.02% | |
| | | | 0.04% | |
| | | | 0.08% | |
| Brij35 | Thermo | 28316 | 0.02% | 0.011% |
| | Fisher | | 0.04% | |
| | Scientific | | | |
| CHAPSO | EMD | 2202020 | 0.20% | 0.500% |
| | Millipore | | 0.50% | |
| NDSB201 | EMD | 480005 | 0.20% | not |
| | Millipore | | | applicable |
| Tergitol 15-S-40 | EMD | STS0003 | 0.10% | 0.131% |
| | Millipore | | 0.20% | |
| NP-40 alternative | Calbiochem | 492016 | 0.02% | ~0.017% |

In addition to containing a surfactant, the treatment solutions also contained sucrose, Tris, pH 7.5, EDTA, pH8.0, and antifoam SE-15 as described in Table 3. The solutions did not contain a protease.

TABLE 3
Additional Components
Surfactant Treatment Solution

| Component | Vendor | Part No. | Concentration | |
|---|---|---|---|---|
| 2X Surfactant | NA | NA | 2X | |
| from Table 2 | | | | |
| Tris-HCl | Thermo Fisher | | 10 | mM |
| | Scientific | | | |
| EDTA | Thermo Fisher | | 0.75 | mM |
| | Scientific | | | |
| Sucrose | Sigma | | 400 | mM |

| | | | |
|---|---|---|---|
| Anti-foam SE-15 | Sigma | A8582 | 0.002% |

As a negative control, 15 μL of the virus sample was combined and mixed with 15 μL of 10 mM Tris, pH 7.5 and 0.75 mM EDTA, pH8.0. The mixtures were incubated at ambient temperature (approximately 20-25° C.) for 30 minutes. Afterwards, mixtures were either heated at 92° C. for 5 minutes and then cooled to 4° C. for at least 2 minutes or kept at room temperature for at least 7 minutes. 5 μL of treated sample was then combined with 15 μL of RT-qPCR mix as indicated in Table 4.

TABLE 4
RT-qPCR mix
RT-qPCR mix

| Component | volume (μL) |
|---|---|
| TaqPath ™ 1-Step | 5 |
| Multiplex Master | |
| mix (no ROX) | |
| Covid-19 Real-time | 2 |
| PCR Assay Multiplex | |
| water | 8 |

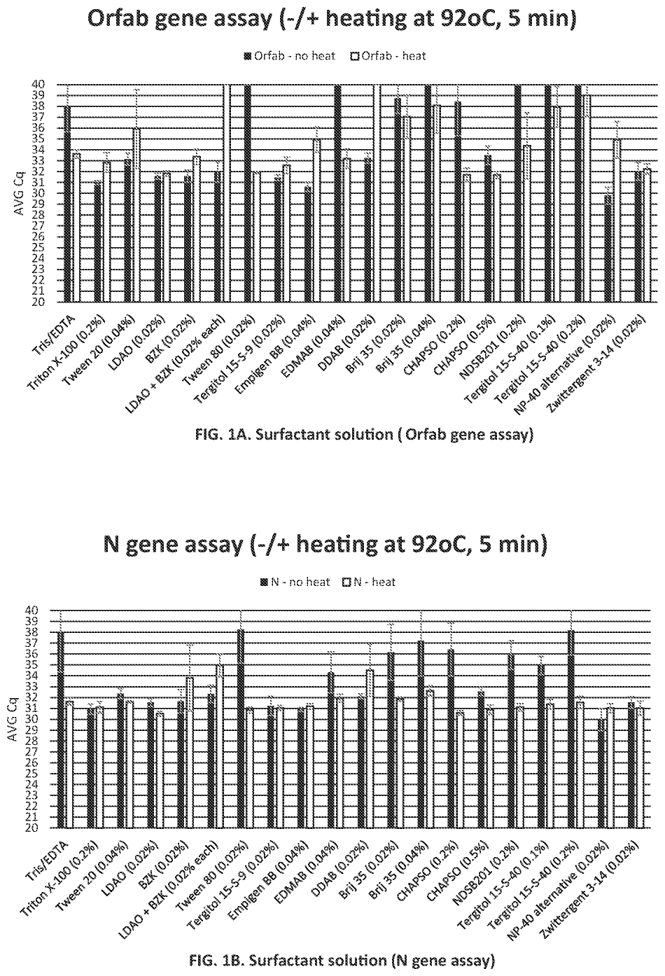

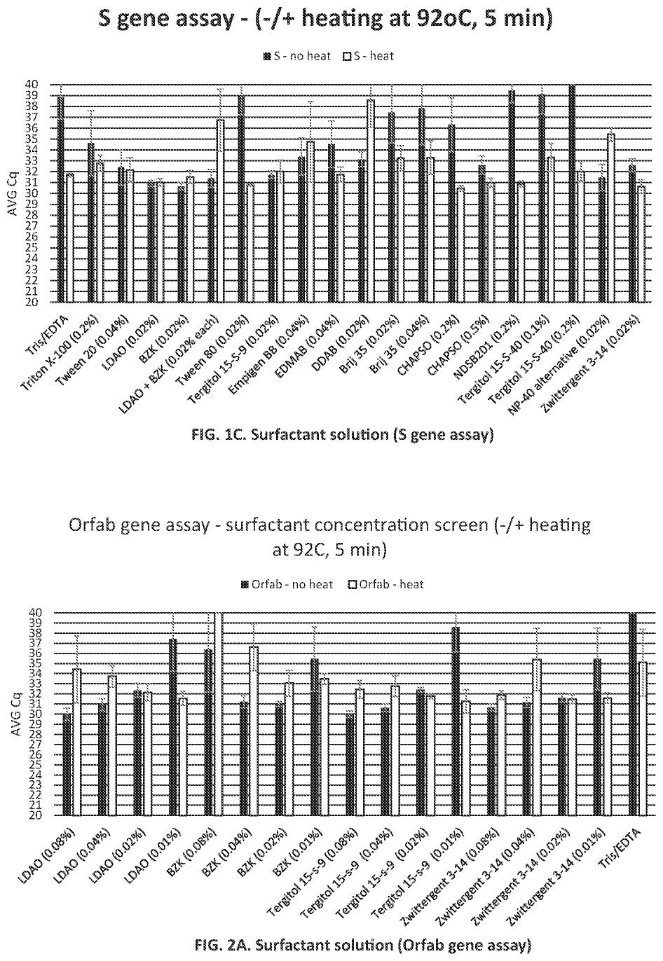

The RT-qPCRs were performed according to the following thermal cycling protocol: 25° C. for 2 minutes; 48° C., 10 minutes followed by 95° C., 10 minutes; and 40 cycles of 95° C. for 3 seconds followed by 60° C. for 30 seconds. The TaqPath™ COVID-19 RT-PCR kit (Thermo Fisher Scientific, Part No. A47814) that was used for these studies contained TaqPath™ 1-Step Multiplex Master mix (no ROX) and Covid-19 Real-time PCR Assay Multiplex. This kit measured 3 genomic RNA targets (N, S, and Orfab genes) in a multiplexed RT-qPCR. The average Cq values of 4 replicate wells obtained for each test condition and genomic RNA assay are shown in FIGS. 1A-1C. It was observed that several surfactants gave Cq values that were the same or similar to those from virus in Tris-EDTA buffer in the absence of a heating step, which indicated that they were ineffective at disrupting the virus particles and making the genome accessible for direct RT-qPCR. Examples include: Tween-80 (0.02%), Brij 35 (0.02 or 0.04%), CHAPSO (0.2%), EDMAB (0.04%), Tergitol 15-S-40 (0.1 and 0.2%), DoTAB (0.45%), and NDSB201 (0.2%). In most cases, heating of the mixtures of virus and surfactant solution resulted in lower Cq values compared to those obtained without heating and performed similarly to mixtures of virus and Tris-EDTA buffer, which indicated that the surfactants were compatible with RT-qPCR at the concentrations that were tested. Examples include: Tween 80, Brij35, CHAPSO, EDMAB and DeTAB.

In contrast, a few surfactants gave average Cq values that were lower than those from virus in Tris-EDTA buffer without heating and similar to those from virus in Tris-EDTA buffer with heating, which showed that they were effective at disrupting the virus particles and making the genome accessible for direct RT-qPCR in the absence of a heating step. Examples include: Triton X-100 (0.2%), Tween 20 (0.04%), LDAO (0.02%); BZK (0.02%), Tergitol 15-S-9, Empigen BB (0.04%), DDAB (0.02%), Zwittergent 3-14 (0.02%) and NP-40 (0.02%). However, in the case of DDAB, heating of the mixture resulted in higher average C_{q} values and therefore was excluded from further testing.

The mixture of LDAO (0.02%) and BZK (0.02%) that was tested was found to be equally effective compared the individual surfactants in the absence of heating. However, this combination was less effective than individual surfactant solutions when the sample mixture was heated to 92° C. for 5 minutes after the initial 30-minute room temperature incubation (FIGS. 1A-1C).

### Example 2: Effect of Surfactant Concentration on Virus Genomic RNA Reverse Transcription and Amplification

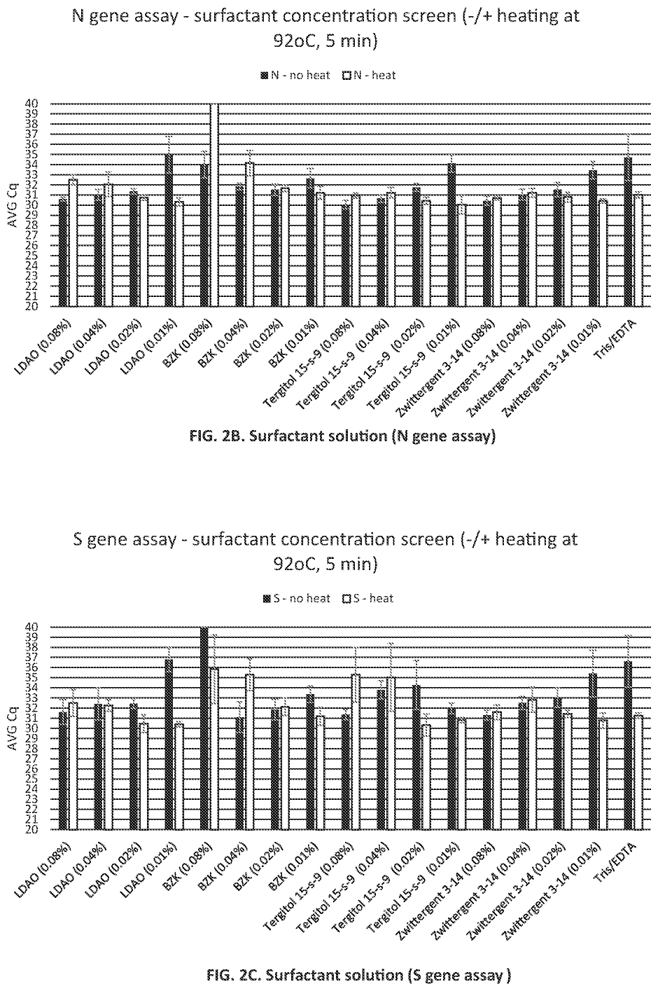

The method described in Example 1 was also used to test the effect of surfactant concentration on virus disruption activity. Four surfactants, LDAO, BZK, Tergitol 15-S-9, and Zwittergent 3-14 were selected for further concentration testing. Solutions were prepared as described above at four surfactant concentrations: 0.01, 0.02, 0.04 and 0.08%. The average Cq values of 4 replicate wells obtained for each test condition are shown in FIGS. 2A-2C.

Results: All four surfactant solutions at the lowest concentration, 0.01%, gave average Cq values that were the same or similar to those from virus in Tris-EDTA buffer, which indicated that they were ineffective at disrupting the virus particles and making the genome accessible for direct RT-qPCR in the absence of heating.

The highest concentration of BZK, 0.08%, gave Cq values that were the same or similar to those from virus in Tris-EDTA buffer, which suggested that higher concentrations of BZK were inhibitory to downstream RT-qPCR. The data also suggested that the effects of higher concentrations of BZK on RT-qPCR were exacerbated by heating at 92° C. for 5 minutes prior to performing the RT-qPCR.

In contrast, concentrations of LDAO, Tergitol 15-S-9, and Zwittergent 3-14 of 0.02-0.08% gave average Cq values that were lower than those from virus in Tris-EDTA buffer without heating and similar to those from virus in Tris-EDTA buffer with heating, which showed that they were effective at disrupting the virus particles and making the genome accessible for direct RT-qPCR in the absence of a heating step. Heating at 92° C. for 5 minutes had minimal effect on the downstream RT-qPCR.

### Example 3: Effect of LDAO Surfactant Solution Treatment Time on Virus Genomic RNA Reverse Transcription and Amplification

The method described in Example 1 was used to test the effect of surfactant treatment solution incubation time on virus disruption activity. A 0.02% LDAO-containing surfactant solution (2×) was used for this testing. The incubation time at room temperature was varied from 0 to 60 minutes. Mixtures of virus in Tris-EDTA solution were used as a negative control. In this study, heating to 92° C. for 5 minutes was not applied to the test samples. After the indicated incubation time at room temperature, the tubes containing the mixtures of virus and surfactant solution were placed on ice. At time zero, TO, mixtures were immediately placed on ice. A control mixture of virus in Tris-EDTA was also incubated at room temperature for 60 minutes, T60, and then placed on ice. Mixtures of virus and LDAO-containing solution were incubated at room temperature for 0, 1, 2, 5, 10, 15, 30, and 60 minutes prior to being transferred to ice. At the end of the 60-minute incubation time, the TO conditions were repeated for both mixtures (with Tris-EDTA or LDAO-containing solutions) on ice immediately prior to setting up the RT-qPCRs.

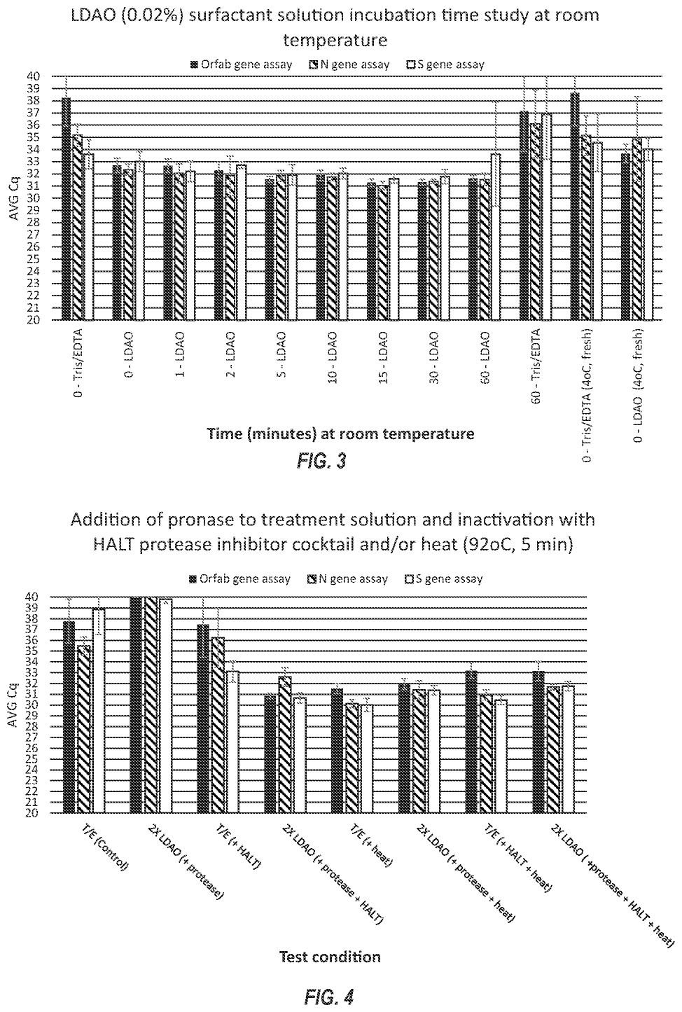

Results: It was observed that for the LDAO-containing solution, the average Cq values of 4 replicates at time zero (TO) were lower than those from virus in Tris-EDTA buffer at 0 or 60 minutes of incubation at room temperature, which indicated that the LDAO-containing treatment solution rapidly disrupted the virus particles (FIG. 3). Average Cq values for the LDAO-containing treatment solution were only slightly lower with longer incubation time at room temperature up to about 15 minutes compared to 0 and 1 minute test conditions. Furthermore, the time zero treatments were repeated after completing the 60-minute incubation period, which confirmed the rapid disruption of the virus particles by the LDAO-containing treatment solution.

### Example 4: Effect of Protease Treatment and Subsequent Inactivation with Protease Inhibitor Cocktail (HALT) or Heating on Virus Genomic RNA Reverse Transcription and Amplification

The method described in Example 1 was also used to test the effect of including a protease in the surfactant treatment solution. In Example 4, RNASE- and DNASE-free pronase (Millipore, Part No. 537088) was added at a concentration of 57 U/mL to an LDAO-containing treatment solution additionally including the components of Table 3. Mixtures of virus sample and Tris-EDTA solution were tested without the addition of protease as controls. All dilution, mixing, and incubation steps were the same as described in Example 1. However, after the room temperature incubation step and prior to RT-qPCR, the mixtures were either: 1) heated at 92° C. for 5 minutes and then cooled to 4° C. for 2 minutes with or without the addition of HALT protease inhibitor cocktail (Thermo Fisher Scientific, Part No. 78430) to a final concentration of 1×; or 2) kept at room temperature with or without the addition of HALT protease inhibitor cocktail for at least 7 minutes prior to RT-qPCR.

Results: It was observed that the addition of a protease to the treatment solution is compatible with the method in Example 1, and notably, with RT-qPCR steps when used in combination with: 1) a heating step at 92° C. for 5 minutes, or, 2) the addition of the HALT protease inhibitor cocktail with or without the heating step (FIG. 4). However, the inclusion of pronase without subsequent inactivation either with heating at 92° C. for 5 minutes or adding HALT protease inhibitor cocktail resulted in no RT-qPCR amplification (Cq=40).

### Example 5: Treatment of SARS-Cov-2 with Surfactant and Protease Solutions Reduces Virus Titer

Multiple treatment solutions and conditions were tested for efficacy of SARS-CoV-2 virus titer reduction in Vero E6 cells. Each condition was tested in a 3-fold dilution series. Infected and uninfected cells were evaluated for cytopathic effects in order to assess the 50% inhibitory concentration (TCID50) of the test conditions. The treatment solution compositions, incubation temperatures and incubation times were tested as described in Table 5.

TABLE 5
TCID50 test conditions and results
TCID50 test conditions and results

| | | | Time | Temp. | log10 |
|---|---|---|---|---|---|
| Condition | Description | Buffer (2X) | (minutes) | (C.) | TCID50/mL |
| 1 | positive control - | 10 mM Tris pH 7.5, | 30 | 65 | 0 |
| | buffer only + heat | 0.75 mM EDTA, pH 8.0 | | | |
| 2 | negative control - | 10 mM Tris pH 7.5, | 60 | 18-25 | 4.67 |
| | buffer only | 0.75 mM EDTA, pH 8.0 | | | |
| 3 | no surfactant, no | 10 mM Tris pH 7.5, | 60 | 18-25 | 4.67 |
| | pronase | 0.75 mM EDTA, pH 8.0, | | | |
| | | 400 mM Sucrose, 0.002% | | | |
| | | anti-foam SE15 | | | |
| 4 | no surfactant, | 10 mM Tris pH 7.5, | 60 | 18-25 | 4.5 |
| | 57 U/mL pronase | 0.75 mM EDTA, pH 8.0, | | | |
| | | 400 mM Sucrose, 0.002% | | | |
| | | anti-foam SE15 | | | |
| 5 | 0.02% LDAO, no | 10 mM Tris pH 7.5, | 60 | 18-25 | 4.5 |
| | pronase | 0.75 mM EDTA, pH 8.0, | | | |
| | | 400 mM Sucrose, 0.002% | | | |
| | | anti-foam SE15 | | | |
| 6 | 0.04% LDAO, no | 10 mM Tris pH 7.5, | 60 | 18-25 | 4.5 |
| | pronase | 0.75 mM EDTA, pH 8.0, | | | |
| | | 400 mM Sucrose, 0.002% | | | |
| | | anti-foam SE15 | | | |
| 7 | 0.02% LDAO, | 10 mM Tris pH 7.5, | 30 | 18-25 | 4.5 |
| | 57 U/mL pronase | 0.75 mM EDTA, pH 8.0, | | | |
| | | 400 mM Sucrose, 0.002% | | | |
| | | anti-foam SE15 | | | |
| 8 | 0.02% LDAO, | 10 mM Tris pH 7.5, | 60 | 18-25 | 4 |
| | 57 U/mL pronase | 0.75 mM EDTA, pH 8.0, | | | |
| | | 400 mM Sucrose, 0.002% | | | |
| | | anti-foam SE15 | | | |
| 9 | 0.02% LDAO, | 10 mM Tris pH 7.5, | 30 | 37 | 3.33 |
| | 57 U/mL pronase | 0.75 mM EDTA, pH 8.0, | | | |
| | | 400 mM Sucrose, 0.002% | | | |
| | | anti-foam SE15 | | | |
| 10 | 0.02% LDAO, | 10 mM Tris pH 7.5, | 60 | 37 | 2.5 |
| | 57 U/mL pronase | 0.75 mM EDTA, pH 8.0, | | | |
| | | 400 mM Sucrose, 0.002% | | | |
| | | anti-foam SE15 | | | |
| 11 | 0.04% LDAO, | 10 mM Tris pH 7.5, | 30 | 18-25 | 3.33 |
| | 57 U/mL pronase | 0.75 mM EDTA, pH 8.0, | | | |
| | | 400 mM Sucrose, 0.002% | | | |
| | | anti-foam SE15 | | | |
| 12 | 0.04% LDAO, | 10 mM Tris pH 7.5, | 60 | 18-25 | 3.33 |
| | 57 U/mL pronase | 0.75 mM EDTA, pH 8.0, | | | |
| | | 400 mM Sucrose, 0.002% | | | |
| | | anti-foam SE15 | | | |
| 13 | 0.04% LDAO, | 10 mM Tris pH 7.5, | 30 | 18-25 | 2.5 |
| | 57 U/mL pronase | 0.75 mM EDTA, pH 8.0, | | | |
| | | 400 mM Sucrose, 0.002% | | | |
| | | anti-foam SE15 | | | |
| 14 | 0.04% LDAO, | 10 mM Tris pH 7.5, | 60 | 18-25 | 2.5 |
| | 57 U/mL pronase | 0.75 mM EDTA, pH 8.0, | | | |
| | | 400 mM Sucrose, 0.002% | | | |
| | | anti-foam SE15 | | | |
| 15 | 0.02% | 10 mM Tris pH 7.5, | 30 | 18-25 | 2.5 |
| | Benzalkonium | 0.75 mM EDTA, pH 8.0, | | | |
| | Chloride, 57 U/mL | 400 mM Sucrose, 0.002% | | | |
| | pronase | anti-foam SE15 | | | |
| 16 | 0.02% | 10 mM Tris pH 7.5, | 60 | 18-25 | 2.5 |
| | Benzalkonium | 0.75 mM EDTA, pH 8.0, | | | |
| | Chloride, 57 U/mL | 400 mM Sucrose, 0.002% | | | |
| | pronase | anti-foam SE15 | | | |
| 17 | 0.02% | 10 mM Tris pH 7.5, | 30 | 37 | 2.5 |
| | Benzalkonium | 0.75 mM EDTA, pH 8.0, | | | |
| | Chloride, 57 U/mL | 400 mM Sucrose, 0.002% | | | |
| | pronase | anti-foam SE15 | | | |
| 18 | 0.02% | 10 mM Tris pH 7.5, | 60 | 37 | 2.5 |
| | Benzalkonium | 0.75 mM EDTA, pH 8.0, | | | |
| | Chloride, 57 U/mL | 400 mM Sucrose, 0.002% | | | |
| | pronase | anti-foam SE15 | | | |

The test protocol involved spiking a human saliva sample with infectious SARS-Cov-2 virus. 10 μL of virus stock solution (titer=6.7) was added to 90 μL of raw human saliva. 100 μL of spiked saliva was then combined with 100 μL of test solution to form a mixture as indicated in Table 5. The test mixtures were incubated according to the times and temperatures for each test condition as indicated in Table 5. After completing the incubations, 2 μL of 100×HALT Protease Inhibitor Cocktail (Thermo Fisher Scientific, Part No. 78430) and 2 μL of 0.5M EDTA, pH 8.0 solution (provided with the protease cocktail) was added to each sample mixture and vortexed for 5 seconds on the highest setting. Three-fold serial dilutions of each sample mixture were prepared in DMEM and then 100 μL of test sample was plated on Vero E6 cells that were cultured to approximately 80% confluency and then incubated at 37±2° C., 5%±2% CO₂, and ≥70% relative humidity for up to 5 days. Each condition was tested in quadruplicate culture wells. At multiple time intervals, the cultures were assessed for cytopathic effects. TCID50 values were calculated using the Reed-Muench Method (Reed, L. J.; Muench, H. “A simple method of estimating fifty percent endpoints” The American Journal of Hygiene 27:493-497, 1938), the disclosure of which is incorporated by reference herein.

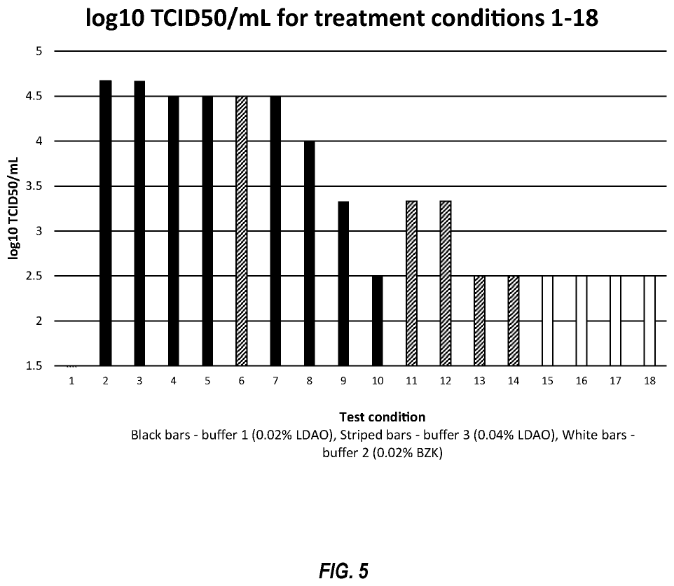

It was observed that SARS-CoV-2 virus that was spiked into saliva and diluted 1:1 in Tris-EDTA buffer was effectively inactivated when the sample was heated to 65° C. for 30 minutes prior to cell culture testing (FIG. 5, condition 1) as indicated by having a virus titer below the limit of detection of the test (<1.5 log 10 TCID50). In contrast, virus-spiked saliva sample in Tris-EDTA buffer without heat treatment at 65° C. (condition 2) maintained a high virus titer of about 4.67 log 10 TCID50. Similarly, test conditions 3-6 maintained high virus titers. For these conditions, test samples were incubated at room temperature for 60 minutes in treatment solutions containing Tris-EDTA buffer, sucrose, and anti-foam SE15 but did not contain both LDAO and pronase. Conditions 7-10 showed a reduction in virus titer with prolonged treatment of the sample with buffer containing 0.02% LDAO and 57 U/mL pronase and treatment at elevated temperature. Conditions 11-14 with 0.04% LDAO and 57 U/mL pronase showed a greater reduction in virus titer compared to the previous set of conditions, especially when treatment was performed at 37° C. Finally, conditions 15-18 with 0.02% benzalkonium chloride and 57 U/mL pronase showed a similar, marked reduction in virus titer for all test conditions, even at ambient temperature for 30 minutes.

Note that the combined use of a surfactant and a protease provided a compounded effect. For example, condition 8 (0.02% LDAO, 57 U/mL pronase) resulted in a lower virus titer than condition 4 (no surfactant, 57 U/mL pronase) or condition 5 (0.02% LDAO, no pronase) given the same temperature and heating time conditions. Similarly, condition 12 (0.04% LDAO, 57 U/mL pronase) resulted in a lower virus titer than condition 4 (no surfactant, 57 U/mL pronase) or condition 6 (0.04% LDAO, no pronase) given the same temperature and heating time conditions.

### Example 6: Sensitive Detection of SARS-CoV-2 Genomic RNA in Saliva Treated with Solutions Containing Surfactant and Protease Mixture

Based on the methods described in Examples 1 and 5, a new method was devised for detecting SARS-CoV-2 RNA in raw saliva samples that contain virus particles without the need for a nucleic acid purification step. This method involved a first step of diluting a raw saliva sample in a treatment solution that contains a surfactant, protease, disaccharide, buffer, chelating agent, and anti-foaming agent whereby a raw saliva sample and a treatment solution were combined in equal parts (1:1) to form a mixture. In this method, the raw saliva sample was first spiked with gamma-irradiated SARS-CoV-2 virus (BEI Resources, Part No. NR-52287). An exemplary treatment solution included 0.02% LDAO, 57 U/mL pronase, 10 mM Tris pH 7.5, 0.75 mM EDTA pH 8.0, 400 mM sucrose, and 0.002% antifoam SE-15. In a second step, the mixtures were heated sequentially at 40° C. for 5 minutes, 92° C. for 5 minutes, and 4° C. for at least 2 minutes. Alternatively, the mixtures were kept at room temperature (18-25° C.) for 30 minutes and subsequently heated sequentially at 92° C. for 5 minutes and 4° C. for at least 2 minutes.

It was theorized that the combination of treatment solution components and heat incubation steps facilitated the release and stabilization of RNA from virus particles and reduced matrix interference in downstream detection assays such as RT-qPCRs. In order to illustrate this concept, gamma-irradiated SARS-CoV-2 virus particles were spiked into three raw saliva samples at a concentration of 8333 copies/mL of sample, processed with the new treatment method and formulation, and tested in RT-qPCRs. The final RT-PCRs contained the equivalent of 50 virus copies in each RT-PCRs. Several other conditions were tested as listed in Table 6. For example, in some conditions, heat and/or dilution of the samples was omitted. Additionally, in some conditions, protease and/or surfactant were omitted.

TABLE 6
RT-qPCR test conditions

| Condition | Description | Buffer (2X) | Heat treatment |
|---|---|---|---|
| 1 | Neat (undiluted) saliva, no heat | None | None |
| | treatment | | |
| 2 | saliva + TE, diluted 1:1, no heat | 10 mM Tris, 0.1 mM | None |
| | treatment | EDTA, pH 8.0 | |
| 3 | saliva + buffer (no pronase), | 10 mM Tris pH 7.5, | None |
| | diluted 1:1, no heat treatment | 0.75 mM EDTA, pH 8.0, | |
| | | 400 mM Sucrose, 0.02% | |
| | | LDAO, 0.002% anti- | |
| | | foam SE-15 | |
| 4 | Neat (undiluted) saliva, with | None | 40° C. for 5 minutes, 95° C. |
| | heat treatment | | for 5 minutes, 4° C. for 2 |
| | | | minutes |
| 5 | saliva + TE, diluted 1:1, with | 10 mM Tris, 0.1 mM | 40° C. for 5 minutes, 95° C. |
| | heat treatment | EDTA, pH 8.0 | for 5 minutes, 4° C. for 2 |
| | | | minutes |
| 6 | saliva + buffer (no pronase), | 10 mM Tris pH 7.5, | 40° C. for 5 minutes, 95° C. |
| | diluted 1:1, no heat treatment | 0.75 mM EDTA, pH 8.0, | for 5 minutes, 4° C. for 2 |
| | | 400 mM Sucrose, 0.02% | minutes |
| | | LDAO, 0.002% anti- | |
| | | foam SE-15 | |
| 7 | saliva + buffer (no surfactant), | 10 mM Tris pH 7.5, | 40° C. for 5 minutes, 95° C. |
| | diluted 1:1, with heat treatment | 0.75 mM EDTA, pH 8.0, | for 5 minutes, 4° C. for 2 |
| | | 400 mM Sucrose, 57 U/mL | minutes |
| | | pronase, 0.002% anti- | |
| | | foam SE-15 | |
| 8 | saliva + buffer (with surfactant | 10 mM Tris pH 7.5, | 40° C. for 5 minutes, 95° C. |
| | and pronase), diluted 1:1, with | 0.75 mM EDTA, pH 8.0, | for 5 minutes, 4° C. for 2 |
| | heat treatment | 400 mM Sucrose, 57 U/mL | minutes |
| | | pronase, 0.002% anti- | |
| | | foam SE-15 | |

The TaqPath™ COVID-19 Combo kit that measures three genomic RNA targets (N, S, and Orflab genes) in a multiplexed RT-qPCR was used for these studies as described above. The RT-qPCRs included 5 μL of TaqPath™ 1-step Multiplex Master Mix (no ROX), 1 μL of Covid-19 Real-time PCR Assay Multiplex assay, 2 μL MS2 phage control provided with the kit, and 12 μL of treated sample and performed according to the following thermal cycling protocol: 25° C. for 2 minutes; 53° C., 10 minutes followed by 95° C., 10 minutes; and 40 cycles of 95° C. for 3 seconds followed by 60° C. for 30 seconds. RT-PCRs were performed in triplicate for each sample and test condition.

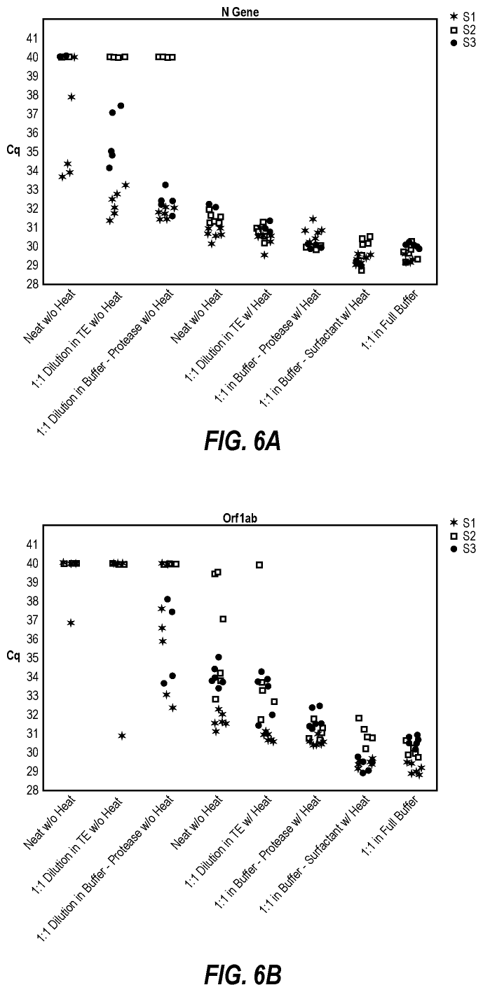

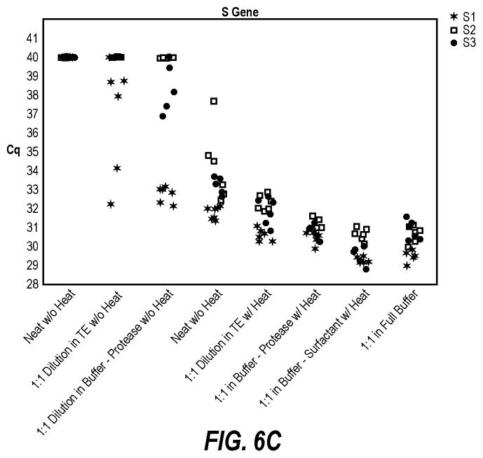

Results: It was observed that the exemplary treatment solution compositions described above provided robust detection of all three SARS-CoV-2 RNA targets and performed better than virus-spiked samples that were either diluted in buffer without surfactant, protease, and sucrose or not diluted and/or heat treated (FIG. 6). It was further observed that treatment solutions that contained surfactant or pronase or a combination thereof performed better than samples that were undiluted or diluted in buffer only. Specifically, the treatment solutions containing surfactant and/or pronase in combination with heating steps produced lower Cq values for the three SARS-CoV-2 assays and all three test samples. Poor performing conditions resulted in the failure to detect some or all samples or assay replicates (Cq=40). Moreover, the inclusion of a surfactant and/or protease in combination with heating steps resulted in improved technical replicate precision compared to other test conditions. The experiment also showed that the presence of a surfactant produced lower Cq values even in the absence of heat treatment. Additionally, it was observed that heating saliva samples as described above could improve assay performance and result in lower Cq values compared to samples that were not subjected to heat treatment. Treatment solution containing 1 mM sodium citrate pH 6.5 in place of Tris pH 7.5 performed similarly (data not shown).

### Example 7: Sensitive Detection of SARS-CoV-2 Genomic RNA in Saliva Treated with Solutions Containing Surfactant and Protease

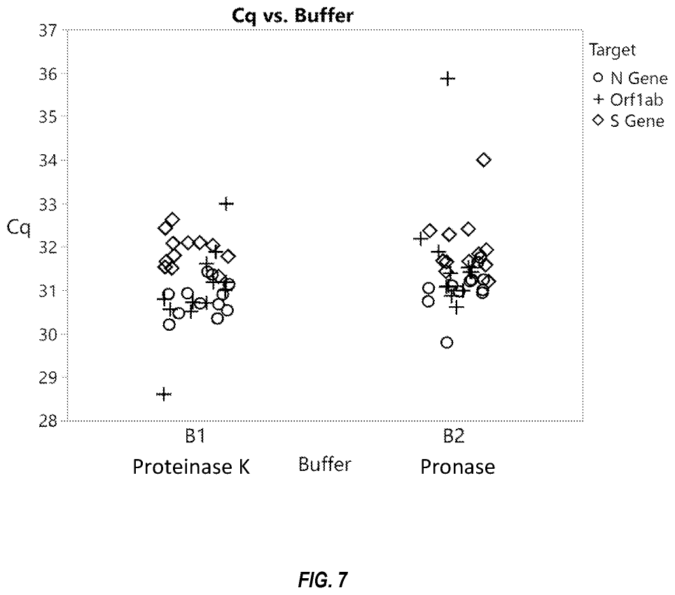

Proteinase K and pronase were tested in additional exemplary treatment solutions with compositions that included 0.02% LDAO, 10 mM Tris pH 8.0, 0.1 mM EDTA pH 8.0, 250 mM sucrose, and either 0.8 mg/mL Proteinase K (Thermo Fisher Scientific, Part No. A42363) 50 U/mL pronase. In this method, a raw saliva sample was first spiked with gamma-irradiated SARS-CoV-2 virus (BEI Resources, Part No. NR-52287) at a concentration of 5000 copies per mL. In a first step, the sample was mixed 1:1 with the exemplary composition containing either Proteinase K or pronase. In a second step, the mixtures were heated sequentially at 62° C. for 5 minutes, 92° C. for 5 minutes, and 4° C. for at least 2 minutes. The TaqPath™ COVID-19 Combo kit that measured three genomic RNA targets (N, S, and Orflab genes) in a multiplexed RT-qPCR was used for these studies as described above. The experiment showed that treatment solution containing Proteinase K produced C_{q} values that were similar to the treatment solution containing pronase (FIG. 7).

### Example 8: Optimization of a Treatment Solution Composition for Sensitive Detection of SARS-CoV-2 Genomic RNA in Saliva

A series of conditions were tested in order to discover an optimal composition for a treatment solution that enables sensitive detection of SARS-CoV-2 genomic RNA in saliva. Parameters that were tested included the concentrations of pronase, LDAO, sucrose, and EDTA, and the pH of Tris-HCl. The testing was performed according to the method in Example 6. Results were analyzed with JMP software, version 14 (SAS Institute) and characterized for optimal desirability across the test parameters. It was observed that a solution formulation containing 57 U/mL pronase, 400 mM sucrose, 0.02% LDAO, 0.75 mM EDTA, and Tris-HCl at a pH Of 7.5 produced optimal results for test parameters of Cq value and delta Rn (dRn) signal value.

### Example 9: Sensitive Detection of SARS-CoV-2 Genomic RNA in Saliva Treated with Solutions Containing Surfactant and Protease

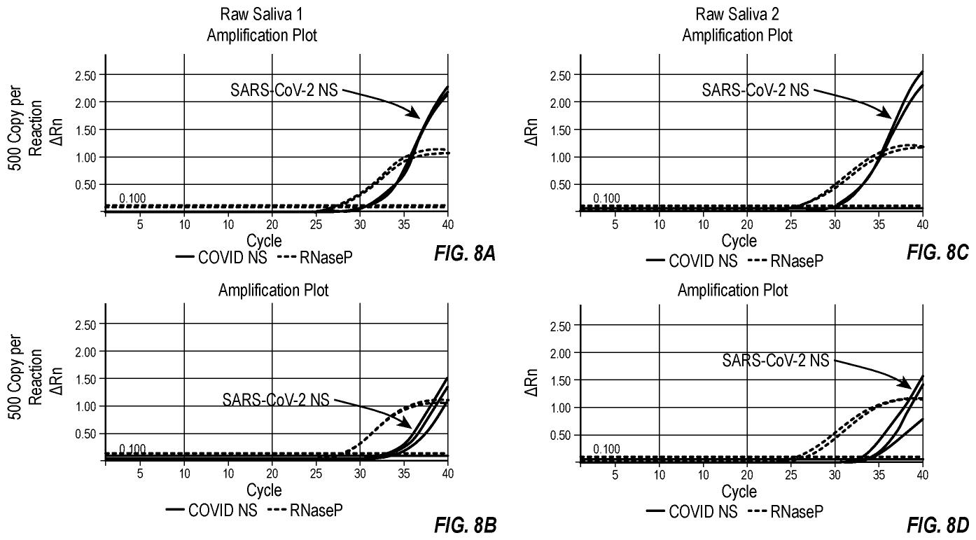

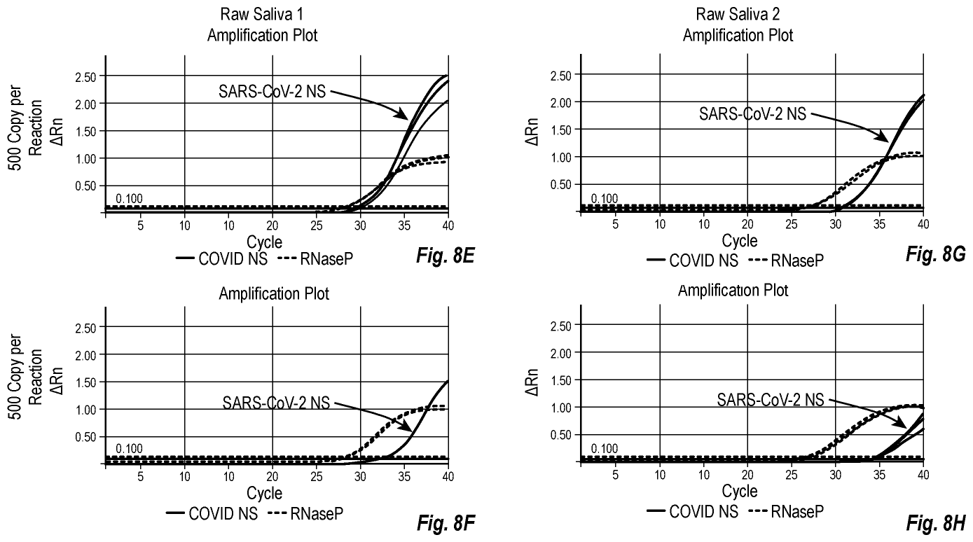

The method described in Example 6 was used to test other exemplary formulations that contained either 0.04% LDAO or 0.02% Benzalkonium Chloride in place of 0.02% LDAO. The formulations also included 57 U/mL pronase, 10 mM Tris pH 7.5, 0.75 mM EDTA pH 8.0, 400 mM sucrose, and 0.002% antifoam SE-15. In this experiment, two raw saliva samples were spiked with gamma-irradiated SARS-CoV-2 virus (BEI Resources Part No. NR-52287) at a concentration of 71430 and 7143 GCE/mL, respectively. In addition, the duplex TaqCheck™ SARS-CoV-2 Fast PCR assay (Thermo Fisher Scientific, Part No. A47693) was used to measure 2 genomic RNA targets (N and S genes) with VIC probes and human RNase P with a FAM probe to measure human nucleic acids from the sample. The RT-qPCRs included 5 μL of TaqPath 1-Step RT-qPCR Master Mix CG with ROX (Thermo Fisher Scientific, Part No. A15300), 1 μL of TaqCheck™ SARS-CoV-2 Fast PCR assay and 14 μL of treated sample and performed according the following thermal cycling protocol: 25° C. for 2 minutes; 53° C., 2 minutes followed by 95° C., 10 minutes; and 40 cycles of 95° C. for 3 seconds followed by 60° C. for 30 seconds. It was observed that the two exemplary treatment solution compositions described above provided robust detection in the two samples of both the SARS-CoV-2 RNA targets with the VIC probe and endogenous human RNase P target nucleic acid (DNA and RNA) that was present in the saliva samples with the FAM probe (FIGS. 8A through 8G).

### Example 10: SARS-CoV-2 Genomic RNA is Stable in Mixtures of Saliva and Treatment Solutions Containing Surfactant and Protease

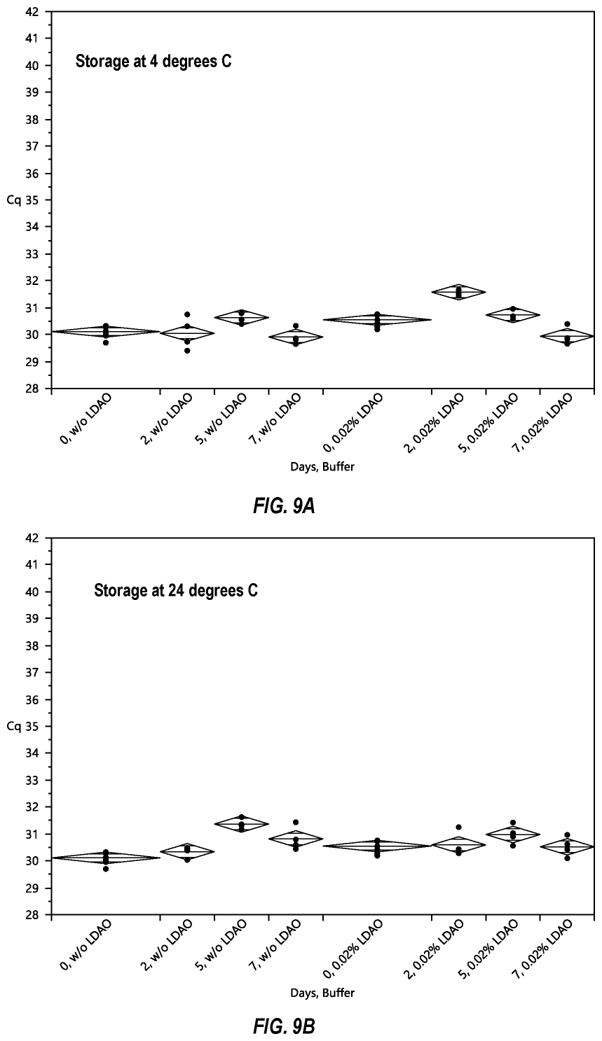

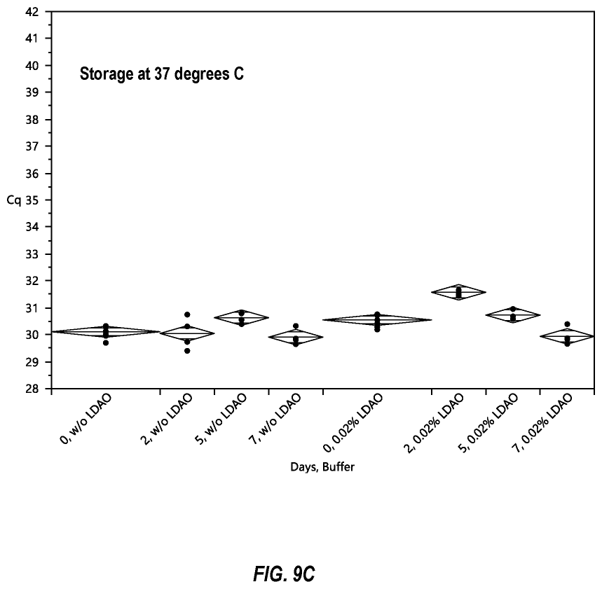

The stability of SARS-CoV-2 genomic RNA in saliva was investigated over a period of 7 days. A pooled saliva sample was spiked with 5000 GCE/mL of gamma-irradiated SARS-CoV-2 and distributed in aliquots that were stored at 4° C., 24° C., or 37° C. for up to 7 days. At days 0, 2, 5, and 7, aliquots from each storage temperature were according to the method in Example 9. It was observed that the SARS-CoV-2 was stable at 4° C. and 24° C. for 7 days; however, detection of viral RNA was reduced after 2 days at 37° C. (FIGS. 9A through 9C). Detection of human RNase P nucleic acid was also reduced after 2 days at all three temperatures (data not shown).

### Example 11: Sensitive Detection of SARS-CoV-2 Genomic RNA in Nasal and Oral Swab Suspensions Treated with Solutions Containing Surfactant and Protease

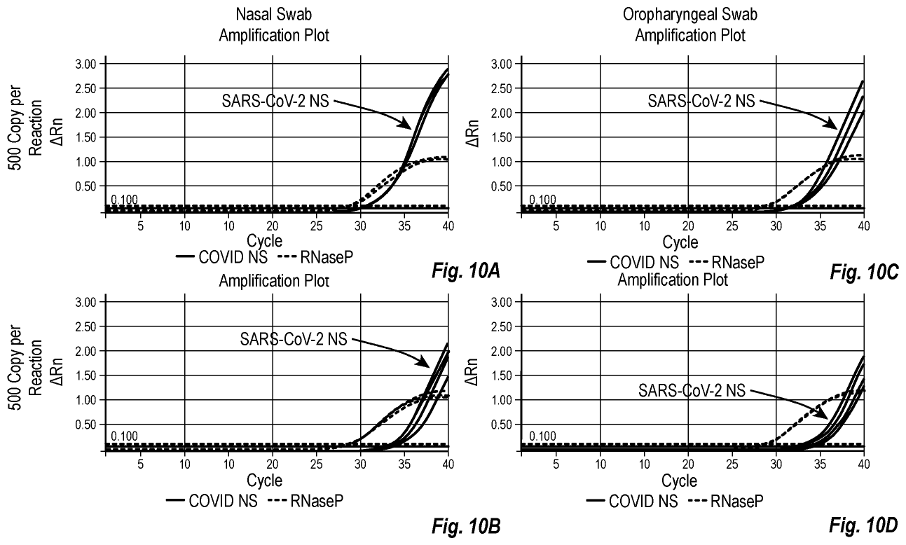

Based on the methods described in Example 6, a new method was devised for detecting SARS-CoV-2 RNA in dry nasal or oral swab samples that contain virus particles without the need for a nucleic acid purification step. This method involved a first step of resuspending the dry swab sample in 200 μL of a treatment solution that contains a surfactant, protease, disaccharide, buffer, chelating agent, and anti-foaming agent to form a mixture. Resuspended dry swab solutions were then spiked to a level of 7125 or 1425 GCE/mL of gamma-irradiated SARS-CoV-2 virus (BEI Resources, Part No. NR-52287). In a second step, an aliquot of the mixture of sample and solution was heated sequentially at 40° C. for 5 minutes, 92° C. for 5 minutes, and 4° C. for at least 2 minutes. The resulting solution was tested for the detection of SARS-Cov-2 N and S gene RNA and human RNase P DNA and RNA with the TaqCheck™ SARS-CoV-2 Fast PCR Assay (Thermo Fisher Scientific, Part No. A47693) as described in Example 9, except the final reaction volume was scaled down to 10 μL. Detection of both Covid-19 and human RNase P targets was observed for the exemplary nasal and oral swab samples (FIGS. 10A through 10D).

### Example 12: Improved Performance for Detection of SARS-CoV-2 Genomic RNA in Saliva Samples Treated with Solutions Containing a Protease

The method in Example 9 was compared to extraction-free procedures described in Ranoa et al (Saliva-Based Molecular Testing for SARS-CoV-2 that Bypasses RNA Extraction. bioRxiv 2020.06.18.159434) and Vogels et al (SalivaDirect: A simplified and flexible platform to enhance SARS-CoV-2 testing capacity. medRxiv 2020.08.03.20167791) using the same saliva samples. The treatment solution formulation described in Example 6 and associated heating conditions were used for this comparison. In this experiment, 24 individual saliva samples were spiked with 1000 GCE/mL of gamma-irradiated SARS-CoV-2 virus. Each sample was then processed through the three different extraction-free procedures. In all cases, the duplex TaqCheck™ SARS-CoV-2 Fast PCR assay was used to measure SARS-CoV-2 RNA targets in accordance with the manufacturer's instructions. All RT-qPCR compositions for the three tested procedures consisted of 5 μL of TaqPath 1-Step RT-qPCR Master Mix CG with ROX and 1 μL of TaqCheck™ SARS-CoV-2 Fast PCR Assay (Thermo Fisher Scientific, Part No. A47693); however, the volume of sample and water varied between methods. For the treatment solution and method described herein, 14 μL of sample was used in the RT-PCR. For the method described in Ranoa et al, 10 μL of sample and 4 μL of water were used in the RT-PCR. For the method described in Vogels et al, 5 μL of sample and 9 μL of water were used in the RT-PCR.

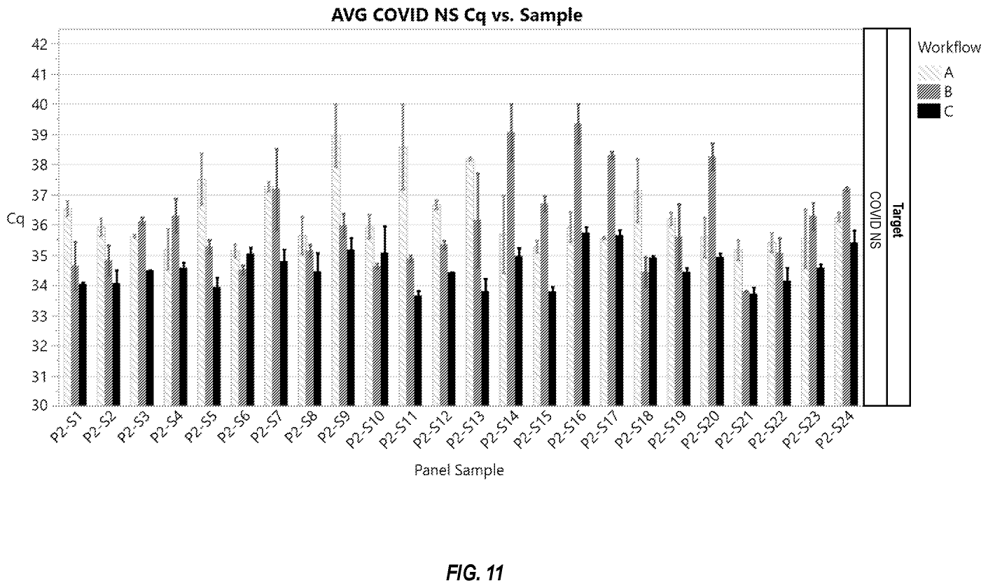

As shown in FIG. 11, the treatment solution and method described herein (Workflow C) resulted in lower average Cq values of 2 replicates for the majority of samples. In addition, the uniformity of Cq values were superior for the test method (Workflow C) as compared to the methods from Ranoa et al (Workflow A) and Vogels et al (Workflow B). Moreover, 100% of Cq values from the test method were below 37 while the methods from Ranoa et al and Vogels et al resulted in Cq values below 37 for 79% and 75% of the sample replicates, respectively.

### Example 13: Improved Detection of Virus Genomic RNA in SARS-CoV-2 Positive Saliva Samples

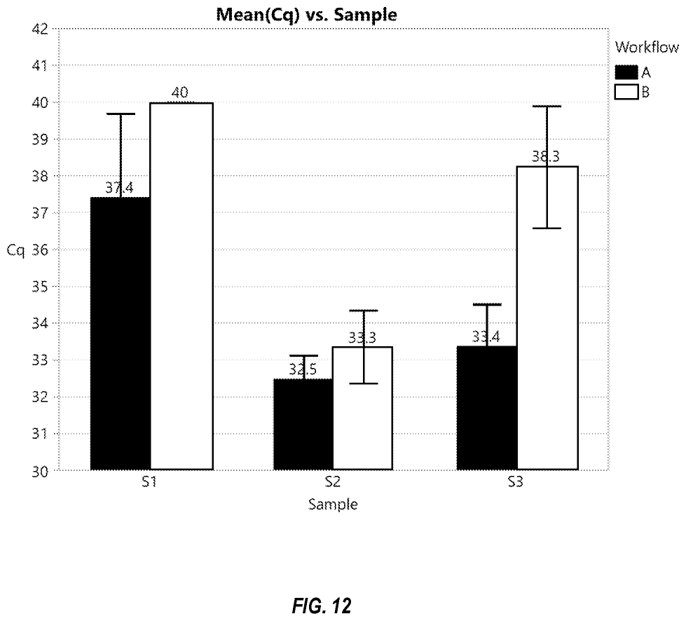

Three saliva samples that were confirmed to be positive for SARS-CoV-2 using standard nucleic acid extraction methods (data not shown), were tested with either the novel method described Example 9 or the method described in Vogels et al (SalivaDirect: A simplified and flexible platform to enhance SARS-CoV-2 testing capacity. medRxiv. doi: https://doi.org/10.1101/2020.08.03.20167791) (at the time of this filing, most recent version posted Sep. 28, 2020). For the novel method described herein, it was observed that all three samples produced Cq values that on average were below 40 (FIG. 12, Workflow A). A Cq=40 indicated that amplification of target nucleic acid was not detected. In contrast, only two of the three samples produced detectable amplification of target nucleic acid with the Vogels et al. method (Workflow B). Moreover, for sample 3 (S3), the disclosed method resulted in a substantially lower average Cq value for 3 replicate wells of 33.4 than the Vogels et al. method which resulted in an average Cq value of 38.3.

### Example 14: Detection of SARS-CoV-2 Virus Genomic RNA in Saliva Samples Treated with Solutions Containing a Protease in a Single Vessel

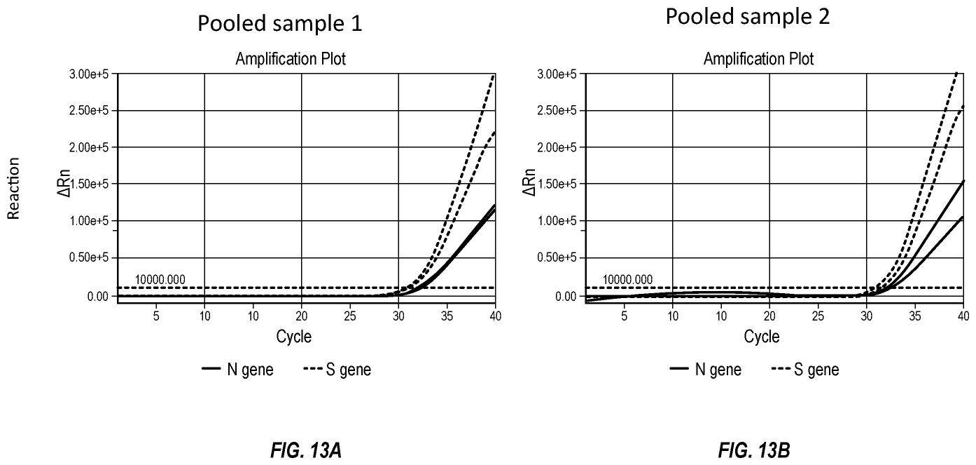

A method was developed based on Example 6 whereby the steps of: 1) combining a saliva sample and the treatment solution, 2) heating the mixture at 40° C. for 5 minutes, 92° C. for 5 minutes, and 4° C. for at least 5 minutes, 3) adding an RT-PCR master mix and PCR assay mixture of PCR primers and TaqMan probes, and 4) performing the RT-PCR thermal cycling steps were performed in a single reaction vessel. In this example, two saliva samples were spiked with 2000 GCE/mL of BEI gamma-irradiated SARS-CoV-2 virus. 15 μL of each spiked saliva sample was mixed with 15 μL of treatment solution in a 96 well PCR plate. The plate was unsealed and 12.5 μL of TaqPath 1-Step Multiplex Master Mix (no ROX), 2.5 μL of TaqPath Covid-19 Real-time PCR Assay Multiplex, and 5 μL of water were added to each well. The reaction plate was sealed with an optical seal, vortexed for 30 seconds and then centrifuged to collect the liquid at the bottom of the wells. The RT-PCRs were performed according to the following thermal cycling protocol: 25° C. for 2 minutes; 53° C., 10 minutes followed by 95° C., 10 minutes; and 40 cycles of 95° C. for 3 seconds followed by 60° C. for 30 seconds. RT-PCRs were performed in triplicate for each sample and test condition. It was observed that the all-in-one-well workflow produced robust detection of 30 GCE/reaction for the N and S genes for both saliva samples (FIGS. 13A and 13B).

### Example Embodiments

Provided below is a non-exhaustive list of numbered items reciting certain preferred embodiments:
1. A composition formulated for mixing with a crude biological sample to enable subsequent analysis of a target nucleic acid, the composition comprising:
   a surfactant;
   a protease component;
   a chelating agent; and
   a buffering salt.
2. The composition of item 1, further comprising a saccharide.
3. The composition of item 2, wherein the saccharide is a disaccharide.
4. The composition of item 3, wherein the disaccharide comprises sucrose, trehalose, or both.
5. The composition of any one of items 1-4, wherein the saccharide is included at a concentration of about 200 mM to about 600 mM.
6 The composition of any one of items 1-5, wherein the surfactant comprises a nonionic detergent.
7. The composition of item 6, wherein the nonionic detergent comprises one or more of nonyl phenoxypolyethoxylethanol (NP-40), TERGITOL 15-S-9, TRITON X-100, or TWEEN 20.
8. The composition of any one of items 1-7, wherein the surfactant comprises a cationic detergent.
9. The composition of item 8, wherein the cationic detergent comprises one or more of benzalkonium chloride (BZK) or didodecyldimethylammonium bromide (DDAB).
10. The composition of any one of items 1-9, wherein the surfactant comprises a zwitterionic detergent.
11. The composition of item 10, wherein the zwitterionic detergent comprises one or more of lauryldimethylamine oxide (LDAO), EMPIGEN BB, or ZWITTERGENT 3-14.
12. The composition of any one of items 1-11, wherein the protease component comprises proteinase K.
13. The composition of any one of items 1-12, wherein the protease component comprises a mixture of proteases.
14. The composition of item 13, wherein the mixture of proteases comprises a mixture of proteases isolated from a Streptomyces culture.
15. The composition of item 14, wherein the protease component comprises pronase.
16. The composition of any one of items 1-15, wherein the buffering salt comprises a sodium salt, such as sodium citrate.
17. The composition of item 16, wherein the sodium salt comprises sodium citrate.
18. The composition of any one of items 1-17, wherein the buffering salt comprises a chloride salt.
19. The composition of item 18, wherein the chloride salt comprises Tris-HCl.
20. The composition of any one of items 1-19, wherein the chelating agent comprises ethylenediaminetetraacetic acid (EDTA) or a conjugate base or salt thereof.
21. The composition of any one of items 1-20, wherein the composition has a total salt concentration less than about 50 mM.
22. The composition of item 21, wherein the composition has a total salt concentration of about 2 mM to about 15 mM.
23. The composition of any one of items 1-22, wherein the composition is formulated for mixing with the crude biological sample at a ratio of about 0.5:1 to about 2:1, or at a ratio of about 1:1, with component amounts of the composition being scaled accordingly for other mixture ratios.
24. The composition of any one of items 1-23, wherein the surfactant is included at a concentration of about 0.01% to about 0.10% w/v, or more preferably about 0.02% to about 0.08% w/v.
25. The composition of any one of claims 1-24, wherein the surfactant is included at a concentration within 0.5× and 15× of the surfactant's critical micelle concentration (CMC).
26. The composition of any one of items 1-25, wherein the protease component is included at a concentration of about 20 U/ml to about 100 U/ml, or more preferably about 35 U/ml to about 85 U/ml, or more preferably about 50 U/ml to about 70 U/ml.
27. The composition of any one of items 1-26, wherein the chelating agent is included at a concentration of about 0.3 mM to about 1.2 mM, or more preferably about 0.5 mM to about 1.0 mM.
28. The composition of any one of items 1-26, wherein the composition has a pH of about 7 or greater.
29. The composition of item 28, wherein the composition has a pH of about 7.2 to about 8.
30. The composition of any one of items 1-29, wherein the composition is formulated for mixing with a crude biological sample comprising one or more of a saliva sample, buccal sample, nasal sample, nasal pharyngeal sample, blood sample, urine sample, or semen sample.
31. The composition of any one of items 1-30, wherein the composition is formulated for subsequent analysis via polymerase chain reaction (PCR).
32. The composition of item 31, wherein the composition is formulated for subsequent analysis via reverse transcription PCR (RT-PCR).
33. The composition of any one of items 1-32, wherein the composition is formulated to enable subsequent analysis of RNA within the crude biological sample.
34. The composition of item 33, wherein the composition is formulated to enable subsequent analysis of viral RNA within the crude biological sample.
35. The composition of item 34, wherein the viral RNA comprises SARS-CoV-2 RNA.
36. The composition of any one of items 1-35, wherein the composition is formulated for mixing with a crude biological sample comprising a mixture of animal cells and/or macromolecules and at least one virus and/or microorganism.
37. The composition of any one of items 1-36, wherein the composition is formulated for mixing with a sample swab such that the crude biological sample is a resuspension of material disposed on the sample swab.
38. The composition of any one of items 1-37, wherein the composition is formulated such that when mixed with the crude biological sample, the resulting solution mixture remains stable at room temperature for at least 96 hours.
39. The composition of any one of items 1-38, wherein the composition is formulated to provide at least two of:
   (i) stabilization of the crude biological sample when mixed;
   (ii) inactivation of at least one virus and/or microorganism within the crude biological sample;
   (iii) lysis of animal cells and/or the at least one virus and/or microorganism within the crude biological sample;
   (iv) reduction in viscosity of the crude biological sample; and
   (v) improving accessibility of viral and/or other microorganism nucleic acids within the crude biological sample.
40. The composition of item 39, wherein the composition is formulated to provide at least three of functions (i)-(v).
41. The composition of any one of items 1-40, the composition being formulated to preserve integrity of nucleic acids within the crude biological sample without extraction or purification of the nucleic acids.
42. The composition of item 41, the composition being formulated to preserve integrity of RNA within the crude biological sample without extraction or purification of the RNA.
43. The composition of any one of items 1-42, the composition being formulated to inactivate at least one virus and/or other microorganism by way of the surfactant, protease component, or both.
44. The composition of any one of items 1-43, wherein the composition is formulated to provide increased access to the target nucleic acid when mixed with the crude biological sample as compared to a mixture of the crude biological sample with water and/or TE buffer.
45. The composition of item 44, wherein the composition is formulated to provide increased access to the target nucleic acid by one or more of disrupting viral envelopes, disrupting cell membranes, or disrupting proteins within the crude biological sample.
46. The composition of any one of items 1-45, further comprising an antifoam agent.
47. The composition of item 46, wherein the antifoam agent is included at a concentration of about 0.001% to about 0.008% w/v, or more preferably about 0.0015% to about 0.004% w/v.
48. The composition of item 46 or item 47, wherein the antifoam agent comprises silicon and nonionic emulsifiers.
49. The composition of item 48, wherein the antifoam agent comprises SE-15.
50. A solution mixture comprising the composition as in any one of items 1-49, and the crude biological sample.
51. The solution mixture of item 50, wherein the crude biological sample includes one or more of a saliva sample, buccal sample, nasal sample, nasal pharyngeal sample, blood sample, urine sample, or semen sample.
52. The solution mixture of item 50, wherein the solution mixture is a resuspension of a dry swab.
53. The solution mixture of item 51, wherein the resuspension has a small resuspension volume of about 0.1 ml to about 1 ml.
54. The solution mixture of any one of items 50-53, wherein the target nucleic acid within the crude biological sample is a viral nucleic acid.
55. The solution mixture of any one of items 50-54, wherein the target nucleic acid is a SARS-CoV-2 nucleic acid.
56. The solution mixture of any one of items 50-55, wherein the solution mixture is stable at room temperature for at least 96 hours.
57. The solution mixture of any one of items 50-56, wherein the crude biological sample makes up about 10% to 60% of the volume of the solution mixture.
58. The solution mixture of any one of items 50-57, wherein the composition and the crude biological sample are mixed at a ratio of about 0.5:1 to about 4:1, or at a ratio of about 1:1 to about 2:1, with component amounts of the composition being scaled accordingly for other mixture ratios.
59. A method for processing a biological sample containing or suspected of containing a target nucleic acid, the method comprising:
(a) contacting the biological sample with a treatment composition comprising a protease component to form a mixture;
(b) inactivating the protease component in the mixture of (a); and
(c) performing an analysis of the target nucleic acid.
60. The method of item 59, wherein the treatment composition is the composition of any one of items 1-49.
61. The method of item 59 or item 60, wherein step (c) comprises amplifying a first target nucleic acid in a biological sample.
62. The method of item 61, wherein step (c) comprises amplifying multiple target nucleic acids in a biological sample.
63. The method of item 62, wherein a second target nucleic acid is derived from an organism from which the biological sample is obtained.
64. The method of item 63, wherein the second target nucleic acid is an RNase P nucleic acid.
65. The method of any one of items 61-64, wherein the amplification reaction is PCR.
66. The method of item 65, wherein the PCR is RT-PCR.
67. The method of item 66, wherein the RT-PCR is a one-step RT-PCR.
68. The method of any one of items 65-67, wherein the PCR is a quantitative PCR (qPCR).
69. The method of any one of items 61-68, further comprising detecting the amplified target nucleic acid during or after the amplification step.
70. The method of any one of items 61-69, further comprising determining the amount of the target nucleic acid present in the crude biological sample.
71. The method of item 70, wherein recovery of the target nucleic acid from the biological sample is at least 70%.
72. The method of any one of items 61-71, wherein the target nucleic acid is amplified with a lower Ct value as compared to otherwise similar samples in water and/or TE buffer.
73. The method of any one of items 59-72, wherein the treatment composition and the crude biological sample are mixed at a ratio of about 0.5:1 to about 4:1, or at a ratio of about 1:1 to about 2:1, with component amounts of the composition being scaled accordingly for other mixture ratios.
74. The method of any one of items 59-73, wherein the biological sample makes up about 10% to 60% of the volume of the mixture of step (a).
75. The method of any one of items 59-74, further comprising incubating the mixture of step (a) at about room temperature for a time interval prior to step (b).
76. The method of item 75, wherein the time interval is up to about 96 hours, and wherein the mixture remains stable through the duration of the time interval.
77. The method of any one of items 59-76, wherein step (b) comprises temperature treating the mixture of (a).
78. The method of item 77, wherein step (b) comprises temperature treating the mixture at a first temperature and then a second temperature.
79. The method of item 78, wherein the first temperature and the second temperature differ by at least 15° C.
80. The method of item 78 or item 79, wherein the first temperature comprises a temperature between 20° to 70° C. and the second temperature comprises a temperature between 85 to 100° C.
81. The method of item 80, wherein the first temperature is room temperature, or about 25° C.
82. The method of any one of items 78-81, wherein (b) comprises heating the mixture at the first temperature for a first time interval and the second temperature for a second time interval.
83. The method of item 82, wherein the first time interval has a duration that depends on the first temperature, the duration being longer for relatively lower temperatures and shorter for relatively higher temperatures.
84. The method of item 82 or item 83, wherein the first and second time intervals are each at least two minutes.
85. The method of any one of items 78-84, wherein the second time interval is no longer than 15 minutes.
86. The method of any one of items 78-85, wherein (b) further comprises incubating the mixture at a third temperature for a third time interval of time prior to performing step (c).
87. The method of item 86, wherein the third temperature is a temperature between 2° to 8° C.
88. The method of item 87, wherein the third interval of time is for at least 1 minute.
89. The method of item 88, wherein the mixture is stored at the third temperature for up to 24 hours prior to performing step (c).
90. The method of any one of items 86-89, further comprising incubating the mixture at a fourth temperature prior to step (c).
91. The method of item 90, wherein the fourth temperature is about 25° C. or room temperature.
92. The method of item 90 or item 91, wherein the first temperature and the fourth temperature are substantially the same.
93. The method of any one of items 90-92, wherein the mixture is stable at the fourth temperature for at least 4 hours.
94. The method of item 93, wherein the mixture is stable at the fourth temperature for at least 96 hours.
95. The method of any one of items 59-94, wherein steps (a) through (c) are performed in a single reaction vessel or tube.
96. The method of any one of items 59-94, wherein steps (a) and (b) are performed in a first reaction vessel or tube and step (c) is performed in a second reaction vessel or tube.
97. The method of item 96, wherein an aliquot of the mixture from (b) is transferred to the second reaction vessel and further diluted prior to performing step (c).
98. The method of item 97, wherein the aliquot of the mixture from (b) is mixed with one or more PCR reagents in the second reaction vessel.
99. The method of any one of items 59-98, wherein (b) comprises adding a protease inhibitor mixture having a plurality of protease inhibitors to mixture (a).
100. The method of any one of items 59-99, wherein the target nucleic acid is a viral target nucleic acid.
101. The method of any one of items 59-100, wherein the target nucleic acid is a SARS-CoV-2 target nucleic acid.
102. The method of any one of items 59-101, wherein the biological sample comprises one or more of a saliva sample, buccal sample, nasal sample, nasal pharyngeal sample, blood sample, urine sample, or semen sample.
103. The method of any one of items 59-102, wherein the biological sample is a resuspension of a dry swab.
104. The method of item 103, wherein the resuspension has a small resuspension volume of about 0.1 ml to about 1 ml.
105. The method of any one of items 59-104, further comprising diagnosing an infection in an organism from which the biological sample was obtained.
106. The method of item 105, wherein the organism is a mammal.
107. The method of item 106, wherein the mammal is a human.
108. The method of any one of items 105-107, wherein the infection is associated with a virus.
109. The method of item 108, wherein the virus is SARS-CoV-2.
110. The method of any one of items 59-109, wherein a plurality of biological samples from different individuals are pooled together to form a multi-individual biological sample, and wherein the multi-individual biological sample is utilized in steps (a) through (c).
111. The method of item 110, wherein, upon detection of the target nucleic acid within the multi-individual biological sample, one or more of the plurality of biological samples that made up the multi-individual biological sample are utilized in steps (a) through (c).
112. The method of any one of items 59-111, wherein the entire method is performed at a point of service.
113. The method of item 112, wherein the biological sample is collected at the point of service.
114. The method of item 112 or item 113, wherein the point of service is a place of public gathering.
115. The method of any one of items 112-114, wherein the point of service is a testing site, medical facility, school, transportation center such as an airport, stadium, arena, or event center.
116. A kit for detecting a target nucleic acid in a biological sample, the kit comprising: the composition of any one of items 1-49; and a PCR reagent mixture.
117. The kit of item 116, wherein the PCR reagent mixture comprises at least one primer and/or at least one probe.
118. The kit of item 117, wherein the PCR reagent mixture includes primers corresponding to a plurality of target nucleic acids.
119. The kit of any one of items 116-118, wherein the PCR reagent mixture includes a TaqMan Fast Virus 1-Step Master Mix or a TaqPath 1-Step RT-qPCR Master Mix.
120. The kit of any one of items 116-119, wherein at least one of the components of the kit is dried or freeze dried.
121. The kit of any one of items 116-120, wherein the kit further comprises a sample collection device.
122. The kit of item 121, wherein the sample collection device is a dry swab collection device.
123. The kit of item 122, wherein the dry swab collection device is a spun polyester swab collection device, a flocked swab non-breakable collection device, or a flocked swab, breakable collection device.
124. The kit of item 121, wherein the sample collection device is a saliva collection container.
125. The kit of any one of items 116-124, further including a PCR array with multiple loci.
126. The kit of item 125, wherein the different loci comprise a well, channel, groove, cavity, site or feature on the surface of the array.
127. A kit for detecting a target nucleic acid in a biological sample, the kit comprising: the composition of any one of items 1-49; and a sample collection device.
128. The kit of item 127, wherein the sample collection device is a dry swab collection device.
129. The kit of item 128, wherein the dry swab collection device is a spun polyester swab collection device, a flocked swab non-breakable collection device, or a flocked swab, breakable collection device.
130. The kit of item 127, wherein the sample collection device is a saliva collection container.
131. The kit of any one of items 127-130, wherein the kit enables a user to self-collect the biological sample.

1. A composition formulated for mixing with a crude biological sample to enable subsequent analysis of a target nucleic acid, the composition comprising:
a surfactant;
a protease component;
a chelating agent; and
a buffering salt.

2. The composition of claim 1, further comprising a saccharide.

3. The composition of claim 2, wherein the saccharide is a disaccharide.

4. The composition of claim 3, wherein the disaccharide comprises sucrose, trehalose, or both.

5. The composition of claim 2, wherein the saccharide is included at a concentration of about 200 mM to about 600 mM.

6. The composition of claim 1, wherein the surfactant comprises a nonionic detergent.

7. The composition of claim 6, wherein the nonionic detergent comprises one or more of nonyl phenoxypolyethoxylethanol (NP-40), TERGITOL 15-S-9, TRITON X-100, or TWEEN 20.

8. The composition of claim 1, wherein the surfactant comprises a cationic detergent.

9. The composition of claim 8, wherein the cationic detergent comprises one or more of benzalkonium chloride (BZK) or didodecyldimethylammonium bromide (DDAB).

10. The composition of claim 1, wherein the surfactant comprises a zwitterionic detergent.

11. The composition of claim 10, wherein the zwitterionic detergent comprises one or more of lauryldimethylamine oxide (LDAO), EMPIGEN BB, or ZWITTERGENT 3-14.

12. The composition of claim 1, wherein the protease component comprises proteinase K.

13. The composition of claim 1, wherein the protease component comprises a mixture of proteases.

14. The composition of claim 13, wherein the mixture of proteases comprises a mixture of proteases isolated from a Streptomyces culture.

15. The composition of claim 14, wherein the protease component comprises pronase.

16. The composition of claim 1, wherein the buffering salt comprises a sodium salt, such as sodium citrate.

17. The composition of claim 16, wherein the sodium salt comprises sodium citrate.

18. The composition of claim 1, wherein the buffering salt comprises a chloride salt.

19. The composition of claim 18, wherein the chloride salt comprises Tris-HCl.

20. The composition of claim 1, wherein the chelating agent comprises ethylenediaminetetraacetic acid (EDTA) or a conjugate base or salt thereof.

21-131. (canceled)


SEQUENCE LISTING (1319 sequences)

SEQ ID NO: 1 - DNA, 2001 bp, Severe acute respiratory syndrome coronavirus 2
tgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctgg

SEQ ID NO: 2 - DNA, 2001 bp, Severe acute respiratory syndrome coronavirus 2
tgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtg

SEQ ID NO: 3 - DNA, 1284 bp, Severe acute respiratory syndrome coronavirus 2
tgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcaga

SEQ ID NO: 4 - DNA, 25 bp, Artificial Sequence
  Synthetic oligonucleotide
tccagcatgtcacaattcagaagta

SEQ ID NO: 5 - DNA, 25 bp, Artificial Sequence
  Synthetic oligonucleotide
gttggatggaaagtgagttcagagt

SEQ ID NO: 6 - DNA, 25 bp, Artificial Sequence
  Synthetic oligonucleotide
cgtagtcgcaacagttcaagaaatt

SEQ ID NO: 7 - DNA, 25 bp, Artificial Sequence
  Synthetic oligonucleotide
tgctgcagcttattatgtgggttat

SEQ ID NO: 8 - DNA, 24 bp, Artificial Sequence
  Synthetic oligonucleotide
tgtctctgggaccaatggtactaa

SEQ ID NO: 9 - DNA, 21 bp, Artificial Sequence
  Synthetic oligonucleotide
gggaacgtggttgacctacac

SEQ ID NO: 10 - DNA, 20 bp, Artificial Sequence
  Synthetic oligonucleotide
ctgtgcacttgaccctctct

SEQ ID NO: 11 - DNA, 24 bp, Artificial Sequence
  Synthetic oligonucleotide
gcagagacagaagaaacagcaaac

SEQ ID NO: 12 - DNA, 24 bp, Artificial Sequence
  Synthetic oligonucleotide
tccaatgttacttggttccatgct

SEQ ID NO: 13 - DNA, 21 bp, Artificial Sequence
  Synthetic oligonucleotide
gccaacaacaacaaggccaaa

SEQ ID NO: 14 - DNA, 23 bp, Artificial Sequence
  Synthetic oligonucleotide
gtgctagcgctaacataggttgt

SEQ ID NO: 15 - DNA, 23 bp, Artificial Sequence
  Synthetic oligonucleotide
caagcctcttctcgttcctcatc

SEQ ID NO: 16 - DNA, 23 bp, Artificial Sequence
  Synthetic oligonucleotide
aaattcaactccaggcagcagta

SEQ ID NO: 17 - DNA, 23 bp, Artificial Sequence
  Synthetic oligonucleotide
ccctcagattcaactggcagtaa

SEQ ID NO: 18 - DNA, 20 bp, Artificial Sequence
  Synthetic oligonucleotide
cttggttcaccgctctcact

SEQ ID NO: 19 - DNA, 21 bp, Artificial Sequence
  Synthetic oligonucleotide
cgcaatcctgctaacaatgct

SEQ ID NO: 20 - DNA, 22 bp, Artificial Sequence
  Synthetic oligonucleotide
cttcagcgttcttcggaatgtc

SEQ ID NO: 21 - DNA, 22 bp, Artificial Sequence
  Synthetic oligonucleotide
gttgcgtcaccaaatgaatgca

SEQ ID NO: 22 - DNA, 23 bp, Artificial Sequence
  Synthetic oligonucleotide
gagcatagtcttgccgaatacca

SEQ ID NO: 23 - DNA, 25 bp, Artificial Sequence
  Synthetic oligonucleotide
gctgcagcttattatgtgggttatc

SEQ ID NO: 24 - DNA, 25 bp, Artificial Sequence
  Synthetic oligonucleotide
cgaagacccagtccctacttattgt

SEQ ID NO: 25 - DNA, 21 bp, Artificial Sequence
  Synthetic oligonucleotide
gcagtcaagcctcttctcgtt

SEQ ID NO: 26 - DNA, 22 bp, Artificial Sequence
  Synthetic oligonucleotide
caagtgtgcctattgggttcca

SEQ ID NO: 27 - DNA, 25 bp, Artificial Sequence
  Synthetic oligonucleotide
cgtactgccactaaagcatacaatg

SEQ ID NO: 28 - DNA, 26 bp, Artificial Sequence
  Synthetic oligonucleotide
acgcctattaatttagtgcgtgatct

SEQ ID NO: 29 - DNA, 24 bp, Artificial Sequence
  Synthetic oligonucleotide
tgaaggaaaacagggtaatttcaa

SEQ ID NO: 30 - DNA, 25 bp, Artificial Sequence
  Synthetic oligonucleotide
cgtcaccaaatgaatgcaaccaaat

SEQ ID NO: 31 - DNA, 20 bp, Artificial Sequence
  Synthetic oligonucleotide
ggttcaccgctctcactcaa

SEQ ID NO: 32 - DNA, 25 bp, Artificial Sequence
  Synthetic oligonucleotide
tgttaacaactaaacgaacaatgtt

SEQ ID NO: 33 - DNA, 21 bp, Artificial Sequence
  Synthetic oligonucleotide
ttcgtggtggtgacggtaaaa

SEQ ID NO: 34 - DNA, 20 bp, Artificial Sequence
  Synthetic oligonucleotide
agaggctgctcgtgttgtac

SEQ ID NO: 35 - DNA, 24 bp, Artificial Sequence
  Synthetic oligonucleotide
ccctcagattcaactggcagtaac

SEQ ID NO: 36 - DNA, 17 bp, Artificial Sequence
  Synthetic oligonucleotide
gcagaggcggcagtcaa

SEQ ID NO: 37 - DNA, 24 bp, Artificial Sequence
  Synthetic oligonucleotide
gtccagaacaaacccaaggaaatt

SEQ ID NO: 38 - DNA, 22 bp, Artificial Sequence
  Synthetic oligonucleotide
caagcctcttctcgttcctcat

SEQ ID NO: 39 - DNA, 24 bp, Artificial Sequence
  Synthetic oligonucleotide
ttgacagattgaaccagcttgaga

SEQ ID NO: 40 - DNA, 20 bp, Artificial Sequence
  Synthetic oligonucleotide
ggttcaccgctctcactcaa

SEQ ID NO: 41 - DNA, 23 bp, Artificial Sequence
  Synthetic oligonucleotide
gggcgattttgttaaagccactt

SEQ ID NO: 42 - DNA, 22 bp, Artificial Sequence
  Synthetic oligonucleotide
ctgctgctgaggcttctaagaa

SEQ ID NO: 43 - DNA, 25 bp, Artificial Sequence
  Synthetic oligonucleotide
gccccaaggtttacccaataatact

SEQ ID NO: 44 - DNA, 22 bp, Artificial Sequence
  Synthetic oligonucleotide
gtctatccagttgcgtcaccaa

SEQ ID NO: 45 - DNA, 22 bp, Artificial Sequence
  Synthetic oligonucleotide
gcagacgggcgattttgttaaa

SEQ ID NO: 46 - DNA, 23 bp, Artificial Sequence
  Synthetic oligonucleotide
aactgagggagccttgaatacac

SEQ ID NO: 47 - DNA, 22 bp, Artificial Sequence
  Synthetic oligonucleotide
ttcgaagacccagtccctactt

SEQ ID NO: 48 - DNA, 22 bp, Artificial Sequence
  Synthetic oligonucleotide
tgctgtagactgtgcacttgac

SEQ ID NO: 49 - DNA, 22 bp, Artificial Sequence
  Synthetic oligonucleotide
tgcaatcgtgctacaacttcct

SEQ ID NO: 50 - DNA, 25 bp, Artificial Sequence
  Synthetic oligonucleotide
gttgccataacaagtgtgcctattg

SEQ ID NO: 51 - DNA, 20 bp, Artificial Sequence
  Synthetic oligonucleotide
gcagacgggcgattttgtta

SEQ ID NO: 52 - DNA, 21 bp, Artificial Sequence
  Synthetic oligonucleotide
tgctgcaatcgtgctacaact

SEQ ID NO: 53 - DNA, 22 bp, Artificial Sequence
  Synthetic oligonucleotide
ggctactaccgaagagctacca

SEQ ID NO: 54 - DNA, 23 bp, Artificial Sequence
  Synthetic oligonucleotide
ggacaaggcgttccaattaacac

SEQ ID NO: 55 - DNA, 29 bp, Artificial Sequence
  Synthetic oligonucleotide
gatccatttttgggtgtttattaccacaa

SEQ ID NO: 56 - DNA, 19 bp, Artificial Sequence
  Synthetic oligonucleotide
cgtcggccccaaggtttac

SEQ ID NO: 57 - DNA, 24 bp, Artificial Sequence
  Synthetic oligonucleotide
cttctcgttcctcatcacgtagtc

SEQ ID NO: 58 - DNA, 22 bp, Artificial Sequence
  Synthetic oligonucleotide
caacaacaaggccaaactgtca

SEQ ID NO: 59 - DNA, 25 bp, Artificial Sequence
  Synthetic oligonucleotide
agcttgagagcaaaatgtctggtaa

SEQ ID NO: 60 - DNA, 23 bp, Artificial Sequence
  Synthetic oligonucleotide
gcagagacagaagaaacagcaaa

SEQ ID NO: 61 - DNA, 25 bp, Artificial Sequence
  Synthetic oligonucleotide
gtccagatgaccaaattggctacta

SEQ ID NO: 62 - DNA, 22 bp, Artificial Sequence
  Synthetic oligonucleotide
tcacgtagtcgcaacagttcaa

SEQ ID NO: 63 - DNA, 22 bp, Artificial Sequence
  Synthetic oligonucleotide
gagggagccttgaatacaccaa

SEQ ID NO: 64 - DNA, 24 bp, Artificial Sequence
  Synthetic oligonucleotide
cagcaaactgtgactcttcttcct

SEQ ID NO: 65 - DNA, 25 bp, Artificial Sequence
  Synthetic oligonucleotide
acaaggccaaactgtcactaagaaa

SEQ ID NO: 66 - DNA, 25 bp, Artificial Sequence
  Synthetic oligonucleotide
ctcaattaccccctgcatacactaa

SEQ ID NO: 67 - DNA, 20 bp, Artificial Sequence
  Synthetic oligonucleotide
ccacgtgctagcgctaacat

SEQ ID NO: 68 - DNA, 25 bp, Artificial Sequence
  Synthetic oligonucleotide
ggagccttgaatacaccaaaagatc

SEQ ID NO: 69 - DNA, 19 bp, Artificial Sequence
  Synthetic oligonucleotide
gcagacgtggtccagaaca

SEQ ID NO: 70 - DNA, 25 bp, Artificial Sequence
  Synthetic oligonucleotide
aaaatgtctggtaaaggccaacaac

SEQ ID NO: 71 - DNA, 19 bp, Artificial Sequence
  Synthetic oligonucleotide
ggcgcgatcaaaacaacgt

SEQ ID NO: 72 - DNA, 25 bp, Artificial Sequence
  Synthetic oligonucleotide
ggaccaatggtactaagaggtttga

SEQ ID NO: 73 - DNA, 21 bp, Artificial Sequence
  Synthetic oligonucleotide
ggtcgcactattgcctttgga

SEQ ID NO: 74 - DNA, 24 bp, Artificial Sequence
  Synthetic oligonucleotide
ctggtgattcttcttcaggttgga

SEQ ID NO: 75 - DNA, 22 bp, Artificial Sequence
  Synthetic oligonucleotide
atcacgtagtcgcaacagttca

SEQ ID NO: 76 - DNA, 24 bp, Artificial Sequence
  Synthetic oligonucleotide
caacaaggccaaactgtcactaag

SEQ ID NO: 77 - DNA, 25 bp, Artificial Sequence
  Synthetic oligonucleotide
cctctctcagaaacaaagtgtacgt

SEQ ID NO: 78 - DNA, 19 bp, Artificial Sequence
  Synthetic oligonucleotide
cagcgcttcagcgttcttc

SEQ ID NO: 79 - DNA, 23 bp, Artificial Sequence
  Synthetic oligonucleotide
gtttggtggaccctcagattcaa

SEQ ID NO: 80 - DNA, 24 bp, Artificial Sequence
  Synthetic oligonucleotide
ttggaggctgtgtgttctcttatg

SEQ ID NO: 81 - DNA, 21 bp, Artificial Sequence
  Synthetic oligonucleotide
ccccaaaatcagcgaaatgca

SEQ ID NO: 82 - DNA, 21 bp, Artificial Sequence
  Synthetic oligonucleotide
cgtgatctccctcagggtttt

SEQ ID NO: 83 - DNA, 25 bp, Artificial Sequence
  Synthetic oligonucleotide
gactaaagaaggtgccactacttgt

SEQ ID NO: 84 - DNA, 23 bp, Artificial Sequence
  Synthetic oligonucleotide
ctacacaggtgccatcaaattgg

SEQ ID NO: 85 - DNA, 23 bp, Artificial Sequence
  Synthetic oligonucleotide
ggcatcatatgggttgcaactga

SEQ ID NO: 86 - DNA, 21 bp, Artificial Sequence
  Synthetic oligonucleotide
agacgtggtccagaacaaacc

SEQ ID NO: 87 - DNA, 19 bp, Artificial Sequence
  Synthetic oligonucleotide
tggccgcaaattgcacaat

SEQ ID NO: 88 - DNA, 21 bp, Artificial Sequence
  Synthetic oligonucleotide
aaatctgctgctgaggcttct

SEQ ID NO: 89 - DNA, 26 bp, Artificial Sequence
  Synthetic oligonucleotide
ggctttagaaccattggtagatttgc

SEQ ID NO: 90 - DNA, 24 bp, Artificial Sequence
  Synthetic oligonucleotide
caaaatgtctggtaaaggccaaca

SEQ ID NO: 91 - DNA, 25 bp, Artificial Sequence
  Synthetic oligonucleotide
caaaatgtctggtaaaggccaacaa

SEQ ID NO: 92 - DNA, 20 bp, Artificial Sequence
  Synthetic oligonucleotide
ctcttgctttgctgctgctt

SEQ ID NO: 93 - DNA, 19 bp, Artificial Sequence
  Synthetic oligonucleotide
cgggaacgtggttgaccta

SEQ ID NO: 94 - DNA, 25 bp, Artificial Sequence
  Synthetic oligonucleotide
gttacttaccccaaaatgctgttgt

SEQ ID NO: 95 - DNA, 24 bp, Artificial Sequence
  Synthetic oligonucleotide
aacaccaatagcagtccagatgac

SEQ ID NO: 96 - DNA, 25 bp, Artificial Sequence
  Synthetic oligonucleotide
ggctgtgtgttctcttatgttggtt

SEQ ID NO: 97 - DNA, 23 bp, Artificial Sequence
  Synthetic oligonucleotide
agattgaaccagcttgagagcaa

SEQ ID NO: 98 - DNA, 26 bp, Artificial Sequence
  Synthetic oligonucleotide
caacaaaagttggatggaaagtgagt

SEQ ID NO: 99 - DNA, 22 bp, Artificial Sequence
  Synthetic oligonucleotide
tcatcacgtagtcgcaacagtt

SEQ ID NO: 100 - DNA, 27 bp, Artificial Sequence
  Synthetic oligonucleotide
aggacttgttcttacctttcttttcca

SEQ ID NO: 101 - DNA, 25 bp, Artificial Sequence
  Synthetic oligonucleotide
agagctatgaattgcagacaccttt

SEQ ID NO: 102 - DNA, 21 bp, Artificial Sequence
  Synthetic oligonucleotide
ggtgatgctgctcttgctttg

SEQ ID NO: 103 - DNA, 22 bp, Artificial Sequence
  Synthetic oligonucleotide
ggtggaccctcagattcaactg

SEQ ID NO: 104 - DNA, 18 bp, Artificial Sequence
  Synthetic oligonucleotide
gcggcagtcaagcctctt

SEQ ID NO: 105 - DNA, 24 bp, Artificial Sequence
  Synthetic oligonucleotide
aacaaggccaaactgtcactaaga

SEQ ID NO: 106 - DNA, 30 bp, Artificial Sequence
  Synthetic oligonucleotide
tgagaagtctaacataataagaggctggat

SEQ ID NO: 107 - DNA, 22 bp, Artificial Sequence
  Synthetic oligonucleotide
cgtcaccaaatgaatgcaacca

SEQ ID NO: 108 - DNA, 25 bp, Artificial Sequence
  Synthetic oligonucleotide
gaagtaggacctgagcatagtcttg

SEQ ID NO: 109 - DNA, 19 bp, Artificial Sequence
  Synthetic oligonucleotide
accccgcattacgtttggt

SEQ ID NO: 110 - DNA, 25 bp, Artificial Sequence
  Synthetic oligonucleotide
ggtggtgacggtaaaatgaaagatc

SEQ ID NO: 111 - DNA, 26 bp, Artificial Sequence
  Synthetic oligonucleotide
gcaaagaaatttgacaccttcaatgg

SEQ ID NO: 112 - DNA, 23 bp, Artificial Sequence
  Synthetic oligonucleotide
gcgaattttgtggcactgagaat

SEQ ID NO: 113 - DNA, 25 bp, Artificial Sequence
  Synthetic oligonucleotide
ggcttgaaaaccattcttcgtaagg

SEQ ID NO: 114 - DNA, 23 bp, Artificial Sequence
  Synthetic oligonucleotide
aatctgctgctgaggcttctaag

SEQ ID NO: 115 - DNA, 24 bp, Artificial Sequence
  Synthetic oligonucleotide
gggagccttgaatacaccaaaaga

SEQ ID NO: 116 - DNA, 24 bp, Artificial Sequence
  Synthetic oligonucleotide
caatcgtgctacaacttcctcaag

SEQ ID NO: 117 - DNA, 25 bp, Artificial Sequence
  Synthetic oligonucleotide
accaaatgtgcctttcaactctcat

SEQ ID NO: 118 - DNA, 18 bp, Artificial Sequence
  Synthetic oligonucleotide
tggcagacgggcgatttt

SEQ ID NO: 119 - DNA, 21 bp, Artificial Sequence
  Synthetic oligonucleotide
ggttcaccgctctcactcaac

SEQ ID NO: 120 - DNA, 22 bp, Artificial Sequence
  Synthetic oligonucleotide
ttcgtggtggtgacggtaaaat

SEQ ID NO: 121 - DNA, 26 bp, Artificial Sequence
  Synthetic oligonucleotide
aacagagttgttatttctagtgatgt

SEQ ID NO: 122 - DNA, 23 bp, Artificial Sequence
  Synthetic oligonucleotide
cattcccaccaacagagcctaaa

SEQ ID NO: 123 - DNA, 23 bp, Artificial Sequence
  Synthetic oligonucleotide
caatcctgctaacaatgctgcaa

SEQ ID NO: 124 - DNA, 22 bp, Artificial Sequence
  Synthetic oligonucleotide
agacgggcgattttgttaaagc

SEQ ID NO: 125 - DNA, 22 bp, Artificial Sequence
  Synthetic oligonucleotide
gcacttgaccctctctcagaaa

SEQ ID NO: 126 - DNA, 25 bp, Artificial Sequence
  Synthetic oligonucleotide
caaggcgttccaattaacaccaata

SEQ ID NO: 127 - DNA, 19 bp, Artificial Sequence
  Synthetic oligonucleotide
ggcagacgtggtccagaac

SEQ ID NO: 128 - DNA, 25 bp, Artificial Sequence
  Synthetic oligonucleotide
ggaccttgaaggaaaacagggtaat

SEQ ID NO: 129 - DNA, 19 bp, Artificial Sequence
  Synthetic oligonucleotide
agggtggtcgcactattgc

SEQ ID NO: 130 - DNA, 25 bp, Artificial Sequence
  Synthetic oligonucleotide
ctgctgcagatttggatgatttctc

SEQ ID NO: 131 - DNA, 27 bp, Artificial Sequence
  Synthetic oligonucleotide
acaaaagttggatggaaagtgagttca

SEQ ID NO: 132 - DNA, 23 bp, Artificial Sequence
  Synthetic oligonucleotide
acatgtctctgggaccaatggta

SEQ ID NO: 133 - DNA, 25 bp, Artificial Sequence
  Synthetic oligonucleotide
ctcagccttttcttatggaccttga

SEQ ID NO: 134 - DNA, 18 bp, Artificial Sequence
  Synthetic oligonucleotide
ccagcgcttcagcgttct

SEQ ID NO: 135 - DNA, 24 bp, Artificial Sequence
  Synthetic oligonucleotide
tcaaggaacaacattgccaaaagg

SEQ ID NO: 136 - DNA, 25 bp, Artificial Sequence
  Synthetic oligonucleotide
ccaaatgtgcctttcaactctcatg

SEQ ID NO: 137 - DNA, 23 bp, Artificial Sequence
  Synthetic oligonucleotide
gcagagacagaagaaacagcaaa

SEQ ID NO: 138 - DNA, 24 bp, Artificial Sequence
  Synthetic oligonucleotide
agcttgagagcaaaatgtctggta

SEQ ID NO: 139 - DNA, 22 bp, Artificial Sequence
  Synthetic oligonucleotide
ccgaagagctaccagacgaatt

SEQ ID NO: 140 - DNA, 24 bp, Artificial Sequence
  Synthetic oligonucleotide
gttgccataacaagtgtgcctatt

SEQ ID NO: 141 - DNA, 19 bp, Artificial Sequence
  Synthetic oligonucleotide
ggttcaccgctctcactca

SEQ ID NO: 142 - DNA, 20 bp, Artificial Sequence
  Synthetic oligonucleotide
gatgctgctcttgctttgct

SEQ ID NO: 143 - DNA, 21 bp, Artificial Sequence
  Synthetic oligonucleotide
ggtggtcgcactattgccttt

SEQ ID NO: 144 - DNA, 22 bp, Artificial Sequence
  Synthetic oligonucleotide
tgctgcttgacagattgaacca

SEQ ID NO: 145 - DNA, 24 bp, Artificial Sequence
  Synthetic oligonucleotide
gcgttccaattaacaccaatagca

SEQ ID NO: 146 - DNA, 20 bp, Artificial Sequence
  Synthetic oligonucleotide
gggaacgtggttgacctaca

SEQ ID NO: 147 - DNA, 22 bp, Artificial Sequence
  Synthetic oligonucleotide
cttgcgaattttgtggcactga

SEQ ID NO: 148 - DNA, 23 bp, Artificial Sequence
  Synthetic oligonucleotide
tggcagtaaccagaatggagaac

SEQ ID NO: 149 - DNA, 23 bp, Artificial Sequence
  Synthetic oligonucleotide
tcactcaacatggcaaggaagac

SEQ ID NO: 150 - DNA, 24 bp, Artificial Sequence
  Synthetic oligonucleotide
ggccccaaggtttacccaataata

SEQ ID NO: 151 - DNA, 24 bp, Artificial Sequence
  Synthetic oligonucleotide
cacgtggtgtttattaccctgaca

SEQ ID NO: 152 - DNA, 26 bp, Artificial Sequence
  Synthetic oligonucleotide
ggctttatgggtagaattcgatctgt

SEQ ID NO: 153 - DNA, 21 bp, Artificial Sequence
  Synthetic oligonucleotide
cctattgggttccacgtgcta

SEQ ID NO: 154 - DNA, 25 bp, Artificial Sequence
  Synthetic oligonucleotide
catacaaaacattcccaccaacaga

SEQ ID NO: 155 - DNA, 17 bp, Artificial Sequence
  Synthetic oligonucleotide
tgcaccccgcattacgt

SEQ ID NO: 156 - DNA, 23 bp, Artificial Sequence
  Synthetic oligonucleotide
gcaaccaaatgtgcctttcaact

SEQ ID NO: 157 - DNA, 18 bp, Artificial Sequence
  Synthetic oligonucleotide
ggaactgggccagaagct

SEQ ID NO: 158 - DNA, 21 bp, Artificial Sequence
  Synthetic oligonucleotide
ggtgatgctgctcttgctttg

SEQ ID NO: 159 - DNA, 19 bp, Artificial Sequence
  Synthetic oligonucleotide
ttggccgcaaattgcacaa

SEQ ID NO: 160 - DNA, 25 bp, Artificial Sequence
  Synthetic oligonucleotide
tgcctggaatattggtgaacagaaa

SEQ ID NO: 161 - DNA, 24 bp, Artificial Sequence
  Synthetic oligonucleotide
aactcaattaccccctgcatacac

SEQ ID NO: 162 - DNA, 21 bp, Artificial Sequence
  Synthetic oligonucleotide
gctgcaatcgtgctacaactt

SEQ ID NO: 163 - DNA, 25 bp, Artificial Sequence
  Synthetic oligonucleotide
caaaatgtctggtaaaggccaacaa

SEQ ID NO: 164 - DNA, 24 bp, Artificial Sequence
  Synthetic oligonucleotide
ccaatagcagtccagatgaccaaa

SEQ ID NO: 165 - DNA, 26 bp, Artificial Sequence
  Synthetic oligonucleotide
cccctgcatacactaattctttcaca

SEQ ID NO: 166 - DNA, 19 bp, Artificial Sequence
  Synthetic oligonucleotide
ccttcgggaacgtggttga

SEQ ID NO: 167 - DNA, 22 bp, Artificial Sequence
  Synthetic oligonucleotide
attcaactccaggcagcagtag

SEQ ID NO: 168 - DNA, 18 bp, Artificial Sequence
  Synthetic oligonucleotide
gggcgcgatcaaaacaac

SEQ ID NO: 169 - DNA, 19 bp, Artificial Sequence
  Synthetic oligonucleotide
cgcattggcatggaagtca

SEQ ID NO: 170 - DNA, 22 bp, Artificial Sequence
  Synthetic oligonucleotide
cattcccaccaacagagcctaa

SEQ ID NO: 171 - DNA, 23 bp, Artificial Sequence
  Synthetic oligonucleotide
gaaatctgctgctgaggcttcta

SEQ ID NO: 172 - DNA, 21 bp, Artificial Sequence
  Synthetic oligonucleotide
ggtccagaacaaacccaagga

SEQ ID NO: 173 - DNA, 21 bp, Artificial Sequence
  Synthetic oligonucleotide
ttggcatggaagtcacacctt

SEQ ID NO: 174 - DNA, 23 bp, Artificial Sequence
  Synthetic oligonucleotide
cgtcaccaaatgaatgcaaccaa

SEQ ID NO: 175 - DNA, 23 bp, Artificial Sequence
  Synthetic oligonucleotide
ctcaattaccccctgcatacact

SEQ ID NO: 176 - DNA, 21 bp, Artificial Sequence
  Synthetic oligonucleotide
cgtggtggtgacggtaaaatg

SEQ ID NO: 177 - DNA, 22 bp, Artificial Sequence
  Synthetic oligonucleotide
ctggacttccctatggtgctaa

SEQ ID NO: 178 - DNA, 22 bp, Artificial Sequence
  Synthetic oligonucleotide
accgcagagacagaagaaacag

SEQ ID NO: 179 - DNA, 23 bp, Artificial Sequence
  Synthetic oligonucleotide
gcacttgaccctctctcagaaac

SEQ ID NO: 180 - DNA, 23 bp, Artificial Sequence
  Synthetic oligonucleotide
cacttgaccctctctcagaaaca

SEQ ID NO: 181 - DNA, 21 bp, Artificial Sequence
  Synthetic oligonucleotide
cttcagcgttcttcggaatgt

SEQ ID NO: 182 - DNA, 17 bp, Artificial Sequence
  Synthetic oligonucleotide
tggcagacgggcgattt

SEQ ID NO: 183 - DNA, 24 bp, Artificial Sequence
  Synthetic oligonucleotide
tgattacaaacattggccgcaaat

SEQ ID NO: 184 - DNA, 24 bp, Artificial Sequence
  Synthetic oligonucleotide
tctggcttgaaaaccattcttcgt

SEQ ID NO: 185 - DNA, 25 bp, Artificial Sequence
  Synthetic oligonucleotide
ccccctgcatacactaattctttca

SEQ ID NO: 186 - DNA, 26 bp, Artificial Sequence
  Synthetic oligonucleotide
cggctttagaaccattggtagatttg

SEQ ID NO: 187 - DNA, 20 bp, Artificial Sequence
  Synthetic oligonucleotide
ctcaagccttaccgcagaga

SEQ ID NO: 188 - DNA, 22 bp, Artificial Sequence
  Synthetic oligonucleotide
gcagtccagatgaccaaattgg

SEQ ID NO: 189 - DNA, 29 bp, Artificial Sequence
  Synthetic oligonucleotide
gaagttatttgactcctggtgattcttct

SEQ ID NO: 190 - DNA, 23 bp, Artificial Sequence
  Synthetic oligonucleotide
tgattacaaacattggccgcaaa

SEQ ID NO: 191 - DNA, 25 bp, Artificial Sequence
  Synthetic oligonucleotide
gaccttaaattccctcgaggacaag

SEQ ID NO: 192 - DNA, 22 bp, Artificial Sequence
  Synthetic oligonucleotide
tggtggaccctcagattcaact

SEQ ID NO: 193 - DNA, 25 bp, Artificial Sequence
  Synthetic oligonucleotide
gggagccttgaatacaccaaaagat

SEQ ID NO: 194 - DNA, 18 bp, Artificial Sequence
  Synthetic oligonucleotide
gcacccgcaatcctgcta

SEQ ID NO: 195 - DNA, 21 bp, Artificial Sequence
  Synthetic oligonucleotide
aaatctgctgctgaggcttct

SEQ ID NO: 196 - DNA, 25 bp, Artificial Sequence
  Synthetic oligonucleotide
ccaggaactaatcagacaaggaact

SEQ ID NO: 197 - DNA, 17 bp, Artificial Sequence
  Synthetic oligonucleotide
ttggcacccgcaatcct

SEQ ID NO: 198 - DNA, 24 bp, Artificial Sequence
  Synthetic oligonucleotide
ggtccagaacaaacccaaggaaat

SEQ ID NO: 199 - DNA, 21 bp, Artificial Sequence
  Synthetic oligonucleotide
gtcaagcctcttctcgttcct

SEQ ID NO: 200 - DNA, 20 bp, Artificial Sequence
  Synthetic oligonucleotide
gcagacgtggtccagaacaa

SEQ ID NO: 201 - DNA, 24 bp, Artificial Sequence
  Synthetic oligonucleotide
tgggaccaatggtactaagaggtt

SEQ ID NO: 202 - DNA, 25 bp, Artificial Sequence
  Synthetic oligonucleotide
aacaacattgccaaaaggcttctac

SEQ ID NO: 203 - DNA, 21 bp, Artificial Sequence
  Synthetic oligonucleotide
ccgaagagctaccagacgaat

SEQ ID NO: 204 - DNA, 23 bp, Artificial Sequence
  Synthetic oligonucleotide
cgtcaccaaatgaatgcaaccaa

SEQ ID NO: 205 - DNA, 23 bp, Artificial Sequence
  Synthetic oligonucleotide
ctggtgctgcagcttattatgtg

SEQ ID NO: 206 - DNA, 20 bp, Artificial Sequence
  Synthetic oligonucleotide
cttgctttgctgctgcttga

SEQ ID NO: 207 - DNA, 23 bp, Artificial Sequence
  Synthetic oligonucleotide
acacaggtgccatcaaattggat

SEQ ID NO: 208 - DNA, 21 bp, Artificial Sequence
  Synthetic oligonucleotide
catgtctctgggaccaatggt

SEQ ID NO: 209 - DNA, 22 bp, Artificial Sequence
  Synthetic oligonucleotide
ccctcagattcaactggcagta

SEQ ID NO: 210 - DNA, 23 bp, Artificial Sequence
  Synthetic oligonucleotide
gaaccagcttgagagcaaaatgt

SEQ ID NO: 211 - DNA, 17 bp, Artificial Sequence
  Synthetic oligonucleotide
gcaatggcggtgatgct

SEQ ID NO: 212 - DNA, 23 bp, Artificial Sequence
  Synthetic oligonucleotide
ttcttcctgctgcagatttggat

SEQ ID NO: 213 - DNA, 25 bp, Artificial Sequence
  Synthetic oligonucleotide
gaacaacattgccaaaaggcttcta

SEQ ID NO: 214 - DNA, 27 bp, Artificial Sequence
  Synthetic oligonucleotide
caactcaggacttgttcttacctttct

SEQ ID NO: 215 - DNA, 24 bp, Artificial Sequence
  Synthetic oligonucleotide
gggaatgtccaaattttgtatttc

SEQ ID NO: 216 - DNA, 22 bp, Artificial Sequence
  Synthetic oligonucleotide
gctgctgcttgacagattgaac

SEQ ID NO: 217 - DNA, 25 bp, Artificial Sequence
  Synthetic oligonucleotide
ttgaccctctctcagaaacaaagtg

SEQ ID NO: 218 - DNA, 23 bp, Artificial Sequence
  Synthetic oligonucleotide
gctgatgaaactcaagccttacc

SEQ ID NO: 219 - DNA, 25 bp, Artificial Sequence
  Synthetic oligonucleotide
ctctcagccttttcttatggacctt

SEQ ID NO: 220 - DNA, 28 bp, Artificial Sequence
  Synthetic oligonucleotide
gaggctggatttttggtactactttaga

SEQ ID NO: 221 - DNA, 23 bp, Artificial Sequence
  Synthetic oligonucleotide
agatgctgtagactgtgcacttg

SEQ ID NO: 222 - DNA, 19 bp, Artificial Sequence
  Synthetic oligonucleotide
gctttgctgctgcttgaca

SEQ ID NO: 223 - DNA, 28 bp, Artificial Sequence
  Synthetic oligonucleotide
cacttttgaatatgtctctcagcctttt

SEQ ID NO: 224 - DNA, 23 bp, Artificial Sequence
  Synthetic oligonucleotide
tgcaaccaaatgtgcctttcaac

SEQ ID NO: 225 - DNA, 20 bp, Artificial Sequence
  Synthetic oligonucleotide
gctagaatggctggcaatgg

SEQ ID NO: 226 - DNA, 24 bp, Artificial Sequence
  Synthetic oligonucleotide
acgtggtgtttattaccctgacaa

SEQ ID NO: 227 - DNA, 23 bp, Artificial Sequence
  Synthetic oligonucleotide
attcccaccaacagagcctaaaa

SEQ ID NO: 228 - DNA, 26 bp, Artificial Sequence
  Synthetic oligonucleotide
cttgatggctttatgggtagaattcg

SEQ ID NO: 229 - DNA, 25 bp, Artificial Sequence
  Synthetic oligonucleotide
cgtggtgtttattaccctgacaaag

SEQ ID NO: 230 - DNA, 24 bp, Artificial Sequence
  Synthetic oligonucleotide
ctgctgcagatttggatgatttct

SEQ ID NO: 231 - DNA, 21 bp, Artificial Sequence
  Synthetic oligonucleotide
cccgcaatcctgctaacaatg

SEQ ID NO: 232 - DNA, 24 bp, Artificial Sequence
  Synthetic oligonucleotide
gctaacaaagacggcatcatatgg

SEQ ID NO: 233 - DNA, 20 bp, Artificial Sequence
  Synthetic oligonucleotide
acggcatcatatgggttgca

SEQ ID NO: 234 - DNA, 21 bp, Artificial Sequence
  Synthetic oligonucleotide
tcaccgctctcactcaacatg

SEQ ID NO: 235 - DNA, 23 bp, Artificial Sequence
  Synthetic oligonucleotide
actcttcttcctgctgcagattt

SEQ ID NO: 236 - DNA, 23 bp, Artificial Sequence
  Synthetic oligonucleotide
ctggtaaaggccaacaacaacaa

SEQ ID NO: 237 - DNA, 25 bp, Artificial Sequence
  Synthetic oligonucleotide
cagagacagaagaaacagcaaactg

SEQ ID NO: 238 - DNA, 19 bp, Artificial Sequence
  Synthetic oligonucleotide
ttccacgtgctagcgctaa

SEQ ID NO: 239 - DNA, 18 bp, Artificial Sequence
  Synthetic oligonucleotide
ggcagacgtggtccagaa

SEQ ID NO: 240 - DNA, 19 bp, Artificial Sequence
  Synthetic oligonucleotide
gcacccgcaatcctgctaa

SEQ ID NO: 241 - DNA, 25 bp, Artificial Sequence
  Synthetic oligonucleotide
acttccctatggtgctaacaaagac

SEQ ID NO: 242 - DNA, 23 bp, Artificial Sequence
  Synthetic oligonucleotide
ccttaccgcagagacagaagaaa

SEQ ID NO: 243 - DNA, 25 bp, Artificial Sequence
  Synthetic oligonucleotide
gttaaagccacttgcgaattttgtg

SEQ ID NO: 244 - DNA, 22 bp, Artificial Sequence
  Synthetic oligonucleotide
acaacaaggccaaactgtcact

SEQ ID NO: 245 - DNA, 25 bp, Artificial Sequence
  Synthetic oligonucleotide
tgaaaagagctatgaattgcagaca

SEQ ID NO: 246 - DNA, 21 bp, Artificial Sequence
  Synthetic oligonucleotide
ccgcaatcctgctaacaatgc

SEQ ID NO: 247 - DNA, 23 bp, Artificial Sequence
  Synthetic oligonucleotide
aatgcaaccaaatgtgcctttca

SEQ ID NO: 248 - DNA, 24 bp, Artificial Sequence
  Synthetic oligonucleotide
ctcagattcaactggcagtaacca

SEQ ID NO: 249 - DNA, 24 bp, Artificial Sequence
  Synthetic oligonucleotide
tgctgcagcttattatgtgggtta

SEQ ID NO: 250 - DNA, 24 bp, Artificial Sequence
  Synthetic oligonucleotide
accaaatgtgcctttcaactctca

SEQ ID NO: 251 - DNA, 20 bp, Artificial Sequence
  Synthetic oligonucleotide
cgtaagggtggtcgcactat

SEQ ID NO: 252 - DNA, 26 bp, Artificial Sequence
  Synthetic oligonucleotide
aaaatgtgtgagctttcatgaagcat

SEQ ID NO: 253 - DNA, 24 bp, Artificial Sequence
  Synthetic oligonucleotide
ggaatggctaaagacaagaccaat

SEQ ID NO: 254 - DNA, 30 bp, Artificial Sequence
  Synthetic oligonucleotide
caagcttaacaactgaaattcaaatcaaca

SEQ ID NO: 255 - DNA, 24 bp, Artificial Sequence
  Synthetic oligonucleotide
gggcaaatatggaaacatacgtga

SEQ ID NO: 256 - DNA, 18 bp, Artificial Sequence
  Synthetic oligonucleotide
tgtcgccccaagcaactt

SEQ ID NO: 257 - DNA, 27 bp, Artificial Sequence
  Synthetic oligonucleotide
aaaatgtgtgagctttcatgaagcatt

SEQ ID NO: 258-266 - skipped (9 entries)

SEQ ID NO: 267 - DNA, 19 bp, Artificial Sequence
  Synthetic oligonucleotide
gcgaccacccttacgaaga

SEQ ID NO: 268 - DNA, 23 bp, Artificial Sequence
  Synthetic oligonucleotide
ccaatttgatggcacctgtgtag

SEQ ID NO: 269 - DNA, 30 bp, Artificial Sequence
  Synthetic oligonucleotide
aataaccatcaatattcttaaacacaaatt

SEQ ID NO: 270 - DNA, 26 bp, Artificial Sequence
  Synthetic oligonucleotide
aattctacccataaagccatcaagct

SEQ ID NO: 271 - DNA, 22 bp, Artificial Sequence
  Synthetic oligonucleotide
cgtctggtagctcttcggtagt

SEQ ID NO: 272 - DNA, 20 bp, Artificial Sequence
  Synthetic oligonucleotide
aagcagcagcaaagcaagag

SEQ ID NO: 273 - DNA, 25 bp, Artificial Sequence
  Synthetic oligonucleotide
attgtatgctttagtggcagtacgt

SEQ ID NO: 274 - DNA, 17 bp, Artificial Sequence
  Synthetic oligonucleotide
gggccgacgttgttttg

SEQ ID NO: 275 - DNA, 27 bp, Artificial Sequence
  Synthetic oligonucleotide
taaagttagaagtttgatagattcctt

SEQ ID NO: 276 - DNA, 25 bp, Artificial Sequence
  Synthetic oligonucleotide
gcttgagtttcatcagccttcttct

SEQ ID NO: 277 - DNA, 24 bp, Artificial Sequence
  Synthetic oligonucleotide
acccaataggcacacttgttatgg

SEQ ID NO: 278 - DNA, 19 bp, Artificial Sequence
  Synthetic oligonucleotide
acgtctgccgaaagcttgt

SEQ ID NO: 279 - DNA, 20 bp, Artificial Sequence
  Synthetic oligonucleotide
ggctctgttggtgggaatgt

SEQ ID NO: 280 - DNA, 21 bp, Artificial Sequence
  Synthetic oligonucleotide
agggactgggtcttcgaatct

SEQ ID NO: 281 - DNA, 22 bp, Artificial Sequence
  Synthetic oligonucleotide
gcacacttgttatggcaaccaa

SEQ ID NO: 282 - DNA, 25 bp, Artificial Sequence
  Synthetic oligonucleotide
caagtagtggcaccttctttagtca

SEQ ID NO: 283 - DNA, 26 bp, Artificial Sequence
  Synthetic oligonucleotide
ggtaataaacaccacgtgtgaaagaa

SEQ ID NO: 284 - DNA, 23 bp, Artificial Sequence
  Synthetic oligonucleotide
tgccgtctttgttagcaccatag

SEQ ID NO: 285 - DNA, 26 bp, Artificial Sequence
  Synthetic oligonucleotide
taaagttagaagtttgatagattcct

SEQ ID NO: 286 - DNA, 21 bp, Artificial Sequence
  Synthetic oligonucleotide
ggctctgttggtgggaatgtt

SEQ ID NO: 287 - DNA, 19 bp, Artificial Sequence
  Synthetic oligonucleotide
ttagcgctagcacgtggaa

SEQ ID NO: 288 - DNA, 24 bp, Artificial Sequence
  Synthetic oligonucleotide
atttggacattccccattgaaggt

SEQ ID NO: 289 - DNA, 24 bp, Artificial Sequence
  Synthetic oligonucleotide
cgtctggtagctcttcggtagtag

SEQ ID NO: 290 - DNA, 25 bp, Artificial Sequence
  Synthetic oligonucleotide
catgagagttgaaaggcacatttgg

SEQ ID NO: 291 - DNA, 22 bp, Artificial Sequence
  Synthetic oligonucleotide
ggtcatctggactgctattggt

SEQ ID NO: 292 - DNA, 22 bp, Artificial Sequence
  Synthetic oligonucleotide
acccacataataagctgcagca

SEQ ID NO: 293 - DNA, 28 bp, Artificial Sequence
  Synthetic oligonucleotide
gatttcaacgtacactttgtttctgaga

SEQ ID NO: 294 - DNA, 18 bp, Artificial Sequence
  Synthetic oligonucleotide
catgccaatgcgcgacat

SEQ ID NO: 295 - DNA, 25 bp, Artificial Sequence
  Synthetic oligonucleotide
ttgttggcctttaccagacattttg

SEQ ID NO: 296 - DNA, 22 bp, Artificial Sequence
  Synthetic oligonucleotide
gccttttggcaatgttgttcct

SEQ ID NO: 297 - DNA, 23 bp, Artificial Sequence
  Synthetic oligonucleotide
tgcgttctccattctggttactg

SEQ ID NO: 298 - DNA, 22 bp, Artificial Sequence
  Synthetic oligonucleotide
cgggtgccaatgtgatcttttg

SEQ ID NO: 299 - DNA, 24 bp, Artificial Sequence
  Synthetic oligonucleotide
agctgtccaacctgaagaagaatc

SEQ ID NO: 300 - DNA, 24 bp, Artificial Sequence
  Synthetic oligonucleotide
ctgagggagatcacgcactaaatt

SEQ ID NO: 301 - DNA, 25 bp, Artificial Sequence
  Synthetic oligonucleotide
tctcaagctggttcaatctgtcaag

SEQ ID NO: 302 - DNA, 22 bp, Artificial Sequence
  Synthetic oligonucleotide
tctgccgaaagcttgtgttaca

SEQ ID NO: 303 - DNA, 24 bp, Artificial Sequence
  Synthetic oligonucleotide
agcaggaagaagagtcacagtttg

SEQ ID NO: 304 - DNA, 21 bp, Artificial Sequence
  Synthetic oligonucleotide
tctgcggtaaggcttgagttt

SEQ ID NO: 305 - DNA, 23 bp, Artificial Sequence
  Synthetic oligonucleotide
gccttttggcaatgttgttcctt

SEQ ID NO: 306 - DNA, 21 bp, Artificial Sequence
  Synthetic oligonucleotide
cccgtctgccatgaagtttca

SEQ ID NO: 307 - DNA, 27 bp, Artificial Sequence
  Synthetic oligonucleotide
cgcactagaataaactctgaactcact

SEQ ID NO: 308 - DNA, 21 bp, Artificial Sequence
  Synthetic oligonucleotide
gccttgtcctcgagggaattt

SEQ ID NO: 309 - DNA, 22 bp, Artificial Sequence
  Synthetic oligonucleotide
tcatccaaatctgcagcaggaa

SEQ ID NO: 310 - DNA, 20 bp, Artificial Sequence
  Synthetic oligonucleotide
gggccgacgttgttttgatc

SEQ ID NO: 311 - DNA, 24 bp, Artificial Sequence
  Synthetic oligonucleotide
gattgcagcattgttagcaggatt

SEQ ID NO: 312 - DNA, 24 bp, Artificial Sequence
  Synthetic oligonucleotide
gccgaaagcttgtgttacattgta

SEQ ID NO: 313 - DNA, 23 bp, Artificial Sequence
  Synthetic oligonucleotide
aacccatatgatgccgtctttgt

SEQ ID NO: 314 - DNA, 25 bp, Artificial Sequence
  Synthetic oligonucleotide
ctgagggagatcacgcactaaatta

SEQ ID NO: 315 - DNA, 22 bp, Artificial Sequence
  Synthetic oligonucleotide
catgttgagtgagagcggtgaa

SEQ ID NO: 316 - DNA, 22 bp, Artificial Sequence
  Synthetic oligonucleotide
agctggttcaatctgtcaagca

SEQ ID NO: 317 - DNA, 19 bp, Artificial Sequence
  Synthetic oligonucleotide
gtgcgaccacccttacgaa

SEQ ID NO: 318 - DNA, 25 bp, Artificial Sequence
  Synthetic oligonucleotide
gcactaaattaataggcgtgtgctt

SEQ ID NO: 319 - DNA, 24 bp, Artificial Sequence
  Synthetic oligonucleotide
gcaaccaacataagagaacacaca

SEQ ID NO: 320 - DNA, 25 bp, Artificial Sequence
  Synthetic oligonucleotide
acgaagaatggttttcaagccagat

SEQ ID NO: 321 - DNA, 24 bp, Artificial Sequence
  Synthetic oligonucleotide
cgcaactggatagacagatcgaat

SEQ ID NO: 322 - DNA, 24 bp, Artificial Sequence
  Synthetic oligonucleotide
gcaagactatgctcaggtcctact

SEQ ID NO: 323 - DNA, 25 bp, Artificial Sequence
  Synthetic oligonucleotide
acaagtagtggcaccttctttagtc

SEQ ID NO: 324 - DNA, 22 bp, Artificial Sequence
  Synthetic oligonucleotide
aggtcttccttgccatgttgag

SEQ ID NO: 325 - DNA, 22 bp, Artificial Sequence
  Synthetic oligonucleotide
agagggtcaagtgcacagtcta

SEQ ID NO: 326 - DNA, 19 bp, Artificial Sequence
  Synthetic oligonucleotide
tgcgcgacattccgaagaa

SEQ ID NO: 327 - DNA, 24 bp, Artificial Sequence
  Synthetic oligonucleotide
tcgcaagtggctttaacaaaatcg

SEQ ID NO: 328 - DNA, 22 bp, Artificial Sequence
  Synthetic oligonucleotide
tcagcactgctcatggattgtt

SEQ ID NO: 329 - DNA, 23 bp, Artificial Sequence
  Synthetic oligonucleotide
tgctgcctggagttgaatttctt

SEQ ID NO: 330 - DNA, 21 bp, Artificial Sequence
  Synthetic oligonucleotide
ctctgcggtaaggcttgagtt

SEQ ID NO: 331 - DNA, 22 bp, Artificial Sequence
  Synthetic oligonucleotide
gggtaagtaaccacaagtagtg

SEQ ID NO: 332 - DNA, 22 bp, Artificial Sequence
  Synthetic oligonucleotide
gcgaccacccttacgaagaatg

SEQ ID NO: 333 - DNA, 25 bp, Artificial Sequence
  Synthetic oligonucleotide
tctttgttagcaccatagggaagtc

SEQ ID NO: 334 - DNA, 25 bp, Artificial Sequence
  Synthetic oligonucleotide
ccaatttggtcatctggactgctat

SEQ ID NO: 335 - DNA, 21 bp, Artificial Sequence
  Synthetic oligonucleotide
tgttgagtgagagcggtgaac

SEQ ID NO: 336 - DNA, 25 bp, Artificial Sequence
  Synthetic oligonucleotide
gtcatctggactgctattggtgtta

SEQ ID NO: 337 - DNA, 25 bp, Artificial Sequence
  Synthetic oligonucleotide
gtaggacagggttatcaaacctctt

SEQ ID NO: 338 - DNA, 20 bp, Artificial Sequence
  Synthetic oligonucleotide
aagcagcagcaaagcaagag

SEQ ID NO: 339 - DNA, 22 bp, Artificial Sequence
  Synthetic oligonucleotide
tttaccgtcaccaccacgaatt

SEQ ID NO: 340 - DNA, 23 bp, Artificial Sequence
  Synthetic oligonucleotide
ttcttgaactgttgcgactacgt

SEQ ID NO: 341 - DNA, 25 bp, Artificial Sequence
  Synthetic oligonucleotide
cctttaccagacattttgctctcaa

SEQ ID NO: 342 - DNA, 25 bp, Artificial Sequence
  Synthetic oligonucleotide
tgtatgctttagtggcagtacgttt

SEQ ID NO: 343 - DNA, 23 bp, Artificial Sequence
  Synthetic oligonucleotide
ttcctggtccccaaaatttcctt

SEQ ID NO: 344 - DNA, 21 bp, Artificial Sequence
  Synthetic oligonucleotide
cctgtgtaggtcaaccacgtt

SEQ ID NO: 345 - DNA, 19 bp, Artificial Sequence
  Synthetic oligonucleotide
cacgttcccgaaggtgtga

SEQ ID NO: 346 - DNA, 24 bp, Artificial Sequence
  Synthetic oligonucleotide
ctacgtgatgaggaacgagaagag

SEQ ID NO: 347 - DNA, 24 bp, Artificial Sequence
  Synthetic oligonucleotide
caggaagaagagtcacagtttgct

SEQ ID NO: 348 - DNA, 26 bp, Artificial Sequence
  Synthetic oligonucleotide
tctaccaatggttctaaagccgaaaa

SEQ ID NO: 349 - DNA, 23 bp, Artificial Sequence
  Synthetic oligonucleotide
ggaacccaataggcacacttgtt

SEQ ID NO: 350 - DNA, 23 bp, Artificial Sequence
  Synthetic oligonucleotide
ccacataataagctgcagcacca

SEQ ID NO: 351 - DNA, 22 bp, Artificial Sequence
  Synthetic oligonucleotide
gccttgtcctcgagggaattta

SEQ ID NO: 352 - DNA, 20 bp, Artificial Sequence
  Synthetic oligonucleotide
tggcccagttcctaggtagt

SEQ ID NO: 353 - DNA, 22 bp, Artificial Sequence
  Synthetic oligonucleotide
gagggtcaagtgcacagtctac

SEQ ID NO: 354 - DNA, 21 bp, Artificial Sequence
  Synthetic oligonucleotide
caccatagggaagtccagctt

SEQ ID NO: 355 - DNA, 25 bp, Artificial Sequence
  Synthetic oligonucleotide
tttgtcagggtaataaacaccacgt

SEQ ID NO: 356 - DNA, 24 bp, Artificial Sequence
  Synthetic oligonucleotide
caagactatgctcaggtcctactt

SEQ ID NO: 357 - DNA, 22 bp, Artificial Sequence
  Synthetic oligonucleotide
ttttaccgtcaccaccacgaat

SEQ ID NO: 358 - DNA, 21 bp, Artificial Sequence
  Synthetic oligonucleotide
gcaaagcaagagcagcatcac

SEQ ID NO: 359 - DNA, 24 bp, Artificial Sequence
  Synthetic oligonucleotide
ccctgttttccttcaaggtccata

SEQ ID NO: 360 - DNA, 25 bp, Artificial Sequence
  Synthetic oligonucleotide
caagactatgctcaggtcctacttc

SEQ ID NO: 361 - DNA, 25 bp, Artificial Sequence
  Synthetic oligonucleotide
caaggtccataagaaaaggctgaga

SEQ ID NO: 362 - DNA, 25 bp, Artificial Sequence
  Synthetic oligonucleotide
ctgattagttcctggtccccaaaat

SEQ ID NO: 363 - DNA, 25 bp, Artificial Sequence
  Synthetic oligonucleotide
ttgtatgctttagtggcagtacgtt

SEQ ID NO: 364 - DNA, 25 bp, Artificial Sequence
  Synthetic oligonucleotide
gtgggaatgttttgtatgcgtcaat

SEQ ID NO: 365 - DNA, 20 bp, Artificial Sequence
  Synthetic oligonucleotide
cgttcccgaaggtgtgactt

SEQ ID NO: 366 - DNA, 22 bp, Artificial Sequence
  Synthetic oligonucleotide
gtctgccgaaagcttgtgttac

SEQ ID NO: 367 - DNA, 16 bp, Artificial Sequence
  Synthetic oligonucleotide
cgccccactgcgttct

SEQ ID NO: 368 - DNA, 24 bp, Artificial Sequence
  Synthetic oligonucleotide
agctcttcggtagtagccaatttg

SEQ ID NO: 369 - DNA, 25 bp, Artificial Sequence
  Synthetic oligonucleotide
gtcctcgagggaatttaaggtcttc

SEQ ID NO: 370 - DNA, 24 bp, Artificial Sequence
  Synthetic oligonucleotide
ccttgaggaagttgtagcacgatt

SEQ ID NO: 371 - DNA, 22 bp, Artificial Sequence
  Synthetic oligonucleotide
gttgcgactacgtgatgaggaa

SEQ ID NO: 372 - DNA, 24 bp, Artificial Sequence
  Synthetic oligonucleotide
gcggccaatgtttgtaatcagttc

SEQ ID NO: 373 - DNA, 21 bp, Artificial Sequence
  Synthetic oligonucleotide
ccgtctgccatgaagtttcac

SEQ ID NO: 374 - DNA, 22 bp, Artificial Sequence
  Synthetic oligonucleotide
aatctgtcaagcagcagcaaag

SEQ ID NO: 375 - DNA, 25 bp, Artificial Sequence
  Synthetic oligonucleotide
gcacatttggttgcattcatttggt

SEQ ID NO: 376 - DNA, 25 bp, Artificial Sequence
  Synthetic oligonucleotide
ccatatgatgccgtctttgttagca

SEQ ID NO: 377 - DNA, 23 bp, Artificial Sequence
  Synthetic oligonucleotide
tggcctttaccagacattttgct

SEQ ID NO: 378 - DNA, 25 bp, Artificial Sequence
  Synthetic oligonucleotide
gccgaaagcttgtgttacattgtat

SEQ ID NO: 379 - DNA, 21 bp, Artificial Sequence
  Synthetic oligonucleotide
ttttaccgtcaccaccacgaa

SEQ ID NO: 380 - DNA, 26 bp, Artificial Sequence
  Synthetic oligonucleotide
ggcaaatctaccaatggttctaaagc

SEQ ID NO: 381 - DNA, 25 bp, Artificial Sequence
  Synthetic oligonucleotide
ctttgtttctgagagagggtcaagt

SEQ ID NO: 382 - DNA, 23 bp, Artificial Sequence
  Synthetic oligonucleotide
gccttgtcctcgagggaatttaa

SEQ ID NO: 383 - DNA, 24 bp, Artificial Sequence
  Synthetic oligonucleotide
tgaaattaccctgttttccttcaa

SEQ ID NO: 384 - DNA, 25 bp, Artificial Sequence
  Synthetic oligonucleotide
tttgtttctgagagagggtcaagtg

SEQ ID NO: 385 - DNA, 23 bp, Artificial Sequence
  Synthetic oligonucleotide
cgtagaagccttttggcaatgtt

SEQ ID NO: 386 - DNA, 21 bp, Artificial Sequence
  Synthetic oligonucleotide
actgcgttctccattctggtt

SEQ ID NO: 387 - DNA, 28 bp, Artificial Sequence
  Synthetic oligonucleotide
gtggtaataaacacccaaaaatggatca

SEQ ID NO: 388 - DNA, 21 bp, Artificial Sequence
  Synthetic oligonucleotide
ttctggcccagttcctaggta

SEQ ID NO: 389 - DNA, 19 bp, Artificial Sequence
  Synthetic oligonucleotide
caaccacgttcccgaaggt

SEQ ID NO: 390 - DNA, 22 bp, Artificial Sequence
  Synthetic oligonucleotide
cacgattgcagcattgttagca

SEQ ID NO: 391 - DNA, 25 bp, Artificial Sequence
  Synthetic oligonucleotide
agcagcagatttcttagtgacagtt

SEQ ID NO: 392 - DNA, 19 bp, Artificial Sequence
  Synthetic oligonucleotide
caaggctccctcagttgca

SEQ ID NO: 393 - DNA, 19 bp, Artificial Sequence
  Synthetic oligonucleotide
ttaggcctgagttgagtca

SEQ ID NO: 394 - DNA, 25 bp, Artificial Sequence
  Synthetic oligonucleotide
cggtagtagccaatttggtcatctg

SEQ ID NO: 395 - DNA, 23 bp, Artificial Sequence
  Synthetic oligonucleotide
agcctcagcagcagatttcttag

SEQ ID NO: 396 - DNA, 25 bp, Artificial Sequence
  Synthetic oligonucleotide
cgaagaatggttttcaagccagatt

SEQ ID NO: 397 - DNA, 21 bp, Artificial Sequence
  Synthetic oligonucleotide
ctgagggagatcacgcactaa

SEQ ID NO: 398 - DNA, 20 bp, Artificial Sequence
  Synthetic oligonucleotide
aggctctgttggtgggaatg

SEQ ID NO: 399 - DNA, 21 bp, Artificial Sequence
  Synthetic oligonucleotide
ggtgacgcaactggatagaca

SEQ ID NO: 400 - DNA, 26 bp, Artificial Sequence
  Synthetic oligonucleotide
agtcctaggttgaagataacccacat

SEQ ID NO: 401 - DNA, 23 bp, Artificial Sequence
  Synthetic oligonucleotide
cttccttgccatgttgagtgaga

SEQ ID NO: 402 - DNA, 22 bp, Artificial Sequence
  Synthetic oligonucleotide
tgaactgttgcgactacgtgat

SEQ ID NO: 403 - DNA, 28 bp, Artificial Sequence
  Synthetic oligonucleotide
cccttggttgaatagtcttgattatgga

SEQ ID NO: 404 - DNA, 23 bp, Artificial Sequence
  Synthetic oligonucleotide
ctctgttggtgggaatgttttgt

SEQ ID NO: 405 - DNA, 24 bp, Artificial Sequence
  Synthetic oligonucleotide
caatttgcggccaatgtttgtaat

SEQ ID NO: 406 - DNA, 25 bp, Artificial Sequence
  Synthetic oligonucleotide
gggtaataaacaccacgtgtgaaag

SEQ ID NO: 407 - DNA, 23 bp, Artificial Sequence
  Synthetic oligonucleotide
ccttgaggaagttgtagcacgat

SEQ ID NO: 408 - DNA, 24 bp, Artificial Sequence
  Synthetic oligonucleotide
tggtcccagagacatgtatagcat

SEQ ID NO: 409 - DNA, 25 bp, Artificial Sequence
  Synthetic oligonucleotide
agactatgctcaggtcctacttctg

SEQ ID NO: 410 - DNA, 25 bp, Artificial Sequence
  Synthetic oligonucleotide
aagactatgctcaggtcctacttct

SEQ ID NO: 411 - DNA, 25 bp, Artificial Sequence
  Synthetic oligonucleotide
ctgaagaagaatcaccaggagtcaa

SEQ ID NO: 412 - DNA, 25 bp, Artificial Sequence
  Synthetic oligonucleotide
ccttttggcaatgttgttccttgag

SEQ ID NO: 413 - DNA, 20 bp, Artificial Sequence
  Synthetic oligonucleotide
gcgggtgccaatgtgatctt

SEQ ID NO: 414 - DNA, 22 bp, Artificial Sequence
  Synthetic oligonucleotide
ttcggaaccttctccaacaaca

SEQ ID NO: 415 - DNA, 23 bp, Artificial Sequence
  Synthetic oligonucleotide
acagtttggccttgttgttgttg

SEQ ID NO: 416 - DNA, 22 bp, Artificial Sequence
  Synthetic oligonucleotide
agtgccacaaaattcgcaagtg

SEQ ID NO: 417 - DNA, 26 bp, Artificial Sequence
  Synthetic oligonucleotide
cagtggaagcaaaataaacaccatca

SEQ ID NO: 418 - DNA, 23 bp, Artificial Sequence
  Synthetic oligonucleotide
cactgcgttctccattctggtta

SEQ ID NO: 419 - DNA, 25 bp, Artificial Sequence
  Synthetic oligonucleotide
ccctgttttccttcaaggtccataa

SEQ ID NO: 420 - DNA, 23 bp, Artificial Sequence
  Synthetic oligonucleotide
gctctcaagctggttcaatctgt

SEQ ID NO: 421 - DNA, 24 bp, Artificial Sequence
  Synthetic oligonucleotide
aaaggcacatttggttgcattcat

SEQ ID NO: 422 - DNA, 21 bp, Artificial Sequence
  Synthetic oligonucleotide
cccactgcgttctccattctg

SEQ ID NO: 423 - DNA, 23 bp, Artificial Sequence
  Synthetic oligonucleotide
acgcacagaattttgagcagttt

SEQ ID NO: 424 - DNA, 21 bp, Artificial Sequence
  Synthetic oligonucleotide
agaacacacagcctccaaagg

SEQ ID NO: 425 - DNA, 25 bp, Artificial Sequence
  Synthetic oligonucleotide
aatttgcggccaatgtttgtaatca

SEQ ID NO: 426 - DNA, 23 bp, Artificial Sequence
  Synthetic oligonucleotide
tttcttctgtctctgcggtaagg

SEQ ID NO: 427 - DNA, 23 bp, Artificial Sequence
  Synthetic oligonucleotide
cctcagcagcagatttcttagtg

SEQ ID NO: 428 - DNA, 24 bp, Artificial Sequence
  Synthetic oligonucleotide
gctgtccaacctgaagaagaatca

SEQ ID NO: 429 - DNA, 23 bp, Artificial Sequence
  Synthetic oligonucleotide
cggtaaggcttgagtttcatcag

SEQ ID NO: 430 - DNA, 20 bp, Artificial Sequence
  Synthetic oligonucleotide
gcgaccacccttacgaagaa

SEQ ID NO: 431 - DNA, 22 bp, Artificial Sequence
  Synthetic oligonucleotide
ctggcccagttcctaggtagta

SEQ ID NO: 432 - DNA, 25 bp, Artificial Sequence
  Synthetic oligonucleotide
ggcccagttcctaggtagtagaaat

SEQ ID NO: 433 - DNA, 21 bp, Artificial Sequence
  Synthetic oligonucleotide
cactgctcatggattgttgca

SEQ ID NO: 434 - DNA, 20 bp, Artificial Sequence
  Synthetic oligonucleotide
cccgtctgccatgaagtttc

SEQ ID NO: 435 - DNA, 24 bp, Artificial Sequence
  Synthetic oligonucleotide
tttggtcatctggactgctattgg

SEQ ID NO: 436 - DNA, 21 bp, Artificial Sequence
  Synthetic oligonucleotide
tgcggtaaggcttgagtttca

SEQ ID NO: 437 - DNA, 24 bp, Artificial Sequence
  Synthetic oligonucleotide
gtagaagccttttggcaatgttgt

SEQ ID NO: 438 - DNA, 24 bp, Artificial Sequence
  Synthetic oligonucleotide
ggactgggtcttcgaatctaaagt

SEQ ID NO: 439 - DNA, 24 bp, Artificial Sequence
  Synthetic oligonucleotide
gtagggactgggtcttcgaatcta

SEQ ID NO: 440 - DNA, 25 bp, Artificial Sequence
  Synthetic oligonucleotide
ggtgaaccaagacgcagtattattg

SEQ ID NO: 441 - DNA, 25 bp, Artificial Sequence
  Synthetic oligonucleotide
aggtccataagaaaaggctgagaga

SEQ ID NO: 442 - DNA, 21 bp, Artificial Sequence
  Synthetic oligonucleotide
cgactacgtgatgaggaacga

SEQ ID NO: 443 - DNA, 24 bp, Artificial Sequence
  Synthetic oligonucleotide
agggtaataaacaccacgtgtgaa

SEQ ID NO: 444 - DNA, 26 bp, Artificial Sequence
  Synthetic oligonucleotide
cacgtgtgaaagaattagtgtatgca

SEQ ID NO: 445 - DNA, 25 bp, Artificial Sequence
  Synthetic oligonucleotide
ctgcctggagttgaatttcttgaac

SEQ ID NO: 446 - DNA, 22 bp, Artificial Sequence
  Synthetic oligonucleotide
gtgacgcaactggatagacaga

SEQ ID NO: 447 - DNA, 24 bp, Artificial Sequence
  Synthetic oligonucleotide
ctgcctggagttgaatttcttgaa

SEQ ID NO: 448 - DNA, 23 bp, Artificial Sequence
  Synthetic oligonucleotide
gcctcagcagcagatttcttagt

SEQ ID NO: 449 - DNA, 22 bp, Artificial Sequence
  Synthetic oligonucleotide
tcaagtgcacagtctacagcat

SEQ ID NO: 450 - DNA, 18 bp, Artificial Sequence
  Synthetic oligonucleotide
cgccattgccagccattc

SEQ ID NO: 451 - DNA, 25 bp, Artificial Sequence
  Synthetic oligonucleotide
agggtaataaacaccacgtgtgaaa

SEQ ID NO: 452 - DNA, 22 bp, Artificial Sequence
  Synthetic oligonucleotide
ccttcggaaccttctccaacaa

SEQ ID NO: 453 - DNA, 22 bp, Artificial Sequence
  Synthetic oligonucleotide
tgcggtaaggcttgagtttcat

SEQ ID NO: 454 - DNA, 20 bp, Artificial Sequence
  Synthetic oligonucleotide
accagctgtccaacctgaag

SEQ ID NO: 455 - DNA, 23 bp, Artificial Sequence
  Synthetic oligonucleotide
gccgtctttgttagcaccatagg

SEQ ID NO: 456 - DNA, 24 bp, Artificial Sequence
  Synthetic oligonucleotide
cctcagcagcagatttcttagtga

SEQ ID NO: 457 - DNA, 25 bp, Artificial Sequence
  Synthetic oligonucleotide
gactgggtcttcgaatctaaagtag

SEQ ID NO: 458 - DNA, 22 bp, Artificial Sequence
  Synthetic oligonucleotide
agtgcacagtctacagcatctg

SEQ ID NO: 459 - DNA, 16 bp, Artificial Sequence
  Synthetic oligonucleotide
accttcggaaccttct

SEQ ID NO: 460 - DNA, 22 bp, Artificial Sequence
  Synthetic oligonucleotide
ctccctcagttgcaacccatat

SEQ ID NO: 461 - DNA, 24 bp, Artificial Sequence
  Synthetic oligonucleotide
cttgtcctcgagggaatttaaggt

SEQ ID NO: 462 - DNA, 23 bp, Artificial Sequence
  Synthetic oligonucleotide
tggtgttaattggaacgccttgt

SEQ ID NO: 463 - DNA, 24 bp, Artificial Sequence
  Synthetic oligonucleotide
ccttttggcaatgttgttccttga

SEQ ID NO: 464 - DNA, 20 bp, Artificial Sequence
  Synthetic oligonucleotide
aagcagcagcaaagcaagag

SEQ ID NO: 465 - DNA, 24 bp, Artificial Sequence
  Synthetic oligonucleotide
cttgaggaagttgtagcacgattg

SEQ ID NO: 466 - DNA, 22 bp, Artificial Sequence
  Synthetic oligonucleotide
tcatccaaatctgcagcaggaa

SEQ ID NO: 467 - DNA, 25 bp, Artificial Sequence
  Synthetic oligonucleotide
tctccaacaacacctgtatggttac

SEQ ID NO: 468 - DNA, 23 bp, Artificial Sequence
  Synthetic oligonucleotide
agtgcgggagaaaattgatcgta

SEQ ID NO: 469 - DNA, 23 bp, Artificial Sequence
  Synthetic oligonucleotide
aaaggcacatttggttgcattca

SEQ ID NO: 470 - DNA, 21 bp, Artificial Sequence
  Synthetic oligonucleotide
ggaacgagaagaggcttgact

SEQ ID NO: 471 - DNA, 28 bp, Artificial Sequence
  Synthetic oligonucleotide
gactgggtcttcgaatctaaagtagtac

SEQ ID NO: 472 - DNA, 21 bp, Artificial Sequence
  Synthetic oligonucleotide
ctgcagcaggaagaagagtca

SEQ ID NO: 473 - DNA, 27 bp, Artificial Sequence
  Synthetic oligonucleotide
ttcaacgtacactttgtttctgagaga

SEQ ID NO: 474 - DNA, 21 bp, Artificial Sequence
  Synthetic oligonucleotide
gcgaccacccttacgaagaat

SEQ ID NO: 475 - DNA, 20 bp, Artificial Sequence
  Synthetic oligonucleotide
tctgctcccttctgcgtaga

SEQ ID NO: 476 - DNA, 23 bp, Artificial Sequence
  Synthetic oligonucleotide
gggtaattgagttctggttgtaa

SEQ ID NO: 477 - DNA, 23 bp, Artificial Sequence
  Synthetic oligonucleotide
cttgttgttgttggcctttacca

SEQ ID NO: 478 - DNA, 26 bp, Artificial Sequence
  Synthetic oligonucleotide
acagtgaaggatttcaacgtacactt

SEQ ID NO: 479 - DNA, 25 bp, Artificial Sequence
  Synthetic oligonucleotide
cacaaaattcgcaagtggctttaac

SEQ ID NO: 480 - DNA, 24 bp, Artificial Sequence
  Synthetic oligonucleotide
cagggtaataaacaccacgtgtga

SEQ ID NO: 481 - DNA, 23 bp, Artificial Sequence
  Synthetic oligonucleotide
ccctcagttgcaacccatatgat

SEQ ID NO: 482 - DNA, 23 bp, Artificial Sequence
  Synthetic oligonucleotide
ggtgttaattggaacgccttgtc

SEQ ID NO: 483 - DNA, 25 bp, Artificial Sequence
  Synthetic oligonucleotide
gggtaattgagttctggttgtaaga

SEQ ID NO: 484 - DNA, 22 bp, Artificial Sequence
  Synthetic oligonucleotide
gaactgttgcgactacgtgatg

SEQ ID NO: 485 - DNA, 25 bp, Artificial Sequence
  Synthetic oligonucleotide
acaaaattcgcaagtggctttaaca

SEQ ID NO: 486 - DNA, 23 bp, Artificial Sequence
  Synthetic oligonucleotide
cacataataagctgcagcaccag

SEQ ID NO: 487 - DNA, 18 bp, Artificial Sequence
  Synthetic oligonucleotide
gggccgacgttgttttga

SEQ ID NO: 488 - DNA, 19 bp, Artificial Sequence
  Synthetic oligonucleotide
cctactgctgcctggagtt

SEQ ID NO: 489 - DNA, 19 bp, Artificial Sequence
  Synthetic oligonucleotide
gcgcgacattccgaagaac

SEQ ID NO: 490 - DNA, 18 bp, Artificial Sequence
  Synthetic oligonucleotide
cagcatcaccgccattgc

SEQ ID NO: 491 - DNA, 18 bp, Artificial Sequence
  Synthetic oligonucleotide
cgcctctgctcccttctg

SEQ ID NO: 492 - DNA, 25 bp, Artificial Sequence
  Synthetic oligonucleotide
ctctgaactcactttccatccaact

SEQ ID NO: 493 - DNA, 21 bp, Artificial Sequence
  Synthetic oligonucleotide
gtgtgacttccatgccaatgc

SEQ ID NO: 494 - DNA, 22 bp, Artificial Sequence
  Synthetic oligonucleotide
gtgacagtttggccttgttgtt

SEQ ID NO: 495 - DNA, 22 bp, Artificial Sequence
  Synthetic oligonucleotide
agtgccacaaaattcgcaagtg

SEQ ID NO: 496 - DNA, 21 bp, Artificial Sequence
  Synthetic oligonucleotide
gtctgccgaaagcttgtgtta

SEQ ID NO: 497 - DNA, 20 bp, Artificial Sequence
  Synthetic oligonucleotide
agcacgtggaacccaatagg

SEQ ID NO: 498 - DNA, 22 bp, Artificial Sequence
  Synthetic oligonucleotide
tcggaaccttctccaacaacac

SEQ ID NO: 499 - DNA, 25 bp, Artificial Sequence
  Synthetic oligonucleotide
ggccaatgtttgtaatcagttcctt

SEQ ID NO: 500 - DNA, 25 bp, Artificial Sequence
  Synthetic oligonucleotide
gccatgaagtttcaccacaatgatc

SEQ ID NO: 501 - DNA, 25 bp, Artificial Sequence
  Synthetic oligonucleotide
gcctttaccagacattttgctctca

SEQ ID NO: 502 - DNA, 24 bp, Artificial Sequence
  Synthetic oligonucleotide
tggcaaccaacataagagaacaca

SEQ ID NO: 503 - DNA, 22 bp, Artificial Sequence
  Synthetic oligonucleotide
gcagcattgttagcaggattgc

SEQ ID NO: 504 - DNA, 21 bp, Artificial Sequence
  Synthetic oligonucleotide
ccattgccagccattctagca

SEQ ID NO: 505 - DNA, 25 bp, Artificial Sequence
  Synthetic oligonucleotide
caggattcaggtacatgaccatgag

SEQ ID NO: 506 - DNA, 15 bp, Artificial Sequence
  Synthetic oligonucleotide
acgctgcagtcctcg

SEQ ID NO: 507 - DNA, 24 bp, Artificial Sequence
  Synthetic oligonucleotide
caatcaggagagtcatgcctgtat

SEQ ID NO: 508 - DNA, 27 bp, Artificial Sequence
  Synthetic oligonucleotide
gcgggatcatcatctttttctagaaca

SEQ ID NO: 509 - DNA, 20 bp, Artificial Sequence
  Synthetic oligonucleotide
gcctgtgaacgcgagttaga

SEQ ID NO: 510 - DNA, 26 bp, Artificial Sequence
  Synthetic oligonucleotide
gcctttaccagacattttgctctcaa

SEQ ID NO: 511-519 - skipped (9 entries)

SEQ ID NO: 520 - DNA, 19 bp, Artificial Sequence
  Synthetic oligonucleotide
tagactgtgcacttgaccc

SEQ ID NO: 521 - DNA, 17 bp, Artificial Sequence
  Synthetic oligonucleotide
acgtttggtggaccctc

SEQ ID NO: 522 - DNA, 23 bp, Artificial Sequence
  Synthetic oligonucleotide
atcattacaaaattgaaattcac

SEQ ID NO: 523 - DNA, 18 bp, Artificial Sequence
  Synthetic oligonucleotide
caagacgcagtattattg

SEQ ID NO: 524 - DNA, 17 bp, Artificial Sequence
  Synthetic oligonucleotide
cttctggcccagttcct

SEQ ID NO: 525 - DNA, 16 bp, Artificial Sequence
  Synthetic oligonucleotide
actgccgcctctgctc

SEQ ID NO: 526 - DNA, 21 bp, Artificial Sequence
  Synthetic oligonucleotide
ctggatagacagatcgaattc

SEQ ID NO: 527 - DNA, 22 bp, Artificial Sequence
  Synthetic oligonucleotide
accaggagtcaaataacttcta

SEQ ID NO: 528 - DNA, 19 bp, Artificial Sequence
  Synthetic oligonucleotide
acccttggttgaatagtct

SEQ ID NO: 529 - DNA, 19 bp, Artificial Sequence
  Synthetic oligonucleotide
tcgcactagaataaactct

SEQ ID NO: 530 - DNA, 17 bp, Artificial Sequence
  Synthetic oligonucleotide
tcccaccaacagagcct

SEQ ID NO: 531 - DNA, 19 bp, Artificial Sequence
  Synthetic oligonucleotide
ctgcttgacagattgaacc

SEQ ID NO: 532 - DNA, 22 bp, Artificial Sequence
  Synthetic oligonucleotide
cagccttcttctttttgtcctt

SEQ ID NO: 533 - DNA, 17 bp, Artificial Sequence
  Synthetic oligonucleotide
attgtggtgaaacttca

SEQ ID NO: 534 - DNA, 18 bp, Artificial Sequence
  Synthetic oligonucleotide
ccgtctgccatgaagttt

SEQ ID NO: 535 - DNA, 19 bp, Artificial Sequence
  Synthetic oligonucleotide
cagcatgtcacaattcaga

SEQ ID NO: 536 - DNA, 22 bp, Artificial Sequence
  Synthetic oligonucleotide
ccactgagaagtctaacataat

SEQ ID NO: 537 - DNA, 16 bp, Artificial Sequence
  Synthetic oligonucleotide
actcctggtgattctt

SEQ ID NO: 538 - DNA, 17 bp, Artificial Sequence
  Synthetic oligonucleotide
ccccctgcatacactaa

SEQ ID NO: 539 - DNA, 23 bp, Artificial Sequence
  Synthetic oligonucleotide
atggttacaacctatgttagcgc

SEQ ID NO: 540 - DNA, 17 bp, Artificial Sequence
  Synthetic oligonucleotide
ccagaagctggacttcc

SEQ ID NO: 541 - DNA, 17 bp, Artificial Sequence
  Synthetic oligonucleotide
ctgccagttgaatctga

SEQ ID NO: 542 - DNA, 18 bp, Artificial Sequence
  Synthetic oligonucleotide
atggtgctaacaaagacg

SEQ ID NO: 543 - DNA, 17 bp, Artificial Sequence
  Synthetic oligonucleotide
acatggcaaggaagacc

SEQ ID NO: 544 - DNA, 18 bp, Artificial Sequence
  Synthetic oligonucleotide
taagaggctggatttttg

SEQ ID NO: 545 - DNA, 19 bp, Artificial Sequence
  Synthetic oligonucleotide
tcctcaaggaacaacattg

SEQ ID NO: 546 - DNA, 22 bp, Artificial Sequence
  Synthetic oligonucleotide
ttttgaaattaccctgttttcc

SEQ ID NO: 547 - DNA, 18 bp, Artificial Sequence
  Synthetic oligonucleotide
aaggctccctcagttgca

SEQ ID NO: 548 - DNA, 18 bp, Artificial Sequence
  Synthetic oligonucleotide
ttcctggtccccaaaatt

SEQ ID NO: 549 - DNA, 21 bp, Artificial Sequence
  Synthetic oligonucleotide
cttcaaggtccataagaaaag

SEQ ID NO: 550 - DNA, 17 bp, Artificial Sequence
  Synthetic oligonucleotide
tcctggtccccaaaatt

SEQ ID NO: 551 - DNA, 19 bp, Artificial Sequence
  Synthetic oligonucleotide
tctcagtggaagcaaaata

SEQ ID NO: 552 - DNA, 18 bp, Artificial Sequence
  Synthetic oligonucleotide
caacagttcaagaaattc

SEQ ID NO: 553 - DNA, 19 bp, Artificial Sequence
  Synthetic oligonucleotide
cccaataggcacacttgtt

SEQ ID NO: 554 - DNA, 18 bp, Artificial Sequence
  Synthetic oligonucleotide
cctgtgtaggtcaaccac

SEQ ID NO: 555 - DNA, 19 bp, Artificial Sequence
  Synthetic oligonucleotide
accacaatgatcacacttc

SEQ ID NO: 556 - DNA, 16 bp, Artificial Sequence
  Synthetic oligonucleotide
acgtggtccagaacaa

SEQ ID NO: 557 - DNA, 18 bp, Artificial Sequence
  Synthetic oligonucleotide
aagcacacgcctattaat

SEQ ID NO: 558 - DNA, 21 bp, Artificial Sequence
  Synthetic oligonucleotide
ccttgaggaagttgtagcacg

SEQ ID NO: 559 - DNA, 20 bp, Artificial Sequence
  Synthetic oligonucleotide
cacctgtatggttacaacct

SEQ ID NO: 560 - DNA, 20 bp, Artificial Sequence
  Synthetic oligonucleotide
aagttcccctactgctgcct

SEQ ID NO: 561 - DNA, 20 bp, Artificial Sequence
  Synthetic oligonucleotide
caaattggatgacaaagatc

SEQ ID NO: 562 - DNA, 19 bp, Artificial Sequence
  Synthetic oligonucleotide
aacccaataggcacacttg

SEQ ID NO: 563 - DNA, 16 bp, Artificial Sequence
  Synthetic oligonucleotide
tttgcccccagcgctt

SEQ ID NO: 564 - DNA, 20 bp, Artificial Sequence
  Synthetic oligonucleotide
ccttgaatacaccaaaagat

SEQ ID NO: 565 - DNA, 20 bp, Artificial Sequence
  Synthetic oligonucleotide
cttgccgaataccataatga

SEQ ID NO: 566 - DNA, 19 bp, Artificial Sequence
  Synthetic oligonucleotide
acgcatacaaaacattccc

SEQ ID NO: 567 - DNA, 18 bp, Artificial Sequence
  Synthetic oligonucleotide
cacacgcctattaattta

SEQ ID NO: 568 - DNA, 19 bp, Artificial Sequence
  Synthetic oligonucleotide
aattggatgacaaagatcc

SEQ ID NO: 569 - DNA, 15 bp, Artificial Sequence
  Synthetic oligonucleotide
cagcctccaaaggca

SEQ ID NO: 570 - DNA, 18 bp, Artificial Sequence
  Synthetic oligonucleotide
aaggcgttccaattaaca

SEQ ID NO: 571 - DNA, 18 bp, Artificial Sequence
  Synthetic oligonucleotide
atctggcttgaaaaccat

SEQ ID NO: 572 - DNA, 15 bp, Artificial Sequence
  Synthetic oligonucleotide
tctgccgaaagcttg

SEQ ID NO: 573 - DNA, 17 bp, Artificial Sequence
  Synthetic oligonucleotide
acgtggaacccaatagg

SEQ ID NO: 574 - DNA, 19 bp, Artificial Sequence
  Synthetic oligonucleotide
ccattggtcccagagacat

SEQ ID NO: 575 - DNA, 20 bp, Artificial Sequence
  Synthetic oligonucleotide
tcttcaacctaggacttttc

SEQ ID NO: 576 - DNA, 18 bp, Artificial Sequence
  Synthetic oligonucleotide
ttgacgcatacaaaacat

SEQ ID NO: 577 - DNA, 15 bp, Artificial Sequence
  Synthetic oligonucleotide
cagcaccagctgtcc

SEQ ID NO: 578 - DNA, 20 bp, Artificial Sequence
  Synthetic oligonucleotide
cagtggaagcaaaataaaca

SEQ ID NO: 579 - DNA, 21 bp, Artificial Sequence
  Synthetic oligonucleotide
ccgaataccataatgaatctg

SEQ ID NO: 580 - DNA, 21 bp, Artificial Sequence
  Synthetic oligonucleotide
cccagtccctacttattgtta

SEQ ID NO: 581 - DNA, 19 bp, Artificial Sequence
  Synthetic oligonucleotide
ttgcaacccatatgatgcc

SEQ ID NO: 582 - DNA, 17 bp, Artificial Sequence
  Synthetic oligonucleotide
acaatgctgcaatcgtg

SEQ ID NO: 583 - DNA, 20 bp, Artificial Sequence
  Synthetic oligonucleotide
aaactctgaactcactttcc

SEQ ID NO: 584 - DNA, 17 bp, Artificial Sequence
  Synthetic oligonucleotide
accattggtagatttgc

SEQ ID NO: 585 - DNA, 21 bp, Artificial Sequence
  Synthetic oligonucleotide
ttctccattctggttactgcc

SEQ ID NO: 586 - DNA, 19 bp, Artificial Sequence
  Synthetic oligonucleotide
tagcaccatagggaagtcc

SEQ ID NO: 587 - DNA, 19 bp, Artificial Sequence
  Synthetic oligonucleotide
accttctttagtcaaattc

SEQ ID NO: 588 - DNA, 18 bp, Artificial Sequence
  Synthetic oligonucleotide
atgctggacaataaattt

SEQ ID NO: 589 - DNA, 16 bp, Artificial Sequence
  Synthetic oligonucleotide
ccctgagggagatcac

SEQ ID NO: 590 - DNA, 18 bp, Artificial Sequence
  Synthetic oligonucleotide
aagtagtaccaaaaatcc

SEQ ID NO: 591 - DNA, 18 bp, Artificial Sequence
  Synthetic oligonucleotide
caatcctgctaacaatgc

SEQ ID NO: 592 - DNA, 20 bp, Artificial Sequence
  Synthetic oligonucleotide
aagcctcttctcgttcctca

SEQ ID NO: 593 - DNA, 19 bp, Artificial Sequence
  Synthetic oligonucleotide
aatgtaacacaagctttcg

SEQ ID NO: 594 - DNA, 18 bp, Artificial Sequence
  Synthetic oligonucleotide
acgtggtccagaacaaac

SEQ ID NO: 595 - DNA, 20 bp, Artificial Sequence
  Synthetic oligonucleotide
ccgaagagctaccagacgaa

SEQ ID NO: 596 - DNA, 20 bp, Artificial Sequence
  Synthetic oligonucleotide
caccaaatgaatgcaaccaa

SEQ ID NO: 597 - DNA, 23 bp, Artificial Sequence
  Synthetic oligonucleotide
actgagaagtctaacataataag

SEQ ID NO: 598 - DNA, 21 bp, Artificial Sequence
  Synthetic oligonucleotide
caaggaactgattacaaacat

SEQ ID NO: 599 - DNA, 16 bp, Artificial Sequence
  Synthetic oligonucleotide
ttctcccgcactcttg

SEQ ID NO: 600 - DNA, 17 bp, Artificial Sequence
  Synthetic oligonucleotide
caagctggttcaatctg

SEQ ID NO: 601 - DNA, 20 bp, Artificial Sequence
  Synthetic oligonucleotide
ctcatgaagtgtgatcattg

SEQ ID NO: 602 - DNA, 23 bp, Artificial Sequence
  Synthetic oligonucleotide
ccaggagtcaaataacttctatg

SEQ ID NO: 603 - DNA, 19 bp, Artificial Sequence
  Synthetic oligonucleotide
atgcttattcagcaaaatg

SEQ ID NO: 604 - DNA, 18 bp, Artificial Sequence
  Synthetic oligonucleotide
tcacgtagtcgcaacagt

SEQ ID NO: 605 - DNA, 23 bp, Artificial Sequence
  Synthetic oligonucleotide
tagtcttgattatggaatttaag

SEQ ID NO: 606 - DNA, 21 bp, Artificial Sequence
  Synthetic oligonucleotide
cactttccatccaacttttgt

SEQ ID NO: 607 - DNA, 17 bp, Artificial Sequence
  Synthetic oligonucleotide
tcattgtggtgaaactt

SEQ ID NO: 608 - DNA, 19 bp, Artificial Sequence
  Synthetic oligonucleotide
ttggtcccagagacatgta

SEQ ID NO: 609 - DNA, 19 bp, Artificial Sequence
  Synthetic oligonucleotide
caatagcagtccagatgac

SEQ ID NO: 610 - DNA, 19 bp, Artificial Sequence
  Synthetic oligonucleotide
ttggtggaccctcagattc

SEQ ID NO: 611 - DNA, 23 bp, Artificial Sequence
  Synthetic oligonucleotide
caaacattgttcgtttagttgtt

SEQ ID NO: 612 - DNA, 20 bp, Artificial Sequence
  Synthetic oligonucleotide
tcatcagccttcttcttttt

SEQ ID NO: 613 - DNA, 19 bp, Artificial Sequence
  Synthetic oligonucleotide
ccaaacaattgcaacaatc

SEQ ID NO: 614 - DNA, 21 bp, Artificial Sequence
  Synthetic oligonucleotide
ccatttttgggtgtttattac

SEQ ID NO: 615 - DNA, 22 bp, Artificial Sequence
  Synthetic oligonucleotide
ccagttcctaggtagtagaaat

SEQ ID NO: 616 - DNA, 19 bp, Artificial Sequence
  Synthetic oligonucleotide
attcgtctggtagctcttc

SEQ ID NO: 617 - DNA, 20 bp, Artificial Sequence
  Synthetic oligonucleotide
caaagatcaagtcattttgc

SEQ ID NO: 618 - DNA, 18 bp, Artificial Sequence
  Synthetic oligonucleotide
ctggtaaaggccaacaac

SEQ ID NO: 619 - DNA, 18 bp, Artificial Sequence
  Synthetic oligonucleotide
accccctgcatacactaa

SEQ ID NO: 620 - DNA, 19 bp, Artificial Sequence
  Synthetic oligonucleotide
ccttctttagtcaaattct

SEQ ID NO: 621 - DNA, 16 bp, Artificial Sequence
  Synthetic oligonucleotide
ctggagttgaatttct

SEQ ID NO: 622 - DNA, 18 bp, Artificial Sequence
  Synthetic oligonucleotide
caaaatcgcccgtctgcc

SEQ ID NO: 623 - DNA, 17 bp, Artificial Sequence
  Synthetic oligonucleotide
caacaaggccaaactgt

SEQ ID NO: 624 - DNA, 17 bp, Artificial Sequence
  Synthetic oligonucleotide
ttggatgacaaagatcc

SEQ ID NO: 625 - DNA, 19 bp, Artificial Sequence
  Synthetic oligonucleotide
aatgaatgcaaccaaatgt

SEQ ID NO: 626 - DNA, 18 bp, Artificial Sequence
  Synthetic oligonucleotide
caaaatttggacattccc

SEQ ID NO: 627 - DNA, 19 bp, Artificial Sequence
  Synthetic oligonucleotide
ctgtaatggttccattttc

SEQ ID NO: 628 - DNA, 19 bp, Artificial Sequence
  Synthetic oligonucleotide
aagcctcttctcgttcctc

SEQ ID NO: 629 - DNA, 17 bp, Artificial Sequence
  Synthetic oligonucleotide
ccagcatgtcacaattc

SEQ ID NO: 630 - DNA, 21 bp, Artificial Sequence
  Synthetic oligonucleotide
cagttccttgtctgattagtt

SEQ ID NO: 631 - DNA, 18 bp, Artificial Sequence
  Synthetic oligonucleotide
ccgtcaccaccacgaatt

SEQ ID NO: 632 - DNA, 17 bp, Artificial Sequence
  Synthetic oligonucleotide
ccctgacaaagttttca

SEQ ID NO: 633 - DNA, 19 bp, Artificial Sequence
  Synthetic oligonucleotide
aaagtgtacgttgaaatcc

SEQ ID NO: 634 - DNA, 17 bp, Artificial Sequence
  Synthetic oligonucleotide
ctgagggtccaccaaac

SEQ ID NO: 635 - DNA, 19 bp, Artificial Sequence
  Synthetic oligonucleotide
tcaaaagtgcaattattcg

SEQ ID NO: 636 - DNA, 20 bp, Artificial Sequence
  Synthetic oligonucleotide
ctctagtcagtgtgttaatc

SEQ ID NO: 637 - DNA, 14 bp, Artificial Sequence
  Synthetic oligonucleotide
ctgccgaaagcttg

SEQ ID NO: 638 - DNA, 17 bp, Artificial Sequence
  Synthetic oligonucleotide
atcgcccgtctgccatg

SEQ ID NO: 639 - DNA, 18 bp, Artificial Sequence
  Synthetic oligonucleotide
aagaggcttgactgccgc

SEQ ID NO: 640 - DNA, 17 bp, Artificial Sequence
  Synthetic oligonucleotide
cctctgctcccttctgc

SEQ ID NO: 641 - DNA, 19 bp, Artificial Sequence
  Synthetic oligonucleotide
atggcaaccaacataagag

SEQ ID NO: 642 - DNA, 25 bp, Artificial Sequence
  Synthetic oligonucleotide
atggttccattttcattatatttta

SEQ ID NO: 643 - DNA, 17 bp, Artificial Sequence
  Synthetic oligonucleotide
ctgcgttctccattctg

SEQ ID NO: 644 - DNA, 20 bp, Artificial Sequence
  Synthetic oligonucleotide
ccaggagtcaaataacttct

SEQ ID NO: 645 - DNA, 19 bp, Artificial Sequence
  Synthetic oligonucleotide
caaaatttggacattcccc

SEQ ID NO: 646 - DNA, 18 bp, Artificial Sequence
  Synthetic oligonucleotide
tactgctgcctggagttg

SEQ ID NO: 647 - DNA, 25 bp, Artificial Sequence
  Synthetic oligonucleotide
ccatcaatattcttaaacacaaatt

SEQ ID NO: 648 - DNA, 20 bp, Artificial Sequence
  Synthetic oligonucleotide
aagagctaccagacgaattc

SEQ ID NO: 649 - DNA, 19 bp, Artificial Sequence
  Synthetic oligonucleotide
cctgtcctaccatttaatg

SEQ ID NO: 650 - DNA, 18 bp, Artificial Sequence
  Synthetic oligonucleotide
ccctgcatacactaattc

SEQ ID NO: 651 - DNA, 16 bp, Artificial Sequence
  Synthetic oligonucleotide
ctgccgaaagcttgtg

SEQ ID NO: 652 - DNA, 20 bp, Artificial Sequence
  Synthetic oligonucleotide
tcatgagagttgaaaggcac

SEQ ID NO: 653 - DNA, 22 bp, Artificial Sequence
  Synthetic oligonucleotide
ctgtgactcttcttcctgctgc

SEQ ID NO: 654 - DNA, 16 bp, Artificial Sequence
  Synthetic oligonucleotide
catggcaaggaagacc

SEQ ID NO: 655 - DNA, 18 bp, Artificial Sequence
  Synthetic oligonucleotide
cctgagcatagtcttgcc

SEQ ID NO: 656 - DNA, 16 bp, Artificial Sequence
  Synthetic oligonucleotide
tcaccaccacgaattc

SEQ ID NO: 657 - DNA, 19 bp, Artificial Sequence
  Synthetic oligonucleotide
aagaaggctgatgaaactc

SEQ ID NO: 658 - DNA, 16 bp, Artificial Sequence
  Synthetic oligonucleotide
ttccatgccaatgcgc

SEQ ID NO: 659 - DNA, 19 bp, Artificial Sequence
  Synthetic oligonucleotide
aagtggctttaacaaaatc

SEQ ID NO: 660 - DNA, 19 bp, Artificial Sequence
  Synthetic oligonucleotide
ttcggtagtagccaatttg

SEQ ID NO: 661 - DNA, 18 bp, Artificial Sequence
  Synthetic oligonucleotide
aagccatcaagctttttc

SEQ ID NO: 662 - DNA, 18 bp, Artificial Sequence
  Synthetic oligonucleotide
ctgcgttctccattctgg

SEQ ID NO: 663 - DNA, 19 bp, Artificial Sequence
  Synthetic oligonucleotide
ttggaacgccttgtcctcg

SEQ ID NO: 664 - DNA, 20 bp, Artificial Sequence
  Synthetic oligonucleotide
ttggacattccccattgaag

SEQ ID NO: 665 - DNA, 21 bp, Artificial Sequence
  Synthetic oligonucleotide
ctcgttcctcatcacgtagtc

SEQ ID NO: 666 - DNA, 17 bp, Artificial Sequence
  Synthetic oligonucleotide
ctgggtcttcgaatcta

SEQ ID NO: 667 - DNA, 19 bp, Artificial Sequence
  Synthetic oligonucleotide
tctacgcagaagggagcag

SEQ ID NO: 668 - DNA, 17 bp, Artificial Sequence
  Synthetic oligonucleotide
attggcatggaagtcac

SEQ ID NO: 669 - DNA, 21 bp, Artificial Sequence
  Synthetic oligonucleotide
tcagcaaaatgacttgatctt

SEQ ID NO: 670 - DNA, 19 bp, Artificial Sequence
  Synthetic oligonucleotide
cttcaacctaggacttttc

SEQ ID NO: 671 - DNA, 16 bp, Artificial Sequence
  Synthetic oligonucleotide
ctcctggtgattcttc

SEQ ID NO: 672 - DNA, 18 bp, Artificial Sequence
  Synthetic oligonucleotide
ttctagtgcgaataattg

SEQ ID NO: 673 - DNA, 18 bp, Artificial Sequence
  Synthetic oligonucleotide
ccgacgttgttttgatcg

SEQ ID NO: 674 - DNA, 17 bp, Artificial Sequence
  Synthetic oligonucleotide
cttgagagcaaaatgtc

SEQ ID NO: 675 - DNA, 18 bp, Artificial Sequence
  Synthetic oligonucleotide
ctctgggaccaatggtac

SEQ ID NO: 676 - DNA, 21 bp, Artificial Sequence
  Synthetic oligonucleotide
tctcatgaagtgtgatcattg

SEQ ID NO: 677 - DNA, 17 bp, Artificial Sequence
  Synthetic oligonucleotide
aaggcgttccaattaac

SEQ ID NO: 678 - DNA, 17 bp, Artificial Sequence
  Synthetic oligonucleotide
cagcagcagatttctta

SEQ ID NO: 679 - DNA, 17 bp, Artificial Sequence
  Synthetic oligonucleotide
caggtgccatcaaattg

SEQ ID NO: 680 - DNA, 18 bp, Artificial Sequence
  Synthetic oligonucleotide
aagggtggtcgcactatt

SEQ ID NO: 681 - DNA, 16 bp, Artificial Sequence
  Synthetic oligonucleotide
atcaccgccattgcca

SEQ ID NO: 682 - DNA, 15 bp, Artificial Sequence
  Synthetic oligonucleotide
cagcagcaaagcaag

SEQ ID NO: 683 - DNA, 17 bp, Artificial Sequence
  Synthetic oligonucleotide
cctattggcaaatctac

SEQ ID NO: 684 - DNA, 19 bp, Artificial Sequence
  Synthetic oligonucleotide
accagaactcaattacccc

SEQ ID NO: 685 - DNA, 20 bp, Artificial Sequence
  Synthetic oligonucleotide
catcactaggtttcaaactt

SEQ ID NO: 686 - DNA, 18 bp, Artificial Sequence
  Synthetic oligonucleotide
ccaacagagcctaaaaag

SEQ ID NO: 687 - DNA, 20 bp, Artificial Sequence
  Synthetic oligonucleotide
cattgtggtgaaacttcatg

SEQ ID NO: 688 - DNA, 22 bp, Artificial Sequence
  Synthetic oligonucleotide
aatattcttaaacacaaattcc

SEQ ID NO: 689 - DNA, 21 bp, Artificial Sequence
  Synthetic oligonucleotide
ccaatggtactaagaggtttg

SEQ ID NO: 690 - DNA, 20 bp, Artificial Sequence
  Synthetic oligonucleotide
actgccactaaagcatacaa

SEQ ID NO: 691 - DNA, 19 bp, Artificial Sequence
  Synthetic oligonucleotide
catagtcttgccgaatacc

SEQ ID NO: 692 - DNA, 18 bp, Artificial Sequence
  Synthetic oligonucleotide
acacaggtgccatcaaat

SEQ ID NO: 693 - DNA, 19 bp, Artificial Sequence
  Synthetic oligonucleotide
ctgggtcttcgaatctaaa

SEQ ID NO: 694 - DNA, 20 bp, Artificial Sequence
  Synthetic oligonucleotide
cattcaactcaggacttgtt

SEQ ID NO: 695 - DNA, 17 bp, Artificial Sequence
  Synthetic oligonucleotide
ccacgttcccgaaggtg

SEQ ID NO: 696 - DNA, 16 bp, Artificial Sequence
  Synthetic oligonucleotide
ccaatgcgcgacattc

SEQ ID NO: 697 - DNA, 20 bp, Artificial Sequence
  Synthetic oligonucleotide
acagatgctgtagactgtgc

SEQ ID NO: 698 - DNA, 19 bp, Artificial Sequence
  Synthetic oligonucleotide
aataagaggctggattttt

SEQ ID NO: 699 - DNA, 19 bp, Artificial Sequence
  Synthetic oligonucleotide
atggatcattacaaaattg

SEQ ID NO: 700 - DNA, 20 bp, Artificial Sequence
  Synthetic oligonucleotide
ccttacgaagaatggttttc

SEQ ID NO: 701 - DNA, 20 bp, Artificial Sequence
  Synthetic oligonucleotide
actcaggacttgttcttacc

SEQ ID NO: 702 - DNA, 17 bp, Artificial Sequence
  Synthetic oligonucleotide
aagcagcagcaaagcaa

SEQ ID NO: 703 - DNA, 18 bp, Artificial Sequence
  Synthetic oligonucleotide
acaaacccaaggaaattt

SEQ ID NO: 704 - DNA, 18 bp, Artificial Sequence
  Synthetic oligonucleotide
ccaatggttctaaagccg

SEQ ID NO: 705 - DNA, 20 bp, Artificial Sequence
  Synthetic oligonucleotide
acaatgtaacacaagctttc

SEQ ID NO: 706 - DNA, 17 bp, Artificial Sequence
  Synthetic oligonucleotide
caccgctctcactcaac

SEQ ID NO: 707 - DNA, 18 bp, Artificial Sequence
  Synthetic oligonucleotide
cccagtccctacttattg

SEQ ID NO: 708 - DNA, 14 bp, Artificial Sequence
  Synthetic oligonucleotide
ttgcggccaatgtt

SEQ ID NO: 709 - DNA, 17 bp, Artificial Sequence
  Synthetic oligonucleotide
ttgcggccaatgtttgt

SEQ ID NO: 710 - DNA, 18 bp, Artificial Sequence
  Synthetic oligonucleotide
tcagtggaagcaaaataa

SEQ ID NO: 711 - DNA, 21 bp, Artificial Sequence
  Synthetic oligonucleotide
cccttggttgaatagtcttga

SEQ ID NO: 712 - DNA, 17 bp, Artificial Sequence
  Synthetic oligonucleotide
cccaccaacagagccta

SEQ ID NO: 713 - DNA, 17 bp, Artificial Sequence
  Synthetic oligonucleotide
cacccttacgaagaatg

SEQ ID NO: 714 - DNA, 18 bp, Artificial Sequence
  Synthetic oligonucleotide
tcgtgctacaacttcctc

SEQ ID NO: 715 - DNA, 17 bp, Artificial Sequence
  Synthetic oligonucleotide
ctgcagcaccagctgtc

SEQ ID NO: 716 - DNA, 16 bp, Artificial Sequence
  Synthetic oligonucleotide
ctagcacgtggaaccc

SEQ ID NO: 717 - DNA, 18 bp, Artificial Sequence
  Synthetic oligonucleotide
cacacgtggtgtttatta

SEQ ID NO: 718 - DNA, 19 bp, Artificial Sequence
  Synthetic oligonucleotide
ctgagggagccttgaatac

SEQ ID NO: 719 - DNA, 19 bp, Artificial Sequence
  Synthetic oligonucleotide
ttcttaaacacaaattccc

SEQ ID NO: 720 - DNA, 16 bp, Artificial Sequence
  Synthetic oligonucleotide
cagcatgtcacaattc

SEQ ID NO: 721 - DNA, 19 bp, Artificial Sequence
  Synthetic oligonucleotide
cagggtttttcggctttag

SEQ ID NO: 722 - DNA, 17 bp, Artificial Sequence
  Synthetic oligonucleotide
accaggagtcaaataac

SEQ ID NO: 723 - DNA, 19 bp, Artificial Sequence
  Synthetic oligonucleotide
ccatatgatgccgtctttg

SEQ ID NO: 724 - DNA, 15 bp, Artificial Sequence
  Synthetic oligonucleotide
ccccagcgcttcagc

SEQ ID NO: 725 - DNA, 18 bp, Artificial Sequence
  Synthetic oligonucleotide
aatgtgcctttcaactct

SEQ ID NO: 726 - DNA, 20 bp, Artificial Sequence
  Synthetic oligonucleotide
aagatcacattggcacccgc

SEQ ID NO: 727 - DNA, 20 bp, Artificial Sequence
  Synthetic oligonucleotide
ccttgtctgattagttcctg

SEQ ID NO: 728 - DNA, 18 bp, Artificial Sequence
  Synthetic oligonucleotide
tcgcactagaataaactc

SEQ ID NO: 729 - DNA, 18 bp, Artificial Sequence
  Synthetic oligonucleotide
atgtctctcagccttttc

SEQ ID NO: 730 - DNA, 23 bp, Artificial Sequence
  Synthetic oligonucleotide
acaagaaaaacaaacattgttcg

SEQ ID NO: 731 - DNA, 22 bp, Artificial Sequence
  Synthetic oligonucleotide
ttaatgatggtgtttattttgc

SEQ ID NO: 732 - DNA, 18 bp, Artificial Sequence
  Synthetic oligonucleotide
cctctctcagaaacaaag

SEQ ID NO: 733 - DNA, 19 bp, Artificial Sequence
  Synthetic oligonucleotide
cagcaaactgtgactcttc

SEQ ID NO: 734 - DNA, 16 bp, Artificial Sequence
  Synthetic oligonucleotide
atcgcgccccactgcg

SEQ ID NO: 735 - DNA, 18 bp, Artificial Sequence
  Synthetic oligonucleotide
cacgtagtcgcaacagtt

SEQ ID NO: 736 - DNA, 19 bp, Artificial Sequence
  Synthetic oligonucleotide
ttggccgcaaattgcacaa

SEQ ID NO: 737 - DNA, 20 bp, Artificial Sequence
  Synthetic oligonucleotide
ccttctttagtcaaattctc

SEQ ID NO: 738 - DNA, 18 bp, Artificial Sequence
  Synthetic oligonucleotide
tcggtagtagccaatttg

SEQ ID NO: 739 - DNA, 16 bp, Artificial Sequence
  Synthetic oligonucleotide
ctgccagttgaatctg

SEQ ID NO: 740 - DNA, 16 bp, Artificial Sequence
  Synthetic oligonucleotide
ttttgccgaggcttct

SEQ ID NO: 741 - DNA, 17 bp, Artificial Sequence
  Synthetic oligonucleotide
ctgctgaggcttctaag

SEQ ID NO: 742 - DNA, 16 bp, Artificial Sequence
  Synthetic oligonucleotide
aaggctgatgaaactc

SEQ ID NO: 743 - DNA, 16 bp, Artificial Sequence
  Synthetic oligonucleotide
atttgcggccaatgtt

SEQ ID NO: 744 - DNA, 21 bp, Artificial Sequence
  Synthetic oligonucleotide
atccatttttgggtgtttatt

SEQ ID NO: 745 - DNA, 18 bp, Artificial Sequence
  Synthetic oligonucleotide
aacatggcaaggaagacc

SEQ ID NO: 746 - DNA, 24 bp, Artificial Sequence
  Synthetic oligonucleotide
cctaggacttttctattaaaatat

SEQ ID NO: 747 - DNA, 20 bp, Artificial Sequence
  Synthetic oligonucleotide
caacgtacactttgtttctg

SEQ ID NO: 748 - DNA, 20 bp, Artificial Sequence
  Synthetic oligonucleotide
catgtatagcatggaaccaa

SEQ ID NO: 749 - DNA, 20 bp, Artificial Sequence
  Synthetic oligonucleotide
atcactaggtttcaaacttt

SEQ ID NO: 750 - DNA, 17 bp, Artificial Sequence
  Synthetic oligonucleotide
acatgctggacaataaa

SEQ ID NO: 751 - DNA, 18 bp, Artificial Sequence
  Synthetic oligonucleotide
ctgccagttgaatctgag

SEQ ID NO: 752 - DNA, 21 bp, Artificial Sequence
  Synthetic oligonucleotide
actctcatgaagtgtgatcat

SEQ ID NO: 753 - DNA, 22 bp, Artificial Sequence
  Synthetic oligonucleotide
aaagtctgtgaatttcaatttt

SEQ ID NO: 754 - DNA, 15 bp, Artificial Sequence
  Synthetic oligonucleotide
tcagcagcagatttc

SEQ ID NO: 755 - DNA, 15 bp, Artificial Sequence
  Synthetic oligonucleotide
ctgcggtaaggcttg

SEQ ID NO: 756 - DNA, 17 bp, Artificial Sequence
  Synthetic oligonucleotide
cattggcacccgcaatc

SEQ ID NO: 757 - DNA, 16 bp, Artificial Sequence
  Synthetic oligonucleotide
ccgaaggtgtgacttc

SEQ ID NO: 758 - DNA, 19 bp, Artificial Sequence
  Synthetic oligonucleotide
attaccccctgcatacact

SEQ ID NO: 759 - DNA, 20 bp, Artificial Sequence
  Synthetic oligonucleotide
tcgtggtggtgacggtaaaa

SEQ ID NO: 760 - DNA, 14 bp, Artificial Sequence
  Synthetic oligonucleotide
ttggccgcaaattg

SEQ ID NO: 761 - DNA, 25 bp, Artificial Sequence
  Synthetic oligonucleotide
catcaatattcttaaacacaaattc

SEQ ID NO: 762 - DNA, 16 bp, Artificial Sequence
  Synthetic oligonucleotide
ctcagtgccacaaaat

SEQ ID NO: 763 - DNA, 18 bp, Artificial Sequence
  Synthetic oligonucleotide
ccaagacgcagtattatt

SEQ ID NO: 764 - DNA, 17 bp, Artificial Sequence
  Synthetic oligonucleotide
caagagcagcatcaccg

SEQ ID NO: 765 - DNA, 18 bp, Artificial Sequence
  Synthetic oligonucleotide
accacgtgtgaaagaatt

SEQ ID NO: 766 - DNA, 19 bp, Artificial Sequence
  Synthetic oligonucleotide
actttgtcagggtaataaa

SEQ ID NO: 767 - DNA, 17 bp, Artificial Sequence
  Synthetic oligonucleotide
ttgcgggtgccaatgtg

SEQ ID NO: 768 - DNA, 17 bp, Artificial Sequence
  Synthetic oligonucleotide
aaggctgatgaaactca

SEQ ID NO: 769 - DNA, 19 bp, Artificial Sequence
  Synthetic oligonucleotide
cagatttggatgatttctc

SEQ ID NO: 770 - DNA, 18 bp, Artificial Sequence
  Synthetic oligonucleotide
cagcatgtcacaattcag

SEQ ID NO: 771 - DNA, 19 bp, Artificial Sequence
  Synthetic oligonucleotide
cctacacaggtgccatcaa

SEQ ID NO: 772 - DNA, 15 bp, Artificial Sequence
  Synthetic oligonucleotide
tcggccccaaggttt

SEQ ID NO: 773 - DNA, 18 bp, Artificial Sequence
  Synthetic oligonucleotide
aaccagaactcaattacc

SEQ ID NO: 774 - DNA, 14 bp, Artificial Sequence
  Synthetic oligonucleotide
atgcgcgacattcc

SEQ ID NO: 775 - DNA, 20 bp, Artificial Sequence
  Synthetic oligonucleotide
tagagactagtggcaataaa

SEQ ID NO: 776 - DNA, 21 bp, Artificial Sequence
  Synthetic oligonucleotide
caggacttgttcttacctttc

SEQ ID NO: 777 - DNA, 17 bp, Artificial Sequence
  Synthetic oligonucleotide
cacttgcgaattttgtg

SEQ ID NO: 778 - DNA, 21 bp, Artificial Sequence
  Synthetic oligonucleotide
aagtgagttcagagtttattc

SEQ ID NO: 779 - DNA, 19 bp, Artificial Sequence
  Synthetic oligonucleotide
aagagaacacacagcctcc

SEQ ID NO: 780 - DNA, 15 bp, Artificial Sequence
  Synthetic oligonucleotide
cacttgcgaattttg

SEQ ID NO: 781 - DNA, 24 bp, Artificial Sequence
  Synthetic oligonucleotide
atggttacaacctatgttagcgct

SEQ ID NO: 782 - DNA, 16 bp, Artificial Sequence
  Synthetic oligonucleotide
ctgccgcctctgctcc

SEQ ID NO: 783 - DNA, 16 bp, Artificial Sequence
  Synthetic oligonucleotide
tcggccccaaggttta

SEQ ID NO: 784 - DNA, 21 bp, Artificial Sequence
  Synthetic oligonucleotide
ttttcagatcctcagttttac

SEQ ID NO: 785 - DNA, 19 bp, Artificial Sequence
  Synthetic oligonucleotide
ttggtgtattcaaggctcc

SEQ ID NO: 786 - DNA, 16 bp, Artificial Sequence
  Synthetic oligonucleotide
tctctgcggtaaggct

SEQ ID NO: 787 - DNA, 19 bp, Artificial Sequence
  Synthetic oligonucleotide
accaaatgtgcctttcaac

SEQ ID NO: 788 - DNA, 17 bp, Artificial Sequence
  Synthetic oligonucleotide
cagttgcgtcaccaaat

SEQ ID NO: 789 - DNA, 17 bp, Artificial Sequence
  Synthetic oligonucleotide
acaggtgccatcaaatt

SEQ ID NO: 790 - DNA, 20 bp, Artificial Sequence
  Synthetic oligonucleotide
ttgtatttcccttaaattcc

SEQ ID NO: 791 - DNA, 18 bp, Artificial Sequence
  Synthetic oligonucleotide
acaagtagtggcaccttc

SEQ ID NO: 792 - DNA, 19 bp, Artificial Sequence
  Synthetic oligonucleotide
ccaggagtcaaataacttc

SEQ ID NO: 793 - DNA, 21 bp, Artificial Sequence
  Synthetic oligonucleotide
atcgaattctacccataaagc

SEQ ID NO: 794 - DNA, 23 bp, Artificial Sequence
  Synthetic oligonucleotide
ttgaaacctagtgatgttaatac

SEQ ID NO: 795 - DNA, 16 bp, Artificial Sequence
  Synthetic oligonucleotide
aacccaaggaaatttt

SEQ ID NO: 796 - DNA, 22 bp, Artificial Sequence
  Synthetic oligonucleotide
acatattcaaaagtgcaattat

SEQ ID NO: 797 - DNA, 21 bp, Artificial Sequence
  Synthetic oligonucleotide
caggtcctacttctgaattgt

SEQ ID NO: 798 - DNA, 20 bp, Artificial Sequence
  Synthetic oligonucleotide
ttgccgaataccataatgaa

SEQ ID NO: 799 - DNA, 17 bp, Artificial Sequence
  Synthetic oligonucleotide
ctccaaaggcaatagtg

SEQ ID NO: 800 - DNA, 19 bp, Artificial Sequence
  Synthetic oligonucleotide
ccaaggtttacccaataat

SEQ ID NO: 801 - DNA, 18 bp, Artificial Sequence
  Synthetic oligonucleotide
ttggaacgccttgtcctc

SEQ ID NO: 802 - DNA, 16 bp, Artificial Sequence
  Synthetic oligonucleotide
ttggcacccgcaatcc

SEQ ID NO: 803 - DNA, 19 bp, Artificial Sequence
  Synthetic oligonucleotide
caacaaggccaaactgtca

SEQ ID NO: 804 - DNA, 19 bp, Artificial Sequence
  Synthetic oligonucleotide
caaggaagaccttaaattc

SEQ ID NO: 805 - DNA, 18 bp, Artificial Sequence
  Synthetic oligonucleotide
tcgcaagtggctttaaca

SEQ ID NO: 806 - DNA, 20 bp, Artificial Sequence
  Synthetic oligonucleotide
acggtaaaatgaaagatctc

SEQ ID NO: 807 - DNA, 16 bp, Artificial Sequence
  Synthetic oligonucleotide
ccattgccagccattc

SEQ ID NO: 808 - DNA, 21 bp, Artificial Sequence
  Synthetic oligonucleotide
ccctaagatttttgaaattac

SEQ ID NO: 809 - DNA, 19 bp, Artificial Sequence
  Synthetic oligonucleotide
ctggtaaaggccaacaaca

SEQ ID NO: 810 - DNA, 20 bp, Artificial Sequence
  Synthetic oligonucleotide
ttcaaggtccataagaaaag

SEQ ID NO: 811 - DNA, 18 bp, Artificial Sequence
  Synthetic oligonucleotide
ctgctcatggattgttgc

SEQ ID NO: 812 - DNA, 23 bp, Artificial Sequence
  Synthetic oligonucleotide
cagccttcttctttttgtccttt

SEQ ID NO: 813 - DNA, 16 bp, Artificial Sequence
  Synthetic oligonucleotide
ctgctgcctggagttg

SEQ ID NO: 814 - DNA, 16 bp, Artificial Sequence
  Synthetic oligonucleotide
cctcagcagcagattt

SEQ ID NO: 815 - DNA, 20 bp, Artificial Sequence
  Synthetic oligonucleotide
ctagtcagtgtgttaatctt

SEQ ID NO: 816 - DNA, 19 bp, Artificial Sequence
  Synthetic oligonucleotide
tctggcttgaaaaccattc

SEQ ID NO: 817 - DNA, 20 bp, Artificial Sequence
  Synthetic oligonucleotide
cttccactgagaagtctaac

SEQ ID NO: 818 - DNA, 17 bp, Artificial Sequence
  Synthetic oligonucleotide
ttagtgcgtgatctccc

SEQ ID NO: 819 - DNA, 22 bp, Artificial Sequence
  Synthetic oligonucleotide
ttgagttctggttgtaagatta

SEQ ID NO: 820 - DNA, 18 bp, Artificial Sequence
  Synthetic oligonucleotide
tcttaaacacaaattccc

SEQ ID NO: 821 - DNA, 19 bp, Artificial Sequence
  Synthetic oligonucleotide
tttgatcgcgccccactgc

SEQ ID NO: 822 - DNA, 24 bp, Artificial Sequence
  Synthetic oligonucleotide
ttgttattaaagtctgtgaatttc

SEQ ID NO: 823 - DNA, 18 bp, Artificial Sequence
  Synthetic oligonucleotide
ctgtccaacctgaagaag

SEQ ID NO: 824 - DNA, 17 bp, Artificial Sequence
  Synthetic oligonucleotide
aaaacaacgtcggcccc

SEQ ID NO: 825 - DNA, 20 bp, Artificial Sequence
  Synthetic oligonucleotide
cagcaggaagaagagtcaca

SEQ ID NO: 826 - DNA, 20 bp, Artificial Sequence
  Synthetic oligonucleotide
atgctcaggtcctacttctg

SEQ ID NO: 827 - DNA, 17 bp, Artificial Sequence
  Synthetic oligonucleotide
aatctggcttgaaaacc

SEQ ID NO: 828 - DNA, 19 bp, Artificial Sequence
  Synthetic oligonucleotide
ctgcaatcgtgctacaact

SEQ ID NO: 829 - DNA, 17 bp, Artificial Sequence
  Synthetic oligonucleotide
tcgtctggtagctcttc

SEQ ID NO: 830 - DNA, 21 bp, Artificial Sequence
  Synthetic oligonucleotide
tcttagaagcctcagcagcag

SEQ ID NO: 831 - DNA, 18 bp, Artificial Sequence
  Synthetic oligonucleotide
acgtactgccactaaagc

SEQ ID NO: 832 - DNA, 21 bp, Artificial Sequence
  Synthetic oligonucleotide
ccctgtcctaccatttaatga

SEQ ID NO: 833 - DNA, 25 bp, Artificial Sequence
  Synthetic oligonucleotide
tcttgctttgctgctgcttgacaga

SEQ ID NO: 834 - DNA, 19 bp, Artificial Sequence
  Synthetic oligonucleotide
cacgtggtgtttattaccc

SEQ ID NO: 835 - DNA, 19 bp, Artificial Sequence
  Synthetic oligonucleotide
cagcatctgtaatggttcc

SEQ ID NO: 836 - DNA, 16 bp, Artificial Sequence
  Synthetic oligonucleotide
tagtgcgtgatctccc

SEQ ID NO: 837 - DNA, 20 bp, Artificial Sequence
  Synthetic oligonucleotide
ttcctaggtagtagaaatac

SEQ ID NO: 838 - DNA, 19 bp, Artificial Sequence
  Synthetic oligonucleotide
ctctcaagctggttcaatc

SEQ ID NO: 839 - DNA, 18 bp, Artificial Sequence
  Synthetic oligonucleotide
tagcaggagaagttcccc

SEQ ID NO: 840 - DNA, 19 bp, Artificial Sequence
  Synthetic oligonucleotide
aaccagaatggagaacgca

SEQ ID NO: 841 - DNA, 22 bp, Artificial Sequence
  Synthetic oligonucleotide
acataataagaggctggatttt

SEQ ID NO: 842 - DNA, 18 bp, Artificial Sequence
  Synthetic oligonucleotide
acgtagtcgcaacagttc

SEQ ID NO: 843 - DNA, 18 bp, Artificial Sequence
  Synthetic oligonucleotide
cactgctcatggattgtt

SEQ ID NO: 844 - DNA, 25 bp, Artificial Sequence
  Synthetic oligonucleotide
attgttaataacgctactaatgttg

SEQ ID NO: 845 - DNA, 17 bp, Artificial Sequence
  Synthetic oligonucleotide
tcgcaagtggctttaac

SEQ ID NO: 846 - DNA, 19 bp, Artificial Sequence
  Synthetic oligonucleotide
ctgctattggtgttaattg

SEQ ID NO: 847 - DNA, 20 bp, Artificial Sequence
  Synthetic oligonucleotide
aaggctgagagacatattca

SEQ ID NO: 848 - DNA, 24 bp, Artificial Sequence
  Synthetic oligonucleotide
atggttccattttcattatatttt

SEQ ID NO: 849 - DNA, 19 bp, Artificial Sequence
  Synthetic oligonucleotide
acttgatctttgaaatttg

SEQ ID NO: 850 - DNA, 19 bp, Artificial Sequence
  Synthetic oligonucleotide
tcactttccatccaacttt

SEQ ID NO: 851 - DNA, 18 bp, Artificial Sequence
  Synthetic oligonucleotide
attcccaccaacagagcc

SEQ ID NO: 852 - DNA, 16 bp, Artificial Sequence
  Synthetic oligonucleotide
tcgtgctacaacttcc

SEQ ID NO: 853 - DNA, 15 bp, Artificial Sequence
  Synthetic oligonucleotide
cctgctgcagatttg

SEQ ID NO: 854 - DNA, 22 bp, Artificial Sequence
  Synthetic oligonucleotide
tcctttttaggctctgttggtg

SEQ ID NO: 855 - DNA, 20 bp, Artificial Sequence
  Synthetic oligonucleotide
caaactgtgactcttcttcc

SEQ ID NO: 856 - DNA, 18 bp, Artificial Sequence
  Synthetic oligonucleotide
atgctgctcttgctttgc

SEQ ID NO: 857 - DNA, 18 bp, Artificial Sequence
  Synthetic oligonucleotide
acctattggcaaatctac

SEQ ID NO: 858 - DNA, 20 bp, Artificial Sequence
  Synthetic oligonucleotide
ctgccactaaagcatacaat

SEQ ID NO: 859 - DNA, 16 bp, Artificial Sequence
  Synthetic oligonucleotide
ctggaccacgtctgcc

SEQ ID NO: 860 - DNA, 18 bp, Artificial Sequence
  Synthetic oligonucleotide
aaggccaacaacaacaag

SEQ ID NO: 861 - DNA, 17 bp, Artificial Sequence
  Synthetic oligonucleotide
catggcaaggaagacct

SEQ ID NO: 862 - DNA, 18 bp, Artificial Sequence
  Synthetic oligonucleotide
ccaacctgaagaagaatc

SEQ ID NO: 863 - DNA, 15 bp, Artificial Sequence
  Synthetic oligonucleotide
ccccactgcgttctc

SEQ ID NO: 864 - DNA, 28 bp, Artificial Sequence
  Synthetic oligonucleotide
tcccagagacatgtatagcatggaacca

SEQ ID NO: 865 - DNA, 20 bp, Artificial Sequence
  Synthetic oligonucleotide
atagcatggaaccaagtaac

SEQ ID NO: 866 - DNA, 19 bp, Artificial Sequence
  Synthetic oligonucleotide
ctgcggtaaggcttgagtt

SEQ ID NO: 867 - DNA, 20 bp, Artificial Sequence
  Synthetic oligonucleotide
taataagaggctggattttt

SEQ ID NO: 868 - DNA, 18 bp, Artificial Sequence
  Synthetic oligonucleotide
acagggttatcaaacctc

SEQ ID NO: 869 - DNA, 17 bp, Artificial Sequence
  Synthetic oligonucleotide
actgccagttgaatctg

SEQ ID NO: 870 - DNA, 16 bp, Artificial Sequence
  Synthetic oligonucleotide
cacgtctgccgaaagc

SEQ ID NO: 871 - DNA, 18 bp, Artificial Sequence
  Synthetic oligonucleotide
cagtcaagcctcttctcg

SEQ ID NO: 872 - DNA, 17 bp, Artificial Sequence
  Synthetic oligonucleotide
aagaggctggatttttg

SEQ ID NO: 873 - DNA, 20 bp, Artificial Sequence
  Synthetic oligonucleotide
aacccttggttgaatagtct

SEQ ID NO: 874 - DNA, 20 bp, Artificial Sequence
  Synthetic oligonucleotide
aaaacgtactgccactaaag

SEQ ID NO: 875 - DNA, 24 bp, Artificial Sequence
  Synthetic oligonucleotide
atcaatattcttaaacacaaattc

SEQ ID NO: 876 - DNA, 17 bp, Artificial Sequence
  Synthetic oligonucleotide
acgattgcagcattgtt

SEQ ID NO: 877 - DNA, 19 bp, Artificial Sequence
  Synthetic oligonucleotide
atgaatctggcttgaaaac

SEQ ID NO: 878 - DNA, 18 bp, Artificial Sequence
  Synthetic oligonucleotide
cttggactgagatctttc

SEQ ID NO: 879 - DNA, 20 bp, Artificial Sequence
  Synthetic oligonucleotide
ccatcaagctttttcttttc

SEQ ID NO: 880 - DNA, 18 bp, Artificial Sequence
  Synthetic oligonucleotide
atgtgcctttcaactctc

SEQ ID NO: 881 - DNA, 19 bp, Artificial Sequence
  Synthetic oligonucleotide
aaggaagaccttaaattcc

SEQ ID NO: 882 - DNA, 20 bp, Artificial Sequence
  Synthetic oligonucleotide
atggaaccattacagatgct

SEQ ID NO: 883 - DNA, 20 bp, Artificial Sequence
  Synthetic oligonucleotide
aacagagcctaaaaaggaca

SEQ ID NO: 884 - DNA, 19 bp, Artificial Sequence
  Synthetic oligonucleotide
ttggcaatgttgttccttg

SEQ ID NO: 885 - DNA, 19 bp, Artificial Sequence
  Synthetic oligonucleotide
caagacgcagtattattgg

SEQ ID NO: 886 - DNA, 20 bp, Artificial Sequence
  Synthetic oligonucleotide
ttgttttgatcgcgccccac

SEQ ID NO: 887 - DNA, 18 bp, Artificial Sequence
  Synthetic oligonucleotide
actgtgcacttgaccctc

SEQ ID NO: 888 - DNA, 19 bp, Artificial Sequence
  Synthetic oligonucleotide
atgacaaagatccaaattt

SEQ ID NO: 889 - DNA, 22 bp, Artificial Sequence
  Synthetic oligonucleotide
cccagtccctacttattgttaa

SEQ ID NO: 890 - DNA, 19 bp, Artificial Sequence
  Synthetic oligonucleotide
ccataaagccatcaagctt

SEQ ID NO: 891 - DNA, 18 bp, Artificial Sequence
  Synthetic oligonucleotide
ccaagggttgaaaagaaa

SEQ ID NO: 892 - DNA, 16 bp, Artificial Sequence
  Synthetic oligonucleotide
atctggcttgaaaacc

SEQ ID NO: 893 - DNA, 19 bp, Artificial Sequence
  Synthetic oligonucleotide
ccagaacaaacccaaggaa

SEQ ID NO: 894 - DNA, 15 bp, Artificial Sequence
  Synthetic oligonucleotide
cctcagggtttttcg

SEQ ID NO: 895 - DNA, 19 bp, Artificial Sequence
  Synthetic oligonucleotide
tcgtggtggtgacggtaaa

SEQ ID NO: 896 - DNA, 25 bp, Artificial Sequence
  Synthetic oligonucleotide
taggcgtgtgcttagaatatatttt

SEQ ID NO: 897 - DNA, 18 bp, Artificial Sequence
  Synthetic oligonucleotide
cagcaaactgtgactctt

SEQ ID NO: 898 - DNA, 18 bp, Artificial Sequence
  Synthetic oligonucleotide
ctcagtggaagcaaaata

SEQ ID NO: 899 - DNA, 18 bp, Artificial Sequence
  Synthetic oligonucleotide
caagctggttcaatctgt

SEQ ID NO: 900 - DNA, 16 bp, Artificial Sequence
  Synthetic oligonucleotide
cctgagggagatcacg

SEQ ID NO: 901 - DNA, 18 bp, Artificial Sequence
  Synthetic oligonucleotide
tagtgcgaccacccttac

SEQ ID NO: 902 - DNA, 23 bp, Artificial Sequence
  Synthetic oligonucleotide
taggcgtgtgcttagaatatatt

SEQ ID NO: 903 - DNA, 18 bp, Artificial Sequence
  Synthetic oligonucleotide
ctggagttgaatttcttg

SEQ ID NO: 904 - DNA, 20 bp, Artificial Sequence
  Synthetic oligonucleotide
atgccgtctttgttagcacc

SEQ ID NO: 905 - DNA, 17 bp, Artificial Sequence
  Synthetic oligonucleotide
actagtggcaataaaac

SEQ ID NO: 906 - DNA, 19 bp, Artificial Sequence
  Synthetic oligonucleotide
ttgggttccacgtgctagc

SEQ ID NO: 907 - DNA, 24 bp, Artificial Sequence
  Synthetic oligonucleotide
cccagtccctacttattgttaata

SEQ ID NO: 908 - DNA, 23 bp, Artificial Sequence
  Synthetic oligonucleotide
acgctactaatgttgttattaaa

SEQ ID NO: 909 - DNA, 16 bp, Artificial Sequence
  Synthetic oligonucleotide
ccctgacaaagttttc

SEQ ID NO: 910 - DNA, 19 bp, Artificial Sequence
  Synthetic oligonucleotide
atgctgttgttaaaattta

SEQ ID NO: 911 - DNA, 18 bp, Artificial Sequence
  Synthetic oligonucleotide
ataagaggctggattttt

SEQ ID NO: 912 - DNA, 19 bp, Artificial Sequence
  Synthetic oligonucleotide
cagaagaaacagcaaactg

SEQ ID NO: 913 - DNA, 19 bp, Artificial Sequence
  Synthetic oligonucleotide
ctactgctgcctggagttg

SEQ ID NO: 914 - DNA, 20 bp, Artificial Sequence
  Synthetic oligonucleotide
atggaatttaagggaaatac

SEQ ID NO: 915 - DNA, 18 bp, Artificial Sequence
  Synthetic oligonucleotide
acaaagatccaaatttca

SEQ ID NO: 916 - DNA, 18 bp, Artificial Sequence
  Synthetic oligonucleotide
ctgctgcttgacagattg

SEQ ID NO: 917 - DNA, 20 bp, Artificial Sequence
  Synthetic oligonucleotide
taggcgtgtgcttagaatat

SEQ ID NO: 918 - DNA, 14 bp, Artificial Sequence
  Synthetic oligonucleotide
cagcagcagatttc

SEQ ID NO: 919 - DNA, 21 bp, Artificial Sequence
  Synthetic oligonucleotide
ctgccactaaagcatacaatg

SEQ ID NO: 920 - DNA, 21 bp, Artificial Sequence
  Synthetic oligonucleotide
taggcgtgtgcttagaatata

SEQ ID NO: 921 - DNA, 16 bp, Artificial Sequence
  Synthetic oligonucleotide
catcaccgccattgcc

SEQ ID NO: 922 - DNA, 20 bp, Artificial Sequence
  Synthetic oligonucleotide
cacaaattccctaagatttt

SEQ ID NO: 923 - DNA, 19 bp, Artificial Sequence
  Synthetic oligonucleotide
ccaccaacagagcctaaaa

SEQ ID NO: 924 - DNA, 16 bp, Artificial Sequence
  Synthetic oligonucleotide
tcagtgccacaaaatt

SEQ ID NO: 925 - DNA, 23 bp, Artificial Sequence
  Synthetic oligonucleotide
tactactttagattcgaagaccc

SEQ ID NO: 926 - DNA, 21 bp, Artificial Sequence
  Synthetic oligonucleotide
cagccttttcttatggacctt

SEQ ID NO: 927 - DNA, 21 bp, Artificial Sequence
  Synthetic oligonucleotide
cttgaatacaccaaaagatca

SEQ ID NO: 928 - DNA, 21 bp, Artificial Sequence
  Synthetic oligonucleotide
aagtctgtgaatttcaatttt

SEQ ID NO: 929 - DNA, 20 bp, Artificial Sequence
  Synthetic oligonucleotide
acccataaagccatcaagct

SEQ ID NO: 930 - DNA, 17 bp, Artificial Sequence
  Synthetic oligonucleotide
ctagtgcgaataattgc

SEQ ID NO: 931 - DNA, 19 bp, Artificial Sequence
  Synthetic oligonucleotide
caaaacaacgtcggcccca

SEQ ID NO: 932 - DNA, 16 bp, Artificial Sequence
  Synthetic oligonucleotide
aagaggctggattttt

SEQ ID NO: 933 - DNA, 23 bp, Artificial Sequence
  Synthetic oligonucleotide
ctagtggcaataaaacaagaaaa

SEQ ID NO: 934 - DNA, 19 bp, Artificial Sequence
  Synthetic oligonucleotide
acaaccagaactcaattac

SEQ ID NO: 935 - DNA, 19 bp, Artificial Sequence
  Synthetic oligonucleotide
atggaaccaagtaacattg

SEQ ID NO: 936 - DNA, 19 bp, Artificial Sequence
  Synthetic oligonucleotide
acgtactgccactaaagca

SEQ ID NO: 937 - DNA, 20 bp, Artificial Sequence
  Synthetic oligonucleotide
ctgtaatggttccattttca

SEQ ID NO: 938 - DNA, 19 bp, Artificial Sequence
  Synthetic oligonucleotide
tagcatggaaccaagtaac

SEQ ID NO: 939 - DNA, 18 bp, Artificial Sequence
  Synthetic oligonucleotide
tctgagggtccaccaaac

SEQ ID NO: 940 - DNA, 18 bp, Artificial Sequence
  Synthetic oligonucleotide
caatcgtgctacaacttc

SEQ ID NO: 941 - DNA, 22 bp, Artificial Sequence
  Synthetic oligonucleotide
ttggaaaagaaaggtaagaaca

SEQ ID NO: 942 - DNA, 14 bp, Artificial Sequence
  Synthetic oligonucleotide
tccccaaaatttcc

SEQ ID NO: 943 - DNA, 20 bp, Artificial Sequence
  Synthetic oligonucleotide
aaggctgatgaaactcaagc

SEQ ID NO: 944 - DNA, 18 bp, Artificial Sequence
  Synthetic oligonucleotide
ttgaaggtgtcaaatttc

SEQ ID NO: 945 - DNA, 18 bp, Artificial Sequence
  Synthetic oligonucleotide
tcgtctggtagctcttcg

SEQ ID NO: 946 - DNA, 18 bp, Artificial Sequence
  Synthetic oligonucleotide
ctcctgctagaatggctg

SEQ ID NO: 947 - DNA, 18 bp, Artificial Sequence
  Synthetic oligonucleotide
ctgcgtagaagccttttg

SEQ ID NO: 948 - DNA, 17 bp, Artificial Sequence
  Synthetic oligonucleotide
atggcaaggaagacctt

SEQ ID NO: 949 - DNA, 19 bp, Artificial Sequence
  Synthetic oligonucleotide
cccatatgatgccgtcttt

SEQ ID NO: 950 - DNA, 19 bp, Artificial Sequence
  Synthetic oligonucleotide
cagtccaagatggtatttc

SEQ ID NO: 951 - DNA, 18 bp, Artificial Sequence
  Synthetic oligonucleotide
cctgcatacactaattct

SEQ ID NO: 952 - DNA, 20 bp, Artificial Sequence
  Synthetic oligonucleotide
ctgcatacactaattctttc

SEQ ID NO: 953 - DNA, 19 bp, Artificial Sequence
  Synthetic oligonucleotide
ttgtttggagaaatcatcc

SEQ ID NO: 954 - DNA, 16 bp, Artificial Sequence
  Synthetic oligonucleotide
catgctggacaataaa

SEQ ID NO: 955 - DNA, 23 bp, Artificial Sequence
  Synthetic oligonucleotide
tcaatattcttaaacacaaattc

SEQ ID NO: 956 - DNA, 19 bp, Artificial Sequence
  Synthetic oligonucleotide
ttgcttccactgagaagtc

SEQ ID NO: 957 - DNA, 21 bp, Artificial Sequence
  Synthetic oligonucleotide
ctgtcctaccatttaatgatg

SEQ ID NO: 958 - DNA, 17 bp, Artificial Sequence
  Synthetic oligonucleotide
ttcccaccaacagagcc

SEQ ID NO: 959 - DNA, 20 bp, Artificial Sequence
  Synthetic oligonucleotide
aatgatggtgtttattttgc

SEQ ID NO: 960 - DNA, 20 bp, Artificial Sequence
  Synthetic oligonucleotide
attcttaaacacaaattccc

SEQ ID NO: 961 - DNA, 18 bp, Artificial Sequence
  Synthetic oligonucleotide
acctgaagaagaatcacc

SEQ ID NO: 962 - DNA, 26 bp, Artificial Sequence
  Synthetic oligonucleotide
accatcaatattcttaaacacaaatt

SEQ ID NO: 963 - DNA, 19 bp, Artificial Sequence
  Synthetic oligonucleotide
aagtagtggcaccttcttt

SEQ ID NO: 964 - DNA, 20 bp, Artificial Sequence
  Synthetic oligonucleotide
cctgagttgaatgtaaaact

SEQ ID NO: 965 - DNA, 24 bp, Artificial Sequence
  Synthetic oligonucleotide
cagtccctacttattgttaataac

SEQ ID NO: 966 - DNA, 15 bp, Artificial Sequence
  Synthetic oligonucleotide
ctggtccccaaaatt

SEQ ID NO: 967 - DNA, 17 bp, Artificial Sequence
  Synthetic oligonucleotide
ccacgtgtgaaagaatt

SEQ ID NO: 968 - DNA, 18 bp, Artificial Sequence
  Synthetic oligonucleotide
cagcgttcttcggaatgt

SEQ ID NO: 969 - DNA, 23 bp, Artificial Sequence
  Synthetic oligonucleotide
cccagtccctacttattgttaat

SEQ ID NO: 970 - DNA, 19 bp, Artificial Sequence
  Synthetic oligonucleotide
acagatgctgtagactgtg

SEQ ID NO: 971 - DNA, 20 bp, Artificial Sequence
  Synthetic oligonucleotide
cttgccgaataccataatga

SEQ ID NO: 972 - DNA, 19 bp, Artificial Sequence
  Synthetic oligonucleotide
cttggactgagatctttca

SEQ ID NO: 973 - DNA, 18 bp, Artificial Sequence
  Synthetic oligonucleotide
caattgtttggagaaatc

SEQ ID NO: 974 - DNA, 16 bp, Artificial Sequence
  Synthetic oligonucleotide
cccaccaacagagcct

SEQ ID NO: 975 - DNA, 22 bp, Artificial Sequence
  Synthetic oligonucleotide
cagttcctaggtagtagaaata

SEQ ID NO: 976 - DNA, 19 bp, Artificial Sequence
  Synthetic oligonucleotide
ttaccctgacaaagttttc

SEQ ID NO: 977 - DNA, 17 bp, Artificial Sequence
  Synthetic oligonucleotide
aatcgtgctacaacttc

SEQ ID NO: 978 - DNA, 14 bp, Artificial Sequence
  Synthetic oligonucleotide
cacccgcaatcctg

SEQ ID NO: 979 - DNA, 20 bp, Artificial Sequence
  Synthetic oligonucleotide
cacaatttgcccccagcgct

SEQ ID NO: 980 - DNA, 22 bp, Artificial Sequence
  Synthetic oligonucleotide
ccactaaagcatacaatgtaac

SEQ ID NO: 981 - DNA, 16 bp, Artificial Sequence
  Synthetic oligonucleotide
tttgccgaggcttctt

SEQ ID NO: 982 - DNA, 20 bp, Artificial Sequence
  Synthetic oligonucleotide
actaagaggtttgataaccc

SEQ ID NO: 983 - DNA, 17 bp, Artificial Sequence
  Synthetic oligonucleotide
cttgactgccgcctctg

SEQ ID NO: 984 - DNA, 21 bp, Artificial Sequence
  Synthetic oligonucleotide
tacaatgtaacacaagctttc

SEQ ID NO: 985 - DNA, 19 bp, Artificial Sequence
  Synthetic oligonucleotide
atgctggacaataaatttt

SEQ ID NO: 986 - DNA, 20 bp, Artificial Sequence
  Synthetic oligonucleotide
cccagagacatgtatagcat

SEQ ID NO: 987 - DNA, 17 bp, Artificial Sequence
  Synthetic oligonucleotide
ctgcggtaaggcttgag

SEQ ID NO: 988 - DNA, 20 bp, Artificial Sequence
  Synthetic oligonucleotide
atgtctctcagccttttctt

SEQ ID NO: 989 - DNA, 18 bp, Artificial Sequence
  Synthetic oligonucleotide
aagtgtacgttgaaatcc

SEQ ID NO: 990 - DNA, 19 bp, Artificial Sequence
  Synthetic oligonucleotide
tacttaccccaaaatgctg

SEQ ID NO: 991 - DNA, 17 bp, Artificial Sequence
  Synthetic oligonucleotide
ttgccgaggcttcttag

SEQ ID NO: 992 - DNA, 20 bp, Artificial Sequence
  Synthetic oligonucleotide
cagtccaagatggtatttct

SEQ ID NO: 993 - DNA, 20 bp, Artificial Sequence
  Synthetic oligonucleotide
atgcgtcaatatgcttattc

SEQ ID NO: 994 - DNA, 22 bp, Artificial Sequence
  Synthetic oligonucleotide
aagcaagtaaagtttgaaacct

SEQ ID NO: 995 - DNA, 22 bp, Artificial Sequence
  Synthetic oligonucleotide
ttcttagaagcctcagcagcag

SEQ ID NO: 996 - DNA, 16 bp, Artificial Sequence
  Synthetic oligonucleotide
ctccctcagggttttt

SEQ ID NO: 997 - DNA, 18 bp, Artificial Sequence
  Synthetic oligonucleotide
cctcaaggaacaacattg

SEQ ID NO: 998 - DNA, 15 bp, Artificial Sequence
  Synthetic oligonucleotide
cagacgggcgatttt

SEQ ID NO: 999 - DNA, 17 bp, Artificial Sequence
  Synthetic oligonucleotide
tctcagtgccacaaaat

SEQ ID NO: 1000 - DNA, 16 bp, Artificial Sequence
  Synthetic oligonucleotide
tctggcttgaaaacca

SEQ ID NO: 1001 - DNA, 20 bp, Artificial Sequence
  Synthetic oligonucleotide
cccttggttgaatagtcttg

SEQ ID NO: 1002 - DNA, 16 bp, Artificial Sequence
  Synthetic oligonucleotide
tagcaggagaagttcc

SEQ ID NO: 1003 - DNA, 16 bp, Artificial Sequence
  Synthetic oligonucleotide
ccagctgtccaacctg

SEQ ID NO: 1004 - DNA, 18 bp, Artificial Sequence
  Synthetic oligonucleotide
acattggcacccgcaatc

SEQ ID NO: 1005 - DNA, 17 bp, Artificial Sequence
  Synthetic oligonucleotide
ttgtggcactgagaatt

SEQ ID NO: 1006 - DNA, 23 bp, Artificial Sequence
  Synthetic oligonucleotide
tctatgtaaagcaagtaaagttt

SEQ ID NO: 1007 - DNA, 24 bp, Artificial Sequence
  Synthetic oligonucleotide
taggcgtgtgcttagaatatattt

SEQ ID NO: 1008 - DNA, 17 bp, Artificial Sequence
  Synthetic oligonucleotide
cagcatgtcacaattca

SEQ ID NO: 1009 - DNA, 17 bp, Artificial Sequence
  Synthetic oligonucleotide
cattgtggtgaaacttc

SEQ ID NO: 1010 - DNA, 22 bp, Artificial Sequence
  Synthetic oligonucleotide
ctagtggcaataaaacaagaaa

SEQ ID NO: 1011 - DNA, 22 bp, Artificial Sequence
  Synthetic oligonucleotide
tcttcaacctaggacttttcta

SEQ ID NO: 1012 - DNA, 15 bp, Artificial Sequence
  Synthetic oligonucleotide
ttgcggccaatgttt

SEQ ID NO: 1013 - DNA, 19 bp, Artificial Sequence
  Synthetic oligonucleotide
atgctcaggtcctacttct

SEQ ID NO: 1014 - DNA, 21 bp, Artificial Sequence
  Synthetic oligonucleotide
ttttctacagtgaaggatttc

SEQ ID NO: 1015 - DNA, 17 bp, Artificial Sequence
  Synthetic oligonucleotide
ccagaactcaattaccc

SEQ ID NO: 1016 - DNA, 19 bp, Artificial Sequence
  Synthetic oligonucleotide
caaagtgtacgttgaaatc

SEQ ID NO: 1017 - DNA, 19 bp, Artificial Sequence
  Synthetic oligonucleotide
ttggcctttaccagacatt

SEQ ID NO: 1018 - DNA, 17 bp, Artificial Sequence
  Synthetic oligonucleotide
ccaggagtcaaataact

SEQ ID NO: 1019 - DNA, 23 bp, Artificial Sequence
  Synthetic oligonucleotide
taaagtctgtgaatttcaatttt

SEQ ID NO: 1020 - DNA, 17 bp, Artificial Sequence
  Synthetic oligonucleotide
cacaggtgccatcaaat

SEQ ID NO: 1021 - DNA, 19 bp, Artificial Sequence
  Synthetic oligonucleotide
catggaaccaagtaacatt

SEQ ID NO: 1022 - DNA, 17 bp, Artificial Sequence
  Synthetic oligonucleotide
ccgtctgccatgaagtt

SEQ ID NO: 1023 - DNA, 18 bp, Artificial Sequence
  Synthetic oligonucleotide
ctgtcactaagaaatctg

SEQ ID NO: 1024 - DNA, 18 bp, Artificial Sequence
  Synthetic oligonucleotide
aacaacaaggccaaactg

SEQ ID NO: 1025 - DNA, 16 bp, Artificial Sequence
  Synthetic oligonucleotide
atgcgcgacattccga

SEQ ID NO: 1026 - DNA, 20 bp, Artificial Sequence
  Synthetic oligonucleotide
ctgagttgaatgtaaaactg

SEQ ID NO: 1027 - DNA, 20 bp, Artificial Sequence
  Synthetic oligonucleotide
cttgaatacaccaaaagatc

SEQ ID NO: 1028 - DNA, 17 bp, Artificial Sequence
  Synthetic oligonucleotide
ttgcgtcaccaaatgaa

SEQ ID NO: 1029 - DNA, 21 bp, Artificial Sequence
  Synthetic oligonucleotide
aagataacccacataataagc

SEQ ID NO: 1030 - DNA, 17 bp, Artificial Sequence
  Synthetic oligonucleotide
caaggcgttccaattaa

SEQ ID NO: 1031 - DNA, 19 bp, Artificial Sequence
  Synthetic oligonucleotide
aaagtagtaccaaaaatcc

SEQ ID NO: 1032 - DNA, 19 bp, Artificial Sequence
  Synthetic oligonucleotide
cctgcatacactaattctt

SEQ ID NO: 1033 - DNA, 24 bp, Artificial Sequence
  Synthetic oligonucleotide
ccagtccctacttattgttaataa

SEQ ID NO: 1034 - DNA, 15 bp, Artificial Sequence
  Synthetic oligonucleotide
caccaccacgaattc

SEQ ID NO: 1035 - DNA, 17 bp, Artificial Sequence
  Synthetic oligonucleotide
acaacaaggccaaactg

SEQ ID NO: 1036 - DNA, 18 bp, Artificial Sequence
  Synthetic oligonucleotide
ttgcgggtgccaatgtga

SEQ ID NO: 1037 - DNA, 16 bp, Artificial Sequence
  Synthetic oligonucleotide
ccaggagtcaaataac

SEQ ID NO: 1038 - DNA, 24 bp, Artificial Sequence
  Synthetic oligonucleotide
aacgctactaatgttgttattaaa

SEQ ID NO: 1039 - DNA, 19 bp, Artificial Sequence
  Synthetic oligonucleotide
acaaaatttggacattccc

SEQ ID NO: 1040 - DNA, 19 bp, Artificial Sequence
  Synthetic oligonucleotide
ttggctactaccgaagagc

SEQ ID NO: 1041 - DNA, 20 bp, Artificial Sequence
  Synthetic oligonucleotide
ctacagtgaaggatttcaac

SEQ ID NO: 1042 - DNA, 16 bp, Artificial Sequence
  Synthetic oligonucleotide
acagcctccaaaggca

SEQ ID NO: 1043 - DNA, 20 bp, Artificial Sequence
  Synthetic oligonucleotide
caaccaacataagagaacac

SEQ ID NO: 1044 - DNA, 19 bp, Artificial Sequence
  Synthetic oligonucleotide
tagtggcagtacgtttttg

SEQ ID NO: 1045 - DNA, 18 bp, Artificial Sequence
  Synthetic oligonucleotide
tactgccagttgaatctg

SEQ ID NO: 1046 - DNA, 18 bp, Artificial Sequence
  Synthetic oligonucleotide
atggattgttgcaattgt

SEQ ID NO: 1047 - DNA, 19 bp, Artificial Sequence
  Synthetic oligonucleotide
ctcgaggacaaggcgttcc

SEQ ID NO: 1048 - DNA, 20 bp, Artificial Sequence
  Synthetic oligonucleotide
catcagccttcttctttttg

SEQ ID NO: 1049 - DNA, 23 bp, Artificial Sequence
  Synthetic oligonucleotide
caacacgagcagcctctgatgca

SEQ ID NO: 1050 - DNA, 19 bp, Artificial Sequence
  Synthetic oligonucleotide
agcgctagcacgtggaacc

SEQ ID NO: 1051 - DNA, 20 bp, Artificial Sequence
  Synthetic oligonucleotide
cccaaaatgctgttgttaaa

SEQ ID NO: 1052 - DNA, 17 bp, Artificial Sequence
  Synthetic oligonucleotide
tagggactgggtcttcg

SEQ ID NO: 1053 - DNA, 15 bp, Artificial Sequence
  Synthetic oligonucleotide
cccagcgcttcagcg

SEQ ID NO: 1054 - DNA, 18 bp, Artificial Sequence
  Synthetic oligonucleotide
tccagttgcgtcaccaaa

SEQ ID NO: 1055 - DNA, 18 bp, Artificial Sequence
  Synthetic oligonucleotide
cagagacagaagaaacag

SEQ ID NO: 1056 - DNA, 22 bp, Artificial Sequence
  Synthetic oligonucleotide
cttgttcttacctttcttttcc

SEQ ID NO: 1057 - DNA, 22 bp, Artificial Sequence
  Synthetic oligonucleotide
tttgccaataggtattaacatc

SEQ ID NO: 1058 - DNA, 17 bp, Artificial Sequence
  Synthetic oligonucleotide
actgctgcctggagttg

SEQ ID NO: 1059 - DNA, 17 bp, Artificial Sequence
  Synthetic oligonucleotide
cagcatcaccgccattg

SEQ ID NO: 1060 - DNA, 20 bp, Artificial Sequence
  Synthetic oligonucleotide
aattcgtctggtagctcttc

SEQ ID NO: 1061 - DNA, 18 bp, Artificial Sequence
  Synthetic oligonucleotide
tagggaagtccagcttct

SEQ ID NO: 1062 - DNA, 20 bp, Artificial Sequence
  Synthetic oligonucleotide
tcgaattctacccataaagc

SEQ ID NO: 1063 - DNA, 21 bp, Artificial Sequence
  Synthetic oligonucleotide
ttacaaccagaactcaattac

SEQ ID NO: 1064 - DNA, 20 bp, Artificial Sequence
  Synthetic oligonucleotide
caaattccctaagatttttg

SEQ ID NO: 1065 - DNA, 18 bp, Artificial Sequence
  Synthetic oligonucleotide
cccaccaacagagcctaa

SEQ ID NO: 1066 - DNA, 19 bp, Artificial Sequence
  Synthetic oligonucleotide
accattggtcccagagaca

SEQ ID NO: 1067 - DNA, 18 bp, Artificial Sequence
  Synthetic oligonucleotide
acagagcctaaaaaggac

SEQ ID NO: 1068 - DNA, 18 bp, Artificial Sequence
  Synthetic oligonucleotide
actgaggatctgaaaact

SEQ ID NO: 1069 - DNA, 20 bp, Artificial Sequence
  Synthetic oligonucleotide
aaggaaaacagggtaatttc

SEQ ID NO: 1070 - DNA, 21 bp, Artificial Sequence
  Synthetic oligonucleotide
ctactttagattcgaagaccc

SEQ ID NO: 1071 - DNA, 19 bp, Artificial Sequence
  Synthetic oligonucleotide
ctagtggcaataaaacaag

SEQ ID NO: 1072 - DNA, 18 bp, Artificial Sequence
  Synthetic oligonucleotide
ttgtggcactgagaattt

SEQ ID NO: 1073 - DNA, 17 bp, Artificial Sequence
  Synthetic oligonucleotide
ccgcaaattgcacaatt

SEQ ID NO: 1074 - DNA, 15 bp, Artificial Sequence
  Synthetic oligonucleotide
acccaaggaaatttt

SEQ ID NO: 1075 - DNA, 19 bp, Artificial Sequence
  Synthetic oligonucleotide
catgagagttgaaaggcac

SEQ ID NO: 1076 - DNA, 16 bp, Artificial Sequence
  Synthetic oligonucleotide
ttgcccccagcgcttc

SEQ ID NO: 1077 - DNA, 19 bp, Artificial Sequence
  Synthetic oligonucleotide
atccagttgcgtcaccaaa

SEQ ID NO: 1078 - DNA, 18 bp, Artificial Sequence
  Synthetic oligonucleotide
aaggctgagagacatatt

SEQ ID NO: 1079 - DNA, 18 bp, Artificial Sequence
  Synthetic oligonucleotide
caaggctccctcagttgc

SEQ ID NO: 1080 - DNA, 21 bp, Artificial Sequence
  Synthetic oligonucleotide
tctctagtcagtgtgttaatc

SEQ ID NO: 1081 - DNA, 19 bp, Artificial Sequence
  Synthetic oligonucleotide
accctgtcctaccatttaa

SEQ ID NO: 1082 - DNA, 18 bp, Artificial Sequence
  Synthetic oligonucleotide
ttggtcccagagacatgt

SEQ ID NO: 1083 - DNA, 18 bp, Artificial Sequence
  Synthetic oligonucleotide
cagcttgagagcaaaatg

SEQ ID NO: 1084 - DNA, 19 bp, Artificial Sequence
  Synthetic oligonucleotide
aaggctgatgaaactcaag

SEQ ID NO: 1085 - DNA, 15 bp, Artificial Sequence
  Synthetic oligonucleotide
ctctgcggtaaggct

SEQ ID NO: 1086 - DNA, 17 bp, Artificial Sequence
  Synthetic oligonucleotide
cttggttcaccgctctc

SEQ ID NO: 1087 - DNA, 19 bp, Artificial Sequence
  Synthetic oligonucleotide
tcgaggacaaggcgttcca

SEQ ID NO: 1088 - DNA, 15 bp, Artificial Sequence
  Synthetic oligonucleotide
tagcacgtggaaccc

SEQ ID NO: 1089 - DNA, 18 bp, Artificial Sequence
  Synthetic oligonucleotide
ttggaggctgtgtgttct

SEQ ID NO: 1090 - DNA, 20 bp, Artificial Sequence
  Synthetic oligonucleotide
cctcatcacgtagtcgcaac

SEQ ID NO: 1091 - DNA, 18 bp, Artificial Sequence
  Synthetic oligonucleotide
aagaggctggatttttgg

SEQ ID NO: 1092 - DNA, 21 bp, Artificial Sequence
  Synthetic oligonucleotide
ttaatacctattggcaaatct

SEQ ID NO: 1093 - DNA, 19 bp, Artificial Sequence
  Synthetic oligonucleotide
aagccttaccgcagagaca

SEQ ID NO: 1094 - DNA, 18 bp, Artificial Sequence
  Synthetic oligonucleotide
acgtttttgccgaggctt

SEQ ID NO: 1095 - DNA, 23 bp, Artificial Sequence
  Synthetic oligonucleotide
tagtggcaataaaacaagaaaaa

SEQ ID NO: 1096 - DNA, 18 bp, Artificial Sequence
  Synthetic oligonucleotide
tcttggactgagatcttt

SEQ ID NO: 1097 - DNA, 20 bp, Artificial Sequence
  Synthetic oligonucleotide
tccaacttttgttgtttttg

SEQ ID NO: 1098 - DNA, 18 bp, Artificial Sequence
  Synthetic oligonucleotide
ttgtatgcgtcaatatgc

SEQ ID NO: 1099 - DNA, 19 bp, Artificial Sequence
  Synthetic oligonucleotide
atgtctctcagccttttct

SEQ ID NO: 1100 - DNA, 20 bp, Artificial Sequence
  Synthetic oligonucleotide
ctgcagatttggatgatttc

SEQ ID NO: 1101 - DNA, 16 bp, Artificial Sequence
  Synthetic oligonucleotide
caatgctgcaatcgtg

SEQ ID NO: 1102 - DNA, 19 bp, Artificial Sequence
  Synthetic oligonucleotide
ttgaaggtgtcaaatttct

SEQ ID NO: 1103 - DNA, 18 bp, Artificial Sequence
  Synthetic oligonucleotide
atcattgtggtgaaactt

SEQ ID NO: 1104 - DNA, 17 bp, Artificial Sequence
  Synthetic oligonucleotide
accctgacaaagttttc

SEQ ID NO: 1105 - DNA, 16 bp, Artificial Sequence
  Synthetic oligonucleotide
cctggtccccaaaatt

SEQ ID NO: 1106 - DNA, 16 bp, Artificial Sequence
  Synthetic oligonucleotide
tcgcgccccactgcgt

SEQ ID NO: 1107 - DNA, 20 bp, Artificial Sequence
  Synthetic oligonucleotide
ataggcgtgtgcttagaata

SEQ ID NO: 1108 - DNA, 18 bp, Artificial Sequence
  Synthetic oligonucleotide
ctgagggtccaccaaacg

SEQ ID NO: 1109 - DNA, 20 bp, Artificial Sequence
  Synthetic oligonucleotide
ctacttgtggttacttaccc

SEQ ID NO: 1110 - DNA, 21 bp, Artificial Sequence
  Synthetic oligonucleotide
cctgtcctaccatttaatgat

SEQ ID NO: 1111 - DNA, 19 bp, Artificial Sequence
  Synthetic oligonucleotide
atcacgtagtcgcaacagt

SEQ ID NO: 1112 - DNA, 18 bp, Artificial Sequence
  Synthetic oligonucleotide
aagtccagcttctggccc

SEQ ID NO: 1113 - DNA, 24 bp, Artificial Sequence
  Synthetic oligonucleotide
acaaacattgttcgtttagttgtt

SEQ ID NO: 1114 - DNA, 21 bp, Artificial Sequence
  Synthetic oligonucleotide
aaatggatcattacaaaattg

SEQ ID NO: 1115 - DNA, 17 bp, Artificial Sequence
  Synthetic oligonucleotide
ttgccagccattctagc

SEQ ID NO: 1116 - DNA, 18 bp, Artificial Sequence
  Synthetic oligonucleotide
ccgaagaacgctgaagcg

SEQ ID NO: 1117 - DNA, 19 bp, Artificial Sequence
  Synthetic oligonucleotide
caatagtgcgaccaccctt

SEQ ID NO: 1118 - DNA, 20 bp, Artificial Sequence
  Synthetic oligonucleotide
ctgattagttcctggtcccc

SEQ ID NO: 1119 - DNA, 19 bp, Artificial Sequence
  Synthetic oligonucleotide
atgtgcctttcaactctca

SEQ ID NO: 1120 - DNA, 24 bp, Artificial Sequence
  Synthetic oligonucleotide
ccttcttctttttgtcctttttag

SEQ ID NO: 1121 - DNA, 16 bp, Artificial Sequence
  Synthetic oligonucleotide
ttgccagccattctag

SEQ ID NO: 1122 - DNA, 19 bp, Artificial Sequence
  Synthetic oligonucleotide
tcagcgttcttcggaatgt

SEQ ID NO: 1123 - DNA, 21 bp, Artificial Sequence
  Synthetic oligonucleotide
cccagttcctaggtagtagaa

SEQ ID NO: 1124 - DNA, 24 bp, Artificial Sequence
  Synthetic oligonucleotide
aagataacccacataataagctgc

SEQ ID NO: 1125 - DNA, 17 bp, Artificial Sequence
  Synthetic oligonucleotide
cacgtgtgaaagaatta

SEQ ID NO: 1126 - DNA, 22 bp, Artificial Sequence
  Synthetic oligonucleotide
tatgtaaagcaagtaaagtttg

SEQ ID NO: 1127 - DNA, 16 bp, Artificial Sequence
  Synthetic oligonucleotide
cccactgcgttctcca

SEQ ID NO: 1128 - DNA, 19 bp, Artificial Sequence
  Synthetic oligonucleotide
tctgtgaatttcaattttg

SEQ ID NO: 1129 - DNA, 20 bp, Artificial Sequence
  Synthetic oligonucleotide
aagtgtacgttgaaatcctt

SEQ ID NO: 1130 - DNA, 24 bp, Artificial Sequence
  Synthetic oligonucleotide
tcaacctaggacttttctattaaa

SEQ ID NO: 1131 - DNA, 19 bp, Artificial Sequence
  Synthetic oligonucleotide
tacacaggtgccatcaaat

SEQ ID NO: 1132 - DNA, 18 bp, Artificial Sequence
  Synthetic oligonucleotide
acctgagcatagtcttgc

SEQ ID NO: 1133 - DNA, 19 bp, Artificial Sequence
  Synthetic oligonucleotide
acattccccattgaaggtg

SEQ ID NO: 1134 - DNA, 18 bp, Artificial Sequence
  Synthetic oligonucleotide
tcaagctggttcaatctg

SEQ ID NO: 1135 - DNA, 18 bp, Artificial Sequence
  Synthetic oligonucleotide
ctgaggatctgaaaactt

SEQ ID NO: 1136 - DNA, 17 bp, Artificial Sequence
  Synthetic oligonucleotide
ctgcgtcttggttcacc

SEQ ID NO: 1137 - DNA, 21 bp, Artificial Sequence
  Synthetic oligonucleotide
ttgcacaatttgcccccagcg

SEQ ID NO: 1138 - DNA, 17 bp, Artificial Sequence
  Synthetic oligonucleotide
tttttgccgaggcttct

SEQ ID NO: 1139 - DNA, 18 bp, Artificial Sequence
  Synthetic oligonucleotide
accctctctcagaaacaa

SEQ ID NO: 1140 - DNA, 19 bp, Artificial Sequence
  Synthetic oligonucleotide
ttgccgaggcttcttagaa

SEQ ID NO: 1141 - DNA, 21 bp, Artificial Sequence
  Synthetic oligonucleotide
atccaatttgatggcacctgt

SEQ ID NO: 1142 - DNA, 19 bp, Artificial Sequence
  Synthetic oligonucleotide
aacaaacccaaggaaattt

SEQ ID NO: 1143 - DNA, 19 bp, Artificial Sequence
  Synthetic oligonucleotide
aatacctattggcaaatct

SEQ ID NO: 1144 - DNA, 19 bp, Artificial Sequence
  Synthetic oligonucleotide
ccacaatgatcacacttca

SEQ ID NO: 1145 - DNA, 20 bp, Artificial Sequence
  Synthetic oligonucleotide
cccaataggcacacttgtta

SEQ ID NO: 1146 - DNA, 18 bp, Artificial Sequence
  Synthetic oligonucleotide
taccctgacaaagttttc

SEQ ID NO: 1147 - DNA, 19 bp, Artificial Sequence
  Synthetic oligonucleotide
ttgtggcactgagaatttg

SEQ ID NO: 1148 - DNA, 15 bp, Artificial Sequence
  Synthetic oligonucleotide
ccatgccaatgcgcg

SEQ ID NO: 1149 - DNA, 20 bp, Artificial Sequence
  Synthetic oligonucleotide
attcaaaagtgcaattattc

SEQ ID NO: 1150 - DNA, 19 bp, Artificial Sequence
  Synthetic oligonucleotide
ctgttttccttcaaggtcc

SEQ ID NO: 1151 - DNA, 17 bp, Artificial Sequence
  Synthetic oligonucleotide
ctgctgcttgacagatt

SEQ ID NO: 1152 - DNA, 17 bp, Artificial Sequence
  Synthetic oligonucleotide
acgtggtccagaacaaa

SEQ ID NO: 1153 - DNA, 20 bp, Artificial Sequence
  Synthetic oligonucleotide
cttcaacctaggacttttct

SEQ ID NO: 1154 - DNA, 19 bp, Artificial Sequence
  Synthetic oligonucleotide
atgaatgcaaccaaatgtg

SEQ ID NO: 1155 - DNA, 15 bp, Artificial Sequence
  Synthetic oligonucleotide
cagtgccacaaaatt

SEQ ID NO: 1156 - DNA, 19 bp, Artificial Sequence
  Synthetic oligonucleotide
cacttgttatggcaaccaa

SEQ ID NO: 1157 - DNA, 18 bp, Artificial Sequence
  Synthetic oligonucleotide
acattgccaaaaggcttc

SEQ ID NO: 1158 - DNA, 19 bp, Artificial Sequence
  Synthetic oligonucleotide
aaggctgagagacatattc

SEQ ID NO: 1159 - DNA, 18 bp, Artificial Sequence
  Synthetic oligonucleotide
ccctgtcctaccatttaa

SEQ ID NO: 1160 - DNA, 17 bp, Artificial Sequence
  Synthetic oligonucleotide
ctagtgcgaataattgc

SEQ ID NO: 1161 - DNA, 19 bp, Artificial Sequence
  Synthetic oligonucleotide
taagcacacgcctattaat

SEQ ID NO: 1162 - DNA, 19 bp, Artificial Sequence
  Synthetic oligonucleotide
acccataaagccatcaagc

SEQ ID NO: 1163 - DNA, 18 bp, Artificial Sequence
  Synthetic oligonucleotide
caagtagtggcaccttct

SEQ ID NO: 1164 - DNA, 19 bp, Artificial Sequence
  Synthetic oligonucleotide
aaactgtcactaagaaatc

SEQ ID NO: 1165 - DNA, 19 bp, Artificial Sequence
  Synthetic oligonucleotide
ctgcgttctccattctggt

SEQ ID NO: 1166 - DNA, 18 bp, Artificial Sequence
  Synthetic oligonucleotide
atgctgttgttaaaattt

SEQ ID NO: 1167 - DNA, 16 bp, Artificial Sequence
  Synthetic oligonucleotide
attggcacccgcaatc

SEQ ID NO: 1168 - DNA, 17 bp, Artificial Sequence
  Synthetic oligonucleotide
cccgcaatcctgctaac

SEQ ID NO: 1169 - DNA, 16 bp, Artificial Sequence
  Synthetic oligonucleotide
cagcagcagatttctt

SEQ ID NO: 1170 - DNA, 20 bp, Artificial Sequence
  Synthetic oligonucleotide
tttgtatgcgtcaatatgct

SEQ ID NO: 1171 - DNA, 23 bp, Artificial Sequence
  Synthetic oligonucleotide
ctgcagcttattatgtgggttat

SEQ ID NO: 1172 - DNA, 19 bp, Artificial Sequence
  Synthetic oligonucleotide
aactctgaactcactttcc

SEQ ID NO: 1173 - DNA, 17 bp, Artificial Sequence
  Synthetic oligonucleotide
ccagttgcgtcaccaaa

SEQ ID NO: 1174 - DNA, 17 bp, Artificial Sequence
  Synthetic oligonucleotide
aagccacttgcgaattt

SEQ ID NO: 1175 - DNA, 17 bp, Artificial Sequence
  Synthetic oligonucleotide
aacaaggccaaactgtc

SEQ ID NO: 1176 - DNA, 18 bp, Artificial Sequence
  Synthetic oligonucleotide
atcctgctaacaatgctg

SEQ ID NO: 1177 - DNA, 20 bp, Artificial Sequence
  Synthetic oligonucleotide
aaactgaggatctgaaaact

SEQ ID NO: 1178 - DNA, 19 bp, Artificial Sequence
  Synthetic oligonucleotide
ctgttggtgggaatgtttt

SEQ ID NO: 1179 - DNA, 21 bp, Artificial Sequence
  Synthetic oligonucleotide
cagcttattatgtgggttatc

SEQ ID NO: 1180 - DNA, 20 bp, Artificial Sequence
  Synthetic oligonucleotide
ccaagtaacattggaaaaga

SEQ ID NO: 1181 - DNA, 19 bp, Artificial Sequence
  Synthetic oligonucleotide
ccagatgaccaaattggct

SEQ ID NO: 1182 - DNA, 18 bp, Artificial Sequence
  Synthetic oligonucleotide
ccttaccgcagagacaga

SEQ ID NO: 1183 - DNA, 17 bp, Artificial Sequence
  Synthetic oligonucleotide
accagctgtccaacctg

SEQ ID NO: 1184 - DNA, 20 bp, Artificial Sequence
  Synthetic oligonucleotide
caaggtttacccaataatac

SEQ ID NO: 1185 - DNA, 19 bp, Artificial Sequence
  Synthetic oligonucleotide
aaggcgttccaattaacac

SEQ ID NO: 1186 - DNA, 18 bp, Artificial Sequence
  Synthetic oligonucleotide
cctttaccagacattttg

SEQ ID NO: 1187 - DNA, 21 bp, Artificial Sequence
  Synthetic oligonucleotide
tcagccttcttctttttgtcc

SEQ ID NO: 1188 - DNA, 19 bp, Artificial Sequence
  Synthetic oligonucleotide
cctactgctgcctggagtt

SEQ ID NO: 1189 - DNA, 20 bp, Artificial Sequence
  Synthetic oligonucleotide
catcacgtagtcgcaacagt

SEQ ID NO: 1190 - DNA, 20 bp, Artificial Sequence
  Synthetic oligonucleotide
ttctcagtggaagcaaaata

SEQ ID NO: 1191 - DNA, 21 bp, Artificial Sequence
  Synthetic oligonucleotide
acatcactaggtttcaaactt

SEQ ID NO: 1192 - DNA, 17 bp, Artificial Sequence
  Synthetic oligonucleotide
ccagatgaccaaattgg

SEQ ID NO: 1193 - DNA, 18 bp, Artificial Sequence
  Synthetic oligonucleotide
atcacgcactaaattaat

SEQ ID NO: 1194 - DNA, 18 bp, Artificial Sequence
  Synthetic oligonucleotide
acttaccccaaaatgctg

SEQ ID NO: 1195 - DNA, 15 bp, Artificial Sequence
  Synthetic oligonucleotide
ttgccgaggcttctt

SEQ ID NO: 1196 - DNA, 18 bp, Artificial Sequence
  Synthetic oligonucleotide
caatttgcccccagcgct

SEQ ID NO: 1197 - DNA, 17 bp, Artificial Sequence
  Synthetic oligonucleotide
tctagcaggagaagttc

SEQ ID NO: 1198 - DNA, 22 bp, Artificial Sequence
  Synthetic oligonucleotide
actactttagattcgaagaccc

SEQ ID NO: 1199 - DNA, 19 bp, Artificial Sequence
  Synthetic oligonucleotide
aagtgtacgttgaaatcct

SEQ ID NO: 1200 - DNA, 20 bp, Artificial Sequence
  Synthetic oligonucleotide
cccagtccctacttattgtt

SEQ ID NO: 1201 - DNA, 19 bp, Artificial Sequence
  Synthetic oligonucleotide
aaatgctgttgttaaaatt

SEQ ID NO: 1202 - DNA, 21 bp, Artificial Sequence
  Synthetic oligonucleotide
ttgagttctggttgtaagatt

SEQ ID NO: 1203 - DNA, 16 bp, Artificial Sequence
  Synthetic oligonucleotide
tcgcgccccactgcgt

SEQ ID NO: 1204 - DNA, 18 bp, Artificial Sequence
  Synthetic oligonucleotide
ttggccgcaaattgcaca

SEQ ID NO: 1205 - DNA, 18 bp, Artificial Sequence
  Synthetic oligonucleotide
tcgtggtggtgacggtaa

SEQ ID NO: 1206 - DNA, 18 bp, Artificial Sequence
  Synthetic oligonucleotide
accaatggttctaaagcc

SEQ ID NO: 1207 - DNA, 18 bp, Artificial Sequence
  Synthetic oligonucleotide
ttgcggccaatgtttgta

SEQ ID NO: 1208 - DNA, 19 bp, Artificial Sequence
  Synthetic oligonucleotide
tacctattggcaaatctac

SEQ ID NO: 1209 - DNA, 20 bp, Artificial Sequence
  Synthetic oligonucleotide
caagtcctgagttgaatgta

SEQ ID NO: 1210 - DNA, 18 bp, Artificial Sequence
  Synthetic oligonucleotide
ctgggtcttcgaatctaa

SEQ ID NO: 1211 - DNA, 17 bp, Artificial Sequence
  Synthetic oligonucleotide
cattgccaaaaggcttc

SEQ ID NO: 1212 - DNA, 18 bp, Artificial Sequence
  Synthetic oligonucleotide
ttcggcaagactatgctc

SEQ ID NO: 1213 - DNA, 19 bp, Artificial Sequence
  Synthetic oligonucleotide
cacaagtagtggcaccttc

SEQ ID NO: 1214 - DNA, 20 bp, Artificial Sequence
  Synthetic oligonucleotide
aatggatcattacaaaattg

SEQ ID NO: 1215 - DNA, 17 bp, Artificial Sequence
  Synthetic oligonucleotide
caaacccaaggaaattt

SEQ ID NO: 1216 - DNA, 20 bp, Artificial Sequence
  Synthetic oligonucleotide
ttgtttttgtggtaataaac

SEQ ID NO: 1217 - DNA, 17 bp, Artificial Sequence
  Synthetic oligonucleotide
cctccaaaggcaatagt

SEQ ID NO: 1218 - DNA, 16 bp, Artificial Sequence
  Synthetic oligonucleotide
ctcgagggaatttaag

SEQ ID NO: 1219 - DNA, 21 bp, Artificial Sequence
  Synthetic oligonucleotide
ctagagactagtggcaataaa

SEQ ID NO: 1220 - DNA, 20 bp, Artificial Sequence
  Synthetic oligonucleotide
atcgtgctacaacttcctca

SEQ ID NO: 1221 - DNA, 18 bp, Artificial Sequence
  Synthetic oligonucleotide
ccgctctcactcaacatg

SEQ ID NO: 1222 - DNA, 20 bp, Artificial Sequence
  Synthetic oligonucleotide
atggtaggacagggttatca

SEQ ID NO: 1223 - DNA, 18 bp, Artificial Sequence
  Synthetic oligonucleotide
ccctatggtgctaacaaa

SEQ ID NO: 1224 - DNA, 18 bp, Artificial Sequence
  Synthetic oligonucleotide
attggatgacaaagatcc

SEQ ID NO: 1225 - DNA, 19 bp, Artificial Sequence
  Synthetic oligonucleotide
aactgaggatctgaaaact

SEQ ID NO: 1226 - DNA, 20 bp, Artificial Sequence
  Synthetic oligonucleotide
ctgggtcttcgaatctaaag

SEQ ID NO: 1227 - DNA, 18 bp, Artificial Sequence
  Synthetic oligonucleotide
cccctgcatacactaatt

SEQ ID NO: 1228 - DNA, 18 bp, Artificial Sequence
  Synthetic oligonucleotide
cctaggttgaagataacc

SEQ ID NO: 1229 - DNA, 17 bp, Artificial Sequence
  Synthetic oligonucleotide
ctctgctcccttctgcg

SEQ ID NO: 1230 - DNA, 21 bp, Artificial Sequence
  Synthetic oligonucleotide
cctcttctcgttcctcatcac

SEQ ID NO: 1231 - DNA, 21 bp, Artificial Sequence
  Synthetic oligonucleotide
cctgtatggttacaacctatg

SEQ ID NO: 1232 - DNA, 21 bp, Artificial Sequence
  Synthetic oligonucleotide
caaaagatcacattggcaccc

SEQ ID NO: 1233 - DNA, 19 bp, Artificial Sequence
  Synthetic oligonucleotide
acgcaactggatagacaga

SEQ ID NO: 1234 - DNA, 20 bp, Artificial Sequence
  Synthetic oligonucleotide
cttccttgccatgttgagtg

SEQ ID NO: 1235 - DNA, 17 bp, Artificial Sequence
  Synthetic oligonucleotide
ctgctcatggattgttg

SEQ ID NO: 1236 - DNA, 21 bp, Artificial Sequence
  Synthetic oligonucleotide
aaggctgagagacatattcaa

SEQ ID NO: 1237 - DNA, 25 bp, Artificial Sequence
  Synthetic oligonucleotide
cctaggacttttctattaaaatata

SEQ ID NO: 1238 - DNA, 19 bp, Artificial Sequence
  Synthetic oligonucleotide
cattggcatggaagtcaca

SEQ ID NO: 1239 - DNA, 20 bp, Artificial Sequence
  Synthetic oligonucleotide
taccgtcaccaccacgaatt

SEQ ID NO: 1240 - DNA, 21 bp, Artificial Sequence
  Synthetic oligonucleotide
cagtccaagatggtatttcta

SEQ ID NO: 1241 - DNA, 22 bp, Artificial Sequence
  Synthetic oligonucleotide
aaaatggatcattacaaaattg

SEQ ID NO: 1242 - DNA, 20 bp, Artificial Sequence
  Synthetic oligonucleotide
atgctggacaataaatttta

SEQ ID NO: 1243 - DNA, 16 bp, Artificial Sequence
  Synthetic oligonucleotide
ttggcaaagaaatttg

SEQ ID NO: 1244 - DNA, 18 bp, Artificial Sequence
  Synthetic oligonucleotide
acgtggtgtttattaccc

SEQ ID NO: 1245 - DNA, 18 bp, Artificial Sequence
  Synthetic oligonucleotide
ttgatcgcgccccactgc

SEQ ID NO: 1246 - DNA, 20 bp, Artificial Sequence
  Synthetic oligonucleotide
tcgcactagaataaactctg

SEQ ID NO: 1247 - DNA, 19 bp, Artificial Sequence
  Synthetic oligonucleotide
cagtccagatgaccaaatt

SEQ ID NO: 1248 - DNA, 17 bp, Artificial Sequence
  Synthetic oligonucleotide
ctcctggtgattcttct

SEQ ID NO: 1249 - DNA, 17 bp, Artificial Sequence
  Synthetic oligonucleotide
tcggccccaaggtttac

SEQ ID NO: 1250 - DNA, 19 bp, Artificial Sequence
  Synthetic oligonucleotide
aagggaaatacaaaatttg

SEQ ID NO: 1251 - DNA, 18 bp, Artificial Sequence
  Synthetic oligonucleotide
caggtgccatcaaattgg

SEQ ID NO: 1252 - DNA, 17 bp, Artificial Sequence
  Synthetic oligonucleotide
ctgtgcacttgaccctc

SEQ ID NO: 1253 - DNA, 18 bp, Artificial Sequence
  Synthetic oligonucleotide
taggtcaaccacgttccc

SEQ ID NO: 1254 - DNA, 21 bp, Artificial Sequence
  Synthetic oligonucleotide
cataataagaggctggatttt

SEQ ID NO: 1255 - DNA, 17 bp, Artificial Sequence
  Synthetic oligonucleotide
ttgactgccgcctctgc

SEQ ID NO: 1256 - DNA, 19 bp, Artificial Sequence
  Synthetic oligonucleotide
acaatttgcccccagcgct

SEQ ID NO: 1257 - DNA, 16 bp, Artificial Sequence
  Synthetic oligonucleotide
aaacccaaggaaattt

SEQ ID NO: 1258 - DNA, 19 bp, Artificial Sequence
  Synthetic oligonucleotide
aacgtactgccactaaagc

SEQ ID NO: 1259 - DNA, 22 bp, Artificial Sequence
  Synthetic oligonucleotide
ctagtcagtgtgttaatcttac

SEQ ID NO: 1260 - DNA, 20 bp, Artificial Sequence
  Synthetic oligonucleotide
ccaacaacacctgtatggtt

SEQ ID NO: 1261 - DNA, 19 bp, Artificial Sequence
  Synthetic oligonucleotide
aaggctccctcagttgcaa

SEQ ID NO: 1262 - DNA, 20 bp, Artificial Sequence
  Synthetic oligonucleotide
tacaaccagaactcaattac

SEQ ID NO: 1263 - DNA, 20 bp, Artificial Sequence
  Synthetic oligonucleotide
ttgggttccacgtgctagcg

SEQ ID NO: 1264 - DNA, 21 bp, Artificial Sequence
  Synthetic oligonucleotide
ctgtaatggttccattttcat

SEQ ID NO: 1265 - DNA, 20 bp, Artificial Sequence
  Synthetic oligonucleotide
ttgcggccaatgtttgtaat

SEQ ID NO: 1266 - DNA, 20 bp, Artificial Sequence
  Synthetic oligonucleotide
aatgaatctggcttgaaaac

SEQ ID NO: 1267 - DNA, 17 bp, Artificial Sequence
  Synthetic oligonucleotide
atgcgcgacattccgaa

SEQ ID NO: 1268 - DNA, 16 bp, Artificial Sequence
  Synthetic oligonucleotide
cctggagttgaatttc

SEQ ID NO: 1269 - DNA, 18 bp, Artificial Sequence
  Synthetic oligonucleotide
ttgcgtcaccaaatgaat

SEQ ID NO: 1270 - DNA, 19 bp, Artificial Sequence
  Synthetic oligonucleotide
acctattggcaaatctacc

SEQ ID NO: 1271 - DNA, 21 bp, Artificial Sequence
  Synthetic oligonucleotide
atgtaaagcaagtaaagtttg

SEQ ID NO: 1272 - DNA, 18 bp, Artificial Sequence
  Synthetic oligonucleotide
acgcatacaaaacattcc

SEQ ID NO: 1273 - DNA, 16 bp, Artificial Sequence
  Synthetic oligonucleotide
tcctgctgcagatttg

SEQ ID NO: 1274 - DNA, 19 bp, Artificial Sequence
  Synthetic oligonucleotide
atagtcttgccgaatacca

SEQ ID NO: 1275 - DNA, 19 bp, Artificial Sequence
  Synthetic oligonucleotide
cagacgggcgattttgtta

SEQ ID NO: 1276 - DNA, 24 bp, Artificial Sequence
  Synthetic oligonucleotide
cttgctttacatagaagttatttg

SEQ ID NO: 1277 - DNA, 17 bp, Artificial Sequence
  Synthetic oligonucleotide
cagtggaagcaaaataa

SEQ ID NO: 1278 - DNA, 20 bp, Artificial Sequence
  Synthetic oligonucleotide
aaaggcaatagtgcgaccac

SEQ ID NO: 1279 - DNA, 23 bp, Artificial Sequence
  Synthetic oligonucleotide
ctgtgactcttcttcctgctgca

SEQ ID NO: 1280 - DNA, 19 bp, Artificial Sequence
  Synthetic oligonucleotide
caagcctcttctcgttcct

SEQ ID NO: 1281 - DNA, 20 bp, Artificial Sequence
  Synthetic oligonucleotide
ttggtgacgcaactggatag

SEQ ID NO: 1282 - DNA, 21 bp, Artificial Sequence
  Synthetic oligonucleotide
acacactgactagagactagt

SEQ ID NO: 1283 - DNA, 20 bp, Artificial Sequence
  Synthetic oligonucleotide
caattaccccctgcatacac

SEQ ID NO: 1284 - DNA, 19 bp, Artificial Sequence
  Synthetic oligonucleotide
ttgcggccaatgtttgtaa

SEQ ID NO: 1285 - DNA, 19 bp, Artificial Sequence
  Synthetic oligonucleotide
ttactgccagttgaatctg

SEQ ID NO: 1286 - DNA, 20 bp, Artificial Sequence
  Synthetic oligonucleotide
aaccctgtcctaccatttaa

SEQ ID NO: 1287 - DNA, 21 bp, Artificial Sequence
  Synthetic oligonucleotide
tattcttaaacacaaattccc

SEQ ID NO: 1288 - DNA, 20 bp, Artificial Sequence
  Synthetic oligonucleotide
cttgaccctctctcagaaac

SEQ ID NO: 1289 - DNA, 18 bp, Artificial Sequence
  Synthetic oligonucleotide
cattcccaccaacagagc

SEQ ID NO: 1290 - DNA, 19 bp, Artificial Sequence
  Synthetic oligonucleotide
ctggcagtaaccagaatgg

SEQ ID NO: 1291 - DNA, 14 bp, Artificial Sequence
  Synthetic oligonucleotide
acccgcaatcctgc

SEQ ID NO: 1292 - DNA, 16 bp, Artificial Sequence
  Synthetic oligonucleotide
ccgcctctgctccctt

SEQ ID NO: 1293 - DNA, 22 bp, Artificial Sequence
  Synthetic oligonucleotide
taggcgtgtgcttagaatatat

SEQ ID NO: 1294 - DNA, 15 bp, Artificial Sequence
  Synthetic oligonucleotide
ccacttgcgaatttt

SEQ ID NO: 1295 - DNA, 18 bp, Artificial Sequence
  Synthetic oligonucleotide
ctttccatccaacttttg

SEQ ID NO: 1296 - DNA, 29 bp, Artificial Sequence
  Synthetic oligonucleotide
ctgagctttcatggccttctgctatttca

SEQ ID NO: 1297 - DNA, 18 bp, Artificial Sequence
  Synthetic oligonucleotide
actgggcacggtgagcgt

SEQ ID NO: 1298 - DNA, 28 bp, Artificial Sequence
  Synthetic oligonucleotide
tctggagccacctctcccatttctttta

SEQ ID NO: 1299 - DNA, 27 bp, Artificial Sequence
  Synthetic oligonucleotide
acaagcttcacgaaggctccacataca

SEQ ID NO: 1300 - DNA, 24 bp, Artificial Sequence
  Synthetic oligonucleotide
atccattcagcgaccccgttagcg

SEQ ID NO: 1301-1310 - skipped (10 entries)

SEQ ID NO: 1311 - DNA, 20 bp, Artificial Sequence
  Synthetic oligonucleotide
gaccccaaaatcagcgaaat

SEQ ID NO: 1312 - DNA, 24 bp, Artificial Sequence
  Synthetic oligonucleotide
tctggttactgccagttgaatctg

SEQ ID NO: 1313 - DNA, 24 bp, Artificial Sequence
  Synthetic oligonucleotide
accccgcattacgtttggtggacc

SEQ ID NO: 1314 - DNA, 20 bp, Artificial Sequence
  Synthetic oligonucleotide
ttacaaacattggccgcaaa

SEQ ID NO: 1315 - DNA, 18 bp, Artificial Sequence
  Synthetic oligonucleotide
gcgcgacattccgaagaa

SEQ ID NO: 1316 - DNA, 23 bp, Artificial Sequence
  Synthetic oligonucleotide
acaatttgcccccagcgcttcag

SEQ ID NO: 1317 - DNA, 19 bp, Artificial Sequence
  Synthetic oligonucleotide
agatttggacctgcgagcg

SEQ ID NO: 1318 - DNA, 20 bp, Artificial Sequence
  Synthetic oligonucleotide
gagcggctgtctccacaagt

SEQ ID NO: 1319 - DNA, 23 bp, Artificial Sequence
  Synthetic oligonucleotide
ttctgacctgaaggctctgcgcg